SEQUENCE LISTING (2 sequences)

SEQ ID NO: 1 - DNA, 20 bp, Artificial Sequence
  Chemically synthesized
gttctgtgcgatacgtctcc

SEQ ID NO: 2 - DNA, 21 bp, Artificial Sequence
  Chemically synthesized
gcctttcacaggcagatattc

(19) United States
(12) Patent Application Publication
(10) Pub. No.: US 2020/0113950 A1
(43) Pub. Date: Apr. 16, 2020

(54) Human microbiota derived N-acyl amides for the treatment of human disease

(71) Applicant: The Rockefeller University, New York, NY (US)

(72) Inventors: Louis Cohen, New York, NY (US); Sean Brady, New York, NY (US)

(21) Appl. No.: 16/627,440

(22) PCT Filed: Jun. 29, 2018
(86) PCT No.: PCT/US18/40195

§ 371 Date: Dec. 30, 2019

Related U.S. Application Data
(60) Provisional application No. 62/527,314, filed on Jun. 30, 2017.

(51) Int. Cl. A61K 35/741; A61K 31/16; A61K 38/45; C12N 1/20; C12N 15/70

(52) U.S. Cl. CPC A61K 35/741; A61K 31/16; C12N 15/70; C12N 1/20; A61K 38/45

(57) ABSTRACT

The present invention provides compositions and methods for the modulation of G protein-coupled receptors (GPCRs).

### CROSS-REFERENCE TO RELATED APPLICATIONS

This application claims priority to and the benefit of U.S. Application No. 62/527,314, filed Jun. 30, 2017, the content of which is incorporated in its entirety herein.

### STATEMENT REGARDING FEDERALLY SPONSORED RESEARCH OR DEVELOPMENT

This invention was made with government support under grant no. DK109287 awarded by the National Institutes of Health. The Government has certain rights in the invention.

### BACKGROUND OF THE INVENTION

The human microbiome is believed to play an important role in both normal physiology and disease. Despite direct evidence linking resident bacteria to disease pathophysiology in mice and correlative evidence in humans, the mechanisms by which bacteria affect mammalian physiology remain poorly defined (Koppel et al., 2016, Cell Chem Biol 23:18-30). Bacteria rely heavily on small molecules (natural products) to interact with their environment (Meinwald et al., 2008, PNAS 105:5439-40). While it is likely that the human microbiota similarly relies on small molecules to interact with its human host, the identity and functions of microbiota-encoded effector molecules are largely unknown. The study of small molecules produced by human microbiota and the identification of host receptors they interact with should help to define the relationship between bacteria and human physiology and provide a novel resource for the discovery of small molecule therapeutics.

Commensal bacteria are believed to play important roles in human health; however, the mechanisms by which they affect mammalian physiology are poorly understood. Bacterial metabolites are likely to be key components of host microbiota interactions.

The discovery of commendamide, a human microbiota encoded, G protein-coupled receptor (GPCR) active, long-chain N-acyl amide that suggests a structural convergence between human signaling molecules and metabolites produced by commensal bacteria (Cohen et al., 2015, PNAS 112:E4825-34). Long-chain N-acyl amides, like the endocannabinoids, are an important class of human signaling molecules that help to control immunity, behavior and metabolism, among other aspects of human physiology (Hanuš et al., 2014, BioFactors 40:381-8). N-acyl amides are able to regulate such diverse human cellular functions due, in part, to their ability to interact with GPCRs. GPCRs are the largest family of membrane receptors in eukaryotes and are likely to be key mediators of host-microbial interactions in the human microbiome. The importance of GPCRs to human physiology is reflected by the fact that they are the most common targets of therapeutically approved small molecule drugs and that the GPCRs with which human N-acyl amides interact are involved in diseases including diabetes, obesity, cancer, and inflammatory bowel disease among others (Cani et al., 2015, Nat Rev Endocrinol 12:133-43; Pacher et al., 2013, FEBS J 280:1918-43). With numerous possible combinations of amine head groups and acyl tails, long-chain N-acyl amides represent a potentially large and functionally diverse class of microbiota-encoded GPCR-active signaling molecules.

Thus, there is thus a need in the art for compositions and methods that modulate GPCRs. The present invention addresses this unmet need in the art.

### SUMMARY OF THE INVENTION

In one aspect, the invention provides a genetically engineered cell, wherein the cell expresses a human microbial N-acyl synthase (hm-NAS) gene. In one embodiment, the cell is a non-pathogenic bacterial cell. In one embodiment, the cell is capable of producing a N-acyl amide. In one embodiment, the hm-NAS gene is selected from a hm-NAS gene of table 1 or table 2. In one embodiment, the hm-NAS gene is N-acyl serinol synthase.

In one embodiment, the invention provides a probiotic composition. In one embodiment, the probiotic composition comprises a genetically engineered cell of the invention. In one embodiment, the composition further comprises a prebiotic.

In one aspect, the invention provides a method for modulating a G protein-coupled receptor (GPCR) activity in a subject. In one embodiment, the method comprises administering to the subject an effective amount of a composition comprising at least one selected from the group consisting of a genetically engineered cell, an hm-NAS gene, and a N-acyl amide. In one embodiment, the engineered cell expresses a human microbial N-acyl synthase (hm-NAS) gene.

In one embodiment, the hm-NAS gene is selected from a hm-NAS gene of table 1 or table 2. In one embodiment, the hm-NAS gene is N-acyl serinol synthase.

In one embodiment, the N-acyl amide is represented by Formula (1):

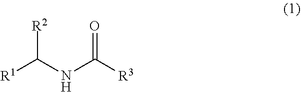

wherein R¹ is selected from the group consisting of carboxylate and CH₂OH;

R² is selected from the group consisting of H, (C₃-C₄)alkyl-NH₃⁺, (C₃-C₄)alkyl-NH₂, C₂ alkyl-C(═O)NH₂, CH₂OH, and methyl; and

R³ is selected from the group consisting of (C₉-C₁₈)alkyl, (C₉-C₁₈)alkenyl, wherein the (C₉-C₁₈)alkyl and (C₉-C₁₈)alkenyl are optionally substituted.

In one embodiment, the GPCR is enriched in the gastrointestinal mucosa. In one embodiment, the GPCR is selected from the group consisting of ADCYAP1R1, ADORA3, ADRA1B, ADRA2A, ADRA2B, ADRA2C, ADRB1, ADRB2, AGTR1, AGTRL1, AVPR1A, AVPR1B, AVPR2, BAI1, BAI2, BAI3, BDKRB1, BDKRB2, BRS3, C3AR1, C5AR1, C5L2, CALCR, CALCRL-RAMP1, CALCRL-RAMP2, CALCRL-RAMP3, CALCR-RAMP2, CALCR-RAMP3, CCKAR, CCKBR, CCR1, CCR10, CCR2, CCR3, CCR4, CCR5, CCR6, CCR7, CCR8, CCR9, CCRL2, CHRM1, CHRM2, CHRM3, CHRM4, CHRM5, CMKLR1, CNR1, CNR2, CRHR1, CRHR2, CRTH2, CX3CR1, CXCR1, CXCR2, CXCR3, CXCR4, CXCR5, CXCR6, CXCR7, DARC, DRD1, DRD2L, DRD2S, DRD3, DRD4, DRD5, EBI2, EDG1, EDG3, EDG4, EDG5, EDG6, EDG7, EDNRA, EDNRB, F2R, F2RL1, F2RL3, FFAR1, FPR1, FPRL1, FSHR, G2A, GALR1, GALR2, GCGR, GHSR, GHSR1B, GIPR, GLP1R, GLP2R, GPR1, GPR101, GPR103, GPR107, GPR109A, GPR109B, GPR119, GPR12, GPR120, GPR123, GPR132, GPR135, GPR137, GPR139, GPR141, GPR142, GPR143, GPR146, GPR148, GPR149, GPR15, GPR150, GPR151, GPR152, GPR157, GPR161, GPR162, GPR17, GPR171, GPR173, GPR176, GPR18, GPR182, GPR20, GPR23, GPR25, GPR26, GPR27, GPR3, GPR30, GPR31, GPR32, GPR35, GPR37, GPR37L1, GPR39, GPR4, GPR45, GPR50, GPR52, GPR55, GPR6, GPR61, GPR65, GPR75, GPR78, GPR79, GPR83, GPR84, GPR85, GPR88, GPR91, GPR92, GPR97, GRPR, HCRTR1, HCRTR2, HRH1, HRH2, HRH3, HRH4, HTR1A, HTR1B, HTR1E, HTR1F, HTR2A, HTR2C, HTR5A, KISS1R, LGR4, LGR5, LGR6, LHCGR, LTB4R, MC1R, MC3R, MC4R, MC5R, MCHR1, MCHR2, MLNR, MRGPRD, MRGPRE, MRGPRF, MRGPRX1, MRGPRX2, MRGPRX4, MTNR1A, NMBR, NMU1R, NPBWR1, NPBWR2, NPFFR1, NPSR1B, NPY1R, NPY2R, NTSR1, OPN5, OPRD1, OPRK1, OPRL1, OPRM1, OXER1, OXGR1, OXTR, P2RY1, P2RY11, P2RY12, P2RY2, P2RY4, P2RY6, P2RY8, PPYR1, PRLHR, PROKR1, PROKR2, PTAFR, PTGER2, PTGER3, PTGER4, PTGFR, PTGIR, PTHR1, PTHR2, RXFP3, SCTR, SPR4, SSTR1, SSTR2, SSTR3, SSTR5, TAAR5, TACR1, TACR2, TACR3, TBXA2R, TRHR, TSHR(L), UTR2, VIPR1, and VIPR2. In one embodiment, the GPCR is selected from the group consisting of GPR119, SPR4, G2A, PTGIR, and PTGER4.

In one embodiment, the GPCR activity is reduced. In one embodiment, the GPCR activity is increased.

In one aspect, the invention provides a method for treating a disease or disorder in a subject. In one embodiment, the method comprises administering to a subject a therapeutically effective amount of a composition comprising at least one selected from the group consisting of a genetically engineered cell, an hm-NAS gene, and a N-acyl amide. In one embodiment, the cell expresses a human microbial N-acyl synthase (hm-NAS) gene.

In one embodiment, the hm-NAS gene is selected from a hm-NAS gene of table 1 or table 2. In one embodiment, the hm-NAS gene is N-acyl serinol synthase.

In one embodiment, the N-acyl amide is represented by Formula (1):

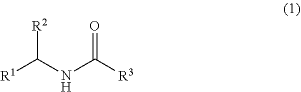

wherein R¹ is selected from the group consisting of carboxylate and CH₂OH;

R² is selected from the group consisting of H, (C₃-C₄)alkyl-NH₃⁺, (C₃-C₄)alkyl-NH₂, C2 alkyl-C(═O)NH₂, CH₂OH, and methyl; and

R³ is selected from the group consisting of (C₉-C₁₈)alkyl, (C₉-C₁₈)alkenyl, wherein the (C₉-C₁₈)alkyl and (C₉-C₁₈)alkenyl are optionally substituted.

In one embodiment, the disease or disorder is selected from the group consisting of diabetes, obesity, colitis, autoimmune disorder, atherosclerosis, gastrophoresis, cirrhosis, non-alcoholic fatty liver disease, non-alcoholic steatohepatitis, and osteopenia. In one embodiment, the disease or disorder is associated with abnormal gastric emptying, appetite, or glucose homeostasis.

In one embodiment, the subject is a mammal. In one embodiment, the subject is a human.

In one aspect, the invention provides a gene therapy vector. In one embodiment, the gene therapy vector comprises a nucleic acid expression cassette, wherein the nucleic acid expression cassette comprises a sequence of a hm-NAS gene or a sequence having at least 90% homology to a hm-NAS gene.

In one embodiment, the hm-NAS gene is selected from a hm-NAS gene of table 1 or table 2.

In one embodiment, the gene therapy vector is selected from the group consisting of a lentiviral vector, a retroviral vector and an adenoviral vector.

In one aspect, the invention provides a composition comprising an N-acyl amide. In one embodiment, the N-acyl amide is represented by Formula (1):

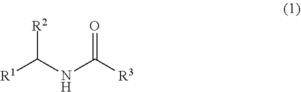

wherein R¹ is selected from the group consisting of carboxylate and CH₂OH;

R² is selected from the group consisting of H, (C₃-C₄)alkyl-NH₃⁺, (C₃-C₄)alkyl-NH₂, C2 alkyl-C(═O)NH₂, CH₂OH, and methyl; and

R³ is selected from the group consisting of (C₉-C₁₈)alkyl, (C₉-C₁₈)alkenyl, wherein the (C₉-C₁₈)alkyl and (C₉-C₁₈)alkenyl are optionally substituted.

In one embodiment, Formula (1) is represented by one of Formulae (2)-(6):

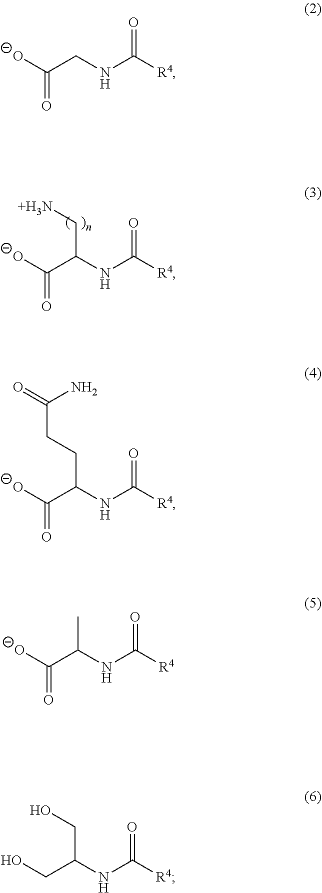

wherein R⁴ is selected from the group consisting of (C₉-C₁₈)alkyl, (C₉-C₁₈)alkenyl, wherein the (C₉-C₁₈)alkyl and (C₉-C₁₈)alkenyl are optionally substituted; and

n is 3 or 4.

In one embodiment, Formulae (2)-(6) are represented by Formulae (7)-(11):

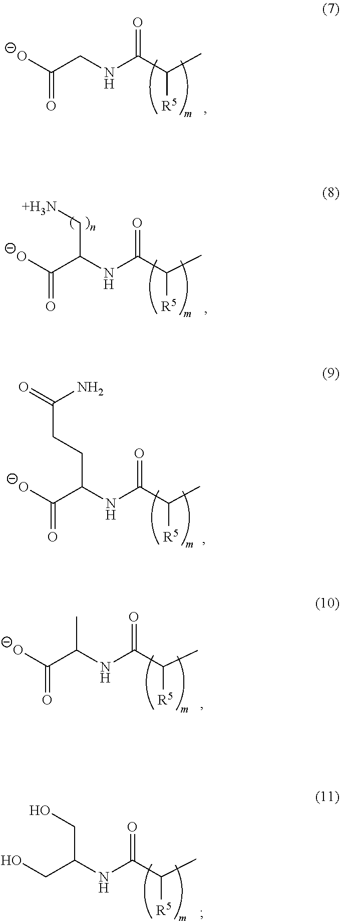

wherein each occurrence of R⁵ is independently selected from the group consisting of H and —OH;

and m is an integer from 8 to 17.

In one embodiment, Formulae (2)-(6) are represented by Formulae (12)-(16)

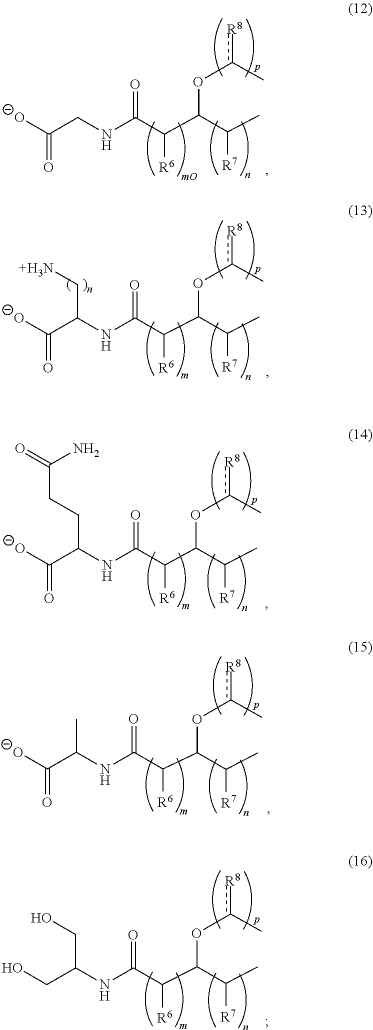

wherein each occurrence of R⁶, R⁷, and R⁸ is independently selected from the group consisting of H, —OH, and (═O);

m is an integer from 1 to 5;

n is an integer from 2 to 15;

p is an integer from 8 to 18; and

q is an integer from 3 to 4.

In one embodiment, the N-acyl amide is selected from the group consisting of:

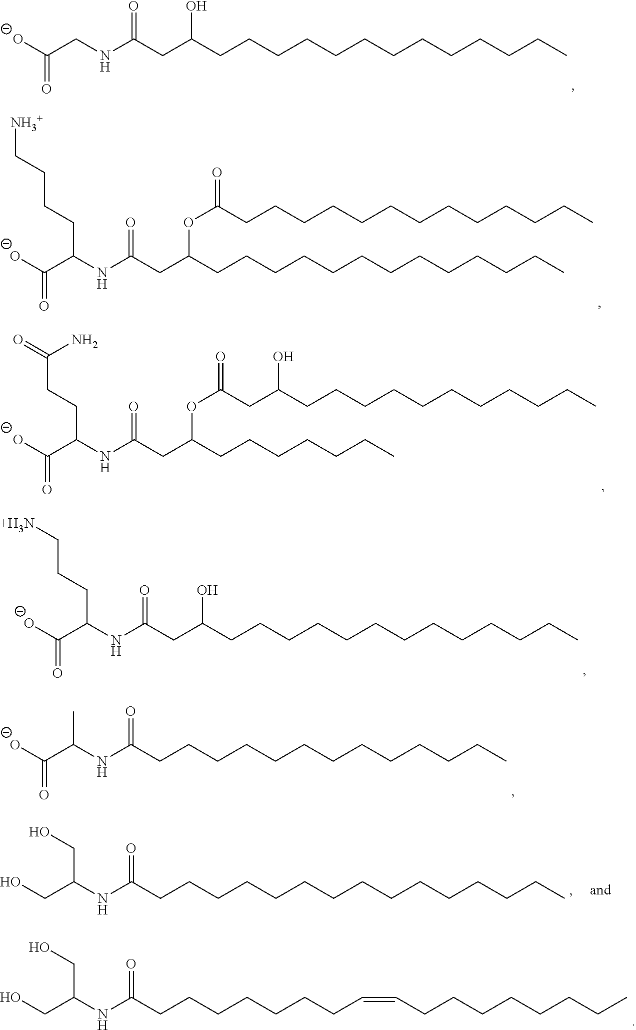

In one embodiment, the composition further comprises a pharmaceutically acceptable carrier. In one embodiment, the composition is formulated as a probiotic.

### BRIEF DESCRIPTION OF THE DRAWINGS

The following detailed description of embodiments of the invention will be better understood when read in conjunction with the appended drawings. It should be understood, however, that the invention is not limited to the precise arrangements and instrumentalities of the embodiments shown in the drawings.

FIG. 1, comprising FIG. 1A through FIG. 1C, depicts experimental results demonstrating that N-acyls are enriched in gut microbiota. FIG. 1A depicts a phylogenetic tree of N-acyl transferase genes from PFAM13444. hm-NAS genes are identified by a colored circle at the tip of the branch. Black dots were not synthesized, red dots were synthesized but no molecule was produced in the heterologous expression experiment and large grey dots mark genes that produced N-acyl amides in a heterologous expression experiment. Branches are colored by phylogenetic distribution. FIG. 1B depicts the major metabolite from each of the 6 N-acyl families (1-6) identified in heterologous expression experiments. FIG. 1C depicts hm-NAS gene distribution and abundance (Reads per Kilobase of Gene Per Million Reads (RPKM)) in the human microbiome based on the encoded molecule family (1-6).

FIG. 2, comprising FIG. 2A and FIG. 2B, depicts experimental results demonstrating N-acyl synthase gene expression in vivo. FIG. 2A depicts gene expression analysis for an N-acyl glycine hm-NAS gene in a stool metatranscriptome dataset and an N-acyloxyacyl ornithine/lysine hm-NAS gene in a supragingival plaque metatranscriptome dataset. Gene expression is normalized to the expression of all genes from a bacterial genome containing the hm-NAS gene that was heterologously expressed —Bacteroides dorei in stool, Capnocytophaga ochracea in plaque (1 highly expressed, 0 not expressed). FIG. 2B depicts a comparison of hm-NAS gene abundance based on RNA or DNA derived reads obtained from individual patient stool samples. Abundance is measured in RPKM.

FIG. 3, comprising FIG. 3A through FIG. 3C, depicts experimental results demonstrating that hm-N-acyls mimic endogenous GPCR ligands. FIG. 3A depicts a screen of N-acyl amides for agonist activity against 168 GPCRs with known ligands. FIG. 3B depicts a screen of N-acyl amides for agonist activity against 72 orphan GPCRs. The dot plots display all data for all N-acyl amides against all GPCRs. Data for each N-acyl amide is displayed in a different color. Bar graphs show the strongest N-acyl GPCR agonist interactions compared to all GPCRs. N-acyl GPCR interactions are specific to that receptor. Inset to the bar graphs are dose response curves and EC50 data for N-acyl amides against specific GPCRs. The S1PR4 bar graph is for N-3-hydroxypalmitoyl lysine, which like N-3-hydroxypalmitoyl ornithine is encoded by the same hm-NAS gene and is a specific agonist of S1PR4. Inset dose response curve is for N-3-hydroxypalmiotyl ornithine. FIG. 3C depicts a screen of N-acyl amides as antagonists against 168 GPCRs in the presence of their endogenous ligands. The most potent observed antagonist activity (red bars/dots) was N-acyloxyacyl glutamine inhibition of two prostaglandin receptors. PTGIR is specifically inhibited by N-acyloxyacyl glutamine. PTGER4 is inhibited by structurally diverse hm-N-acyl amides

FIG. 4 depicts the structural mimicry of GPCR ligands. Comparison of microbiota encoded and human GPCR ligands suggests a structural and functional complementarity.

FIG. 5, comprising FIG. 5A through FIG. 5H, depicts experimental results demonstrating N-acyl serinols affect GLP-1 secretion in vitro and glucose homeostasis in vivo. FIG. 5A depicts β-arrestin GPR119 activation assay using microbiota (green) and human (blue) ligands (each dose performed in duplicate). FIG. 5B depicts β-arrestin assay comparing microbiota ligands and 20 synthesized N-palmitoyl amino acids (screen performed in singlicate). FIG. 5C depicts release of GLP-1 by GLUTag cells (ANOVA, p<0.05, data combined from 2 independent experiments, N=4 for DMSO and 2-oleoyl glycerol, N=6 for OEA and N-oleoyl serinol). FIG. 5D depicts oral glucose tolerance test (OGTT) in gnotobiotic mice. Treatment mice (n=6 mice, data combined from 2 independent experiments) were colonized with E. coli producing N-acyl serinols and control mice (n=8 mice, data combined from 2 independent experiments) were colonized with E. coli containing an empty vector (two-way ANOVA, bonferroni post-hoc) FIG. 5E depicts OGTT after withholding IPTG to stop N-acyl gene expression (no difference, two-way ANOVA, N is the same as in FIG. 5D). FIG. 5F depicts OGTT in an antibiotic treated mouse cohort (n=9 mice in both groups, data combined from 2 independent experiments, two-way ANOVA, bonferroni post-hoc). FIG. 5G depicts insulin (n=6 mice in both groups, one experiment, technical triplicates) measured at 15 min after glucose gavage in the antibiotic treated cohort (unpaired T test, two tailed). FIG. 5H depicts GLP-1 (n=9 control mice, n=10 treatment mice, data combined from 2 independent experiments, technical replicates) measured at 15 min after glucose gavage in the antibiotic treated cohort (unpaired T test, two tailed). Error bars (mean+/−SEM)*p<0.05, **p<0.01***p<0.001.

FIG. 6, comprising FIG. 6A through FIG. 6C, depicts an analysis of hm-NAS clone families. FIG. 6A depicts LCMS analysis of crude extracts prepared from E. coli transformed with each hm-NAS gene expression construct (number 1-43, see Table 3 for details about each clone number) compared to negative control extracts derived from E. coli containing an empty vector (con). Based on metabolite retention time and observed mass hm-NAS genes could be grouped into 6 N-acyl amide families (1-6). The mass of the major metabolite (pictured) from each N-acyl amide family is shown in either the ESI(+) or ESI(−) MS detection mode for each hm-NAS extract including the control extract. Functional differences in NAS enzymes follow the pattern of the NAS phylogenetic tree, with hm-NAS genes from the same clade or sub-clade largely encoding the same metabolite family. Commendamide was previously isolated and is part of family 1. FIG. 6B depicts a phylogenetic tree of PFAM13444 showing the location of each hm-NAS gene that was synthesized and examined by heterologous expression. FIG. 6C depicts crude ethyl acetate extracts were prepared from cultures of bacterial species that harbor the same or highly related (>80% nucleotide identity) hm-NAS gene that was expressed by heterologous expression. The only exception was for N-acyl alanines for which a representative cultured commensal bacterial species was not available. N-acyl glycines were previously analyzed in the same manner. The extracted ion for the hm-NAS gene family is shown for the E. coli clone compared to the crude extract from the commensal species.

FIG. 7 depicts the proposed two-step biosynthesis of N-acyl serinol using the two domains found in the enzyme predicted to be encoded by the hm-NAS N-acyl serinol synthase gene. Simple N-palmitoyl derivatives of all 20 natural amino acids did not activated GPR119 by more than 37% relative to OEA.

FIG. 8, comprising FIG. 8A through FIG. 8E, depicts the validation of hits from the high throughput GPCR screen. When structural analogs were independently screened in the GPCR panel (e.g., N-oleoyl and palmitoyl serinol or N-3-hydroxypalmitoyl lysine and ornithine) they yielded the same GPCR profile and when N-acyl serinol was re-assayed across all GPCRs in the panel, it also yielded the same GPCR activity profile. FIG. 8A depicts results demonstrating that N-3-hydroxypalmitoyl lysine interacts with S1PR4. FIG. 8B depicts results demonstrating that N-3-hydroxypalmitoyl ornithine interacts with S1PR4. FIG. 8C depicts results demonstrating that N-palmitoyl serinol interacts with GPR119. FIG. 8D depicts results demonstrating that N-palmitoyl serinol interacts with GPR119 FIG. 8E depicts results demonstrating that N-palmitoyl oleoyl interacts with GPR119. Screening data performed in singlicate, dose response curves performed in duplicate. Error bars are mean+/−SEM.

FIG. 9 depicts a combined analysis of protein and transcript expression of GPCR in the gastrointestinal tract. Table links GPCR, N-acyl amide, bacterial genus and the site where these co-occur in the gastrointestinal tract (colored). Based on protein expression data (Human Protein Atlas) GPR119 is most highly expressed in the pancreas and duodenum, S1PR4 in the spleen and lymph node, G2A in the lymph node and appendix, PTGIR in the lung and appendix and PTGER4 in the bone marrow and small intestine. From gene expression data in the colon (GTEx dataset, N=88 patient samples from small intestine, 345 patient samples from colon) GPR132, PTGER4, and PTGIR are all expressed alongside the N-acyl synthase genes known to encode metabolites that target these GPCR (FIG. 1). In the gastrointestinal tract GPR119 and S1PR4 are most highly expressed in the small intestine where 16S studies have identified bacteria from the genera Gemella and Neisseria. All known reference genomes (NCBI) from these genera contain N-acyl synthase genes that are highly similar (blastN, e value 2e-132) to those we found to encode GPR119 or S1PR4 ligands.

FIG. 10 depicts a secondary assay of GPR119. ACTOne HEK293 cells (control) and ACTOne HEK293 cells transfected with GPR119 were exposed to equimolar concentrations of the endogenous GPR119 ligand oleoylethanolamide or the bacterial ligand N-oleoyl serinol. Relative fluorescent intensity was recorded for each ligand concentration compared to background signal. All data points were performed in quadruplicate and error bars represent SD around the mean. An increase in cAMP concentration was observed in HEK293 cells expressing GPR119 but not in native HEK293 cells. The DCEA [5-(N-Ethylcarboxamido)adenosine] control is presented to confirm cAMP response of the parental cell line. The EC50 for N-oleoyl serinol (bacterial) was 1.6 μM and for oleoylethanolamide was 5.1 μM, which are consistent with data from the β-arrestin assay (FIG. 5A).

FIG. 11, comprising FIG. 11A and FIG. 11B, depicts the identification of N-acyl serinol biosynthesis in vivo. FIG. 11A depicts LC-MS analysis of crude cecal extracts. Extracted-ion chromatograms for palmitoyl serinol ([M+H]⁺m/z: 330.3003) are shown. A peak with the same exact mass and chromatographic retention time as the N-palmitoyl serinol standard was present in treatment mice but not control mice. Treatment mice were colonized with E. coli containing the N-acyl serinol synthase gene. Control mice were colonized with E. coli containing the empty pET28c vector. FIG. 11B depicts identification of N-palmitoyl serinol by MS/MS fragmentation of the m/z 330.3003 ion. In the MS2 spectrum the diamond indicates N-palmitoyl serinol parent ion and the product ion at m/z: 92.0706 shows presence of the serinol head group.

FIG. 12, comprising FIG. 12A and FIG. 12B, depicts bacterial colonization of mouse model systems. One week after inoculation with E. coli a single fecal pellet from a colonized mouse was collected, resuspended in 400 μL PBS and plated at a 1/100 dilution onto LB agar plates with or without kanamycin 50 μg/mL. FIG. 12A depicts the number of colony forming units per 10⁻⁶ g of feces observed on LB agar plates with kanamycin was similar for the treatment group (E. coli with hm-NAS gene, N=6 mouse stool samples) and the control group (E. coli with empty vector, N=8 mouse stool samples). FIG. 12B depicts results demonstrating that in the antibiotic treated mouse cohort there are other colonizing bacteria present. Stool samples produced threefold more colony forming units on unselected LB agar plates compared to LB agar plates with kanamycin. Error bars are mean+/−SEM. In both cases when random colonies were picked from the LB/kanamycin plates they were all found to contain the cloning vector indicating these were in fact E. coli colonizing bacteria.

FIG. 13 depicts results identifying the N-acyl serinol synthase point mutant. LC-MS analysis of crude extracts prepared from cultures of E. coli expressing either the N-acyl serinol synthase gene or the N-acyl serinol synthase gene with an active site point mutation (E94A). N-acyl serinol metabolites (e.g., N-palmitoyl serinol and N-oleoyl serinol) are absent from the point mutant culture broth (ESI(+) mode). This mutant was created to address the possibility that the observed mouse phenotype might be due to over-production of any protein by E. coli and not specifically from N-acyl serinol production.

FIG. 14 depicts high-resolution reversed-phase LC-MS analysis of human fecal extract pooled from 128 samples representing 21 individuals. Extracted ion chromatograms for individual N-acyl amides are shown within a 2 ppm tolerance of the exact mass (M+H). Compounds observed to be present in the human fecal extract were confirmed by alignment to authentic standards (top panel), and by spiked addition of the pure compound (data not shown). No zwitterionic N-acyl amides (N-acyl or N-acyloxyacyl ornithine/lysines) were detected.

FIG. 15 depicts the NMR analysis of compound 2.

FIG. 16 depicts the ¹H NMR spectrum of compound 2 in DMSO-d₆.

FIG. 17 depicts the COSY spectrum of compound 2 in DMSO-d₆.

FIG. 18 depicts the HSQC spectrum of compound 2 in DMSO-d₆.

FIG. 19 depicts the HMBC spectrum of compound 2 in DMSO-d₆.

FIG. 20 depicts the HRESI-MS/MS fragmentation for compound 2.

FIG. 21 depicts the NMR analysis of compound 3.

FIG. 22 depicts the ¹H NMR spectrum of compound 3 in DMSO-d₆.

FIG. 23 depicts the ¹³C NMR spectrum of compound 3 in DMSO-d₆.

FIG. 24 depicts the COSY spectrum of compound 3 in DMSO-d₆.

FIG. 25 depicts the HSQC spectrum of compound 3 in DMSO-d₆.

FIG. 26 depicts the HMBC spectrum of compound 3 in DMSO-d₆.

FIG. 27 depicts the HRESI-MS/MS fragmentation of compound 3.

FIG. 28 depicts the NMR analysis of compound 4a.

FIG. 29 depicts the ¹H NMR spectrum of compound 4a in DMSO-d₆.

FIG. 30 depicts the ¹³C NMR spectrum of 4a in DMSO-d₆.

FIG. 31 depicts the COSY NMR spectrum of 4a in DMSO-d₆.

FIG. 32 depicts the HSQC spectrum of 4a in DMSO-d₆.

FIG. 33 depicts the HMBC spectrum of 4a in DMSO-d₆.

FIG. 34 depicts the NMR analysis of compound 4b.

FIG. 35 depicts the ¹H NMR spectrum of compound 4b in DMSO-d₆.

FIG. 36 depicts the COSY spectrum of compound 4b in DMSO-d₆.

FIG. 37 depicts the HMQC spectrum of compound 4b in DMSO-d₆.

FIG. 38 depicts the HMBC spectrum of compound 4b in DMSO-d₆.

FIG. 39 depicts the NMR analysis of compound 5.

FIG. 40 depicts the ¹H NMR spectrum of Compound 5 in DMSO-d₆.

FIG. 41 depicts the ¹³C NMR spectrum of Compound 5 in DMSO-d₆.

FIG. 42 depicts the COSY spectrum of Compound 5 in DMSO-d₆.

FIG. 43 depicts the HMQC spectrum of Compound 5 in DMSO-d₆.

FIG. 44 depicts the HMBC spectrum of Compound 5 in DMSO-d₆.

FIG. 45 depicts the NMR analysis of compound 6.

FIG. 46 depicts the ¹H NMR spectrum of Compound 6 in DMSO-d₆.

FIG. 47 depicts the ¹³C NMR spectrum of Compound 6 in DMSO-d₆.

FIG. 48 depicts the COSY spectrum of Compound 6 in DMSO-d₆.

FIG. 49 depicts the HMQC spectrum of Compound 6 in DMSO-d₆.

FIG. 50 depicts the HMBC spectrum of Compound 6 in DMSO-d₆.

### DETAILED DESCRIPTION

The present invention relates to compositions and methods for modulating G protein coupled receptors (GPCRs) to treat or prevent a disease or disorder.

In one embodiment, the composition of the invention comprises an N-acyl amide or a cell capable of producing an N-acyl amide. For example, in one embodiment, the invention provides a genetically engineered cell that expresses a human microbial N-acyl synthase (hm-NAS) gene.

In one embodiment, the method of the present invention comprises modulating a GPCR activity. In one embodiment, the method comprises administering to the subject an effective amount of a composition comprising at least one of a cell expressing an hm-NAS gene, an hm-NAS gene or an N-acyl amide. For example, in one embodiment, the methods modulate the activity of GPR119, SPR4, G2A, PTGIR, or PTGER4.

In one embodiment, the method of the present invention comprises treating or preventing a disease or disorder. In some embodiments, the disease or disorder is associated with abnormal GPCR activity. In one embodiment, the method comprises administering to the subject a therapeutically effective amount of a composition comprising an effective amount of a composition comprising at least hm-NAS gene or a N-acyl amide, cell expressing an hm-NAS gene.

Exemplary diseases or disorders treated or prevented by the compositions and methods of the invention include diabetes, obesity, colitis, autoimmune disorder, atherosclerosis, gastrophoresis, cirrhosis, non alcoholic fatty liver disease, non alcoholic steatohepatitis, inflammatory bowel disease, osteoporosis, and osteopenia.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element. “About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

As used herein, the term “alkyl,” by itself or as part of another substituent means, unless otherwise stated, a straight or branched chain hydrocarbon having the number of carbon atoms designated (i.e. C₁₋₆ means one to six carbon atoms) and includes straight, branched chain, or cyclic substituent groups. Examples include methyl, ethyl, propyl, isopropyl, butyl, isobutyl, tert-butyl, pentyl, neopentyl, hexyl, and cyclopropylmethyl. Most preferred is (C₁-C₆)alkyl, particularly ethyl, methyl, isopropyl, isobutyl, n-pentyl, n-hexyl and cyclopropylmethyl.

The term “alkenyl” as used herein contemplates both straight and branched chain alkene radicals. Preferred alkenyl groups are those containing two to fifteen carbon atoms. Additionally, the alkenyl group may be optionally substituted.

As used herein, the term “substituted” means that an atom or group of atoms has replaced hydrogen as the substituent attached to another group.

“Non-pathogenic bacteria” refer to bacteria that are not capable of causing disease or harmful responses in a host. In some embodiments, non-pathogenic bacteria are commensal bacteria. Examples of non-pathogenic bacteria include, but are not limited to Bacillus, Bacteroides, Bifidobacterium, Brevibacteria, Clostridium, Enterococcus, Escherichia coli, Lactobacillus, Lactococcus, Saccharomyces, and Staphylococcus, e.g., Bacillus coagulans, Bacillus subtilis, Bacteroides fragilis, Bacteroides subtilis, Bacteroides thetaiotaomicron, Bifidobacterium bifidum, Bifidobacterium infantis, Bifidobacterium lactis, Bifidobacterium longum, Clostridium butyricum, Enterococcus faecium, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, and Saccharomyces boulardii (Sonnenborn et al., 2009; Dinleyici et al., 2014; U.S. Pat. Nos. 6,835,376; 6,203,797; 5,589,168; 7,731,976). Naturally pathogenic bacteria may be genetically engineered to provide reduce or eliminate pathogenicity.

“Probiotic” is used to refer to live, non-pathogenic microorganisms, e.g., bacteria, which can confer health benefits to a host organism that contains an appropriate amount of the microorganism. In some embodiments, the host organism is a mammal. In some embodiments, the host organism is a human. Some species, strains, and/or subtypes of non-pathogenic bacteria are currently recognized as probiotic bacteria. Examples of probiotic bacteria include, but are not limited to, Bifidobacteria, Escherichia coli, Lactobacillus, and Saccharomyces, e.g., Bifidobacterium bifidum, Enterococcus faecium, Escherichia coli strain Nissle, Lactobacillus acidophilus, Lactobacillus bulgaricus, Lactobacillus paracasei, Lactobacillus plantarum, and Saccharomyces boulardii (Dinleyici et al., 2014; U.S. Pat. Nos. 5,589,168; 6,203,797; 6,835,376). The probiotic may be a variant or a mutant strain of bacterium (Arthur et al., 2012; Cuevas-Ramos et al., 2010; Olier et al., 2012; Nougayrede et al., 2006). Non-pathogenic bacteria may be genetically engineered to enhance or improve desired biological properties, e.g., survivability. Non-pathogenic bacteria may be genetically engineered to provide probiotic properties. Probiotic bacteria may be genetically engineered to enhance or improve probiotic properties.

As used herein, a “prebiotic” is a selectively fermented ingredient that allows specific changes, both in the composition and/or activity in the gastrointestinal microflora, which confers benefits upon host well-being and health.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

An “effective amount” or “therapeutically effective amount” of a compound is that amount of a compound which is sufficient to provide a beneficial effect to the subject to which the compound is administered. An “effective amount” of a delivery vehicle is that amount sufficient to effectively bind or deliver a compound.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The terms “patient,” “subject,” “individual,” and the like are used interchangeably herein, and refer to any animal, or cells thereof whether in vitro or in vivo, amenable to the methods described herein. In certain non-limiting embodiments, the patient, subject or individual is a human.

A “therapeutic” treatment is a treatment administered to a subject who exhibits signs or symptoms of pathology, for the purpose of diminishing or eliminating those signs or symptoms.

As used herein, “treating a disease or disorder” means reducing the severity and/or frequency with which a sign or symptom of the disease or disorder is experienced by a patient.

The term “expression” as used herein is defined as the transcription and/or translation of a particular nucleotide sequence driven by its promoter.

The term “expression vector” as used herein refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules, siRNA, ribozymes, and the like. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well.

“Isolated” means altered or removed from the natural state. For example, a nucleic acid or a peptide naturally present in its normal context in a living animal is not “isolated,” but the same nucleic acid or peptide partially or completely separated from the coexisting materials of its natural context is “isolated.” An isolated nucleic acid or protein can exist in substantially purified form, or can exist in a non-native environment such as, for example, a host cell.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared ×100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil). The term “nucleic acid” typically refers to large polynucleotides.

By “expression cassette” is meant a nucleic acid molecule comprising a coding sequence operably linked to promoter/regulatory sequences necessary for transcription and, optionally, translation of the coding sequence.

The term “operably linked” as used herein refer to the linkage of nucleic acid sequences in such a manner that a nucleic acid molecule capable of directing the transcription of a given gene and/or the synthesis of a desired protein molecule is produced. The term also refers to the linkage of sequences encoding amino acids in such a manner that a functional (e.g., enzymatically active, capable of binding to a binding partner, capable of inhibiting, etc.) protein or polypeptide is produced.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a n inducible manner.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention is based, in part, on the unexpected discovery of human microbial N-acyl synthase (hm-NAS) genes that produce N-acyl amides and that the N-acyl amides modulate the activity of G protein-coupled receptors (GPCRs). Accordingly, in some aspects the invention provides compositions and methods for treating diseases and disorders associated with abnormal GPCR activity.

In one embodiment, the invention provides a genetically engineered cell that expresses an hm-NAS gene. For example, in one embodiment, the invention provides a bacterial cell that is genetically engineered to express N-acyl serinol synthase. In one embodiment, the invention provides a composition comprising the genetically engineered cell.

The present invention also provides a method for modulating a GPCR. In one embodiment, the method comprises administering to the subject an effective amount of a composition comprising a cell genetically engineered to expresses a human microbial N-acyl synthase (hm-NAS) gene, an hm-NAS gene, or a N-acyl amide. In one embodiment, the GPCRs modulated by the methods of the invention are enriched in the gastrointestinal mucosa. For example, in one embodiment the GPCR is GPR119, SPR4, G2A, PTGIR, or PTGER4.

The present invention also provides a method for treating a disease or disorder in a subject. In one embodiment the method comprises administering to the subject a therapeutically effective amount of a composition comprising a cell genetically engineered to expresses a human microbial N-acyl synthase (hm-NAS) gene, an hm-NAS gene, or an N-acyl amide. In some embodiments, the method treats or prevents a disease or disorder associated with abnormal GPCR activity. For example, in one embodiment exemplary diseases and disorders treated or prevented by methods of the invention include diabetes, obesity, colitis, autoimmune disorder, atherosclerosis, gastrophoresis, cirrhosis, non-alcoholic fatty liver disease, non-alcoholic steatohepatitis, inflammatory bowel disease, osteoporosis, and osteopenia. In some embodiments, the disease or disorder is associated with abnormal gastric emptying, appetite, or glucose homeostasis.

### Cells

In one aspect, the present invention provides an engineered cell capable of producing an N-acyl amide. The genetically modified cell according to the invention may be constructed from any suitable host cell. The host cell may be an unmodified cell or may already be genetically modified. The cell may be a prokaryote cell, a eukaryote cell, a plant cell or an animal cell.

In one embodiment, the engineered cell is modified by way of introducing genetic material into the cell in order for the cell to increase production of an N-acyl amide. In some embodiments, the engineered cell produces an N-acyl amide, but not an N-acyl precursor.

In one embodiment, the engineered cell is modified by way of introducing a stimulus to the cell in order for the cell to increase production of an N-acyl amide. In one embodiment, the stimulus can be an agent including but not limited to a small molecule, a peptide, and the like.

In one embodiment, the cell is a eukaryotic cell. In one embodiment, the cell may be a human cell, a non-human mammalian cell, a non-mammalian vertebrate cell, an invertebrate cell, an insect cell, a plant cell, a yeast cell, or a single cell eukaryotic organism. In one embodiment, the cell may be an adult cell or an embryonic cell (e.g., an embryo). In one embodiment, the cell may be a stem cell. Suitable stem cells include without limit embryonic stem cells, ES-like stem cells, fetal stem cells, adult stem cells, pluripotent stem cells, induced pluripotent stem cells, multipotent stem cells, oligopotent stem cells, unipotent stem cells and others.

In one embodiment, the cell is a cell line cell. Non-limiting examples of suitable mammalian cells include Chinese hamster ovary (CHO) cells, baby hamster kidney (BHK) cells; mouse myeloma NS0 cells, mouse embryonic fibroblast 3T3 cells (NIH3T3), mouse B lymphoma A20 cells; mouse melanoma B16 cells; mouse myoblast C2C12 cells; mouse myeloma SP2/0 cells; mouse embryonic mesenchymal C3H-10T1/2 cells; mouse carcinoma CT26 cells, mouse prostate DuCuP cells; mouse breast EMT6 cells; mouse hepatoma Hepa1c1c7 cells; mouse myeloma J5582 cells; mouse epithelial MTD-1A cells; mouse myocardial MyEnd cells; mouse renal RenCa cells; mouse pancreatic RIN-5F cells; mouse melanoma X64 cells; mouse lymphoma YAC-1 cells; rat glioblastoma 9L cells; rat B lymphoma RBL cells; rat neuroblastoma B35 cells; rat hepatoma cells (HTC); buffalo rat liver BRL 3A cells; canine kidney cells (MDCK); canine mammary (CMT) cells; rat osteosarcoma D17 cells; rat monocyte/macrophage DH82 cells; monkey kidney SV-40 transformed fibroblast (COS 7) cells; monkey kidney CVI-76 cells; African green monkey kidney (VERO-76) cells; human embryonic kidney cells (HEK293, HEK293T); human cervical carcinoma cells (HELA); human lung cells (W138); human liver cells (Hep G2); human U2-OS osteosarcoma cells, human A549 cells, human A-431 cells, human SW48 cells, human HCT116 cells, and human K562 cells. An extensive list of mammalian cell lines may be found in the American Type Culture Collection catalog (ATCC, Manassas, Va.).

In one embodiment, the cell can be a prokaryotic cell or a eukaryotic cell. In one embodiment, the cell is a prokaryotic cell. In one embodiment, the cell is a genetically engineered bacteria cell.

In one embodiment, the genetically engineered bacteria cell is a non-pathogenic bacteria cell. In some embodiments, the genetically engineered bacteria cell is a commensal bacteria cell. In some embodiments, the genetically engineered bacteria cell is a probiotic bacteria cell. In some embodiments, the genetically engineered bacteria cell is a naturally pathogenic bacteria cell that is modified or mutated to reduce or eliminate pathogenicity. Exemplary bacteria include, but are not limited to Bacillus, Bacteroides, Bifidobacterium, Brevibacteria, Clostridium, Enterococcus, Escherichia coli, Lactobacillus, Lactococcus, Saccharomyces, and Staphylococcus, e.g., Bacillus coagulans, Bacillus subtilis, Bacteroides fragilis, Bacteroides subtilis, Bacteroides thetaiotaomicron, Bifidobacterium bifidum, Bifidobacterium infantis, Bifidobacterium lactis, Bifidobacterium longum, Clostridium butyricum, Enterococcus faecium, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, Lactococcus lactis, and Saccharomyces boulardii.

In some embodiments, the genetically engineered bacteria are Escherichia coli strain Nissle 1917 (E. coli Nissle), a Gram-negative bacterium of the Enterobacteriaceae family that “has evolved into one of the best characterized probiotics” (Ukena et al., 2007). The strain is characterized by its complete harmlessness (Schultz, 2008), and has GRAS (generally recognized as safe) status (Reister et al., 2014, emphasis added). Genomic sequencing confirmed that E. coli Nissle lacks prominent virulence factors (e.g., E. coli α-hemolysin, P-fimbrial adhesins) (Schultz, 2008). In addition, it has been shown that E. coli Nissle does not carry pathogenic adhesion factors, does not produce any enterotoxins or cytotoxins, is not invasive, and not uropathogenic (Sonnenborn et al., 2009). As early as in 1917, E. coli Nissle was packaged into medicinal capsules, called Mutaflor, for therapeutic use. E. coli Nissle has since been used to treat ulcerative colitis in humans in vivo (Rembacken et al., 1999), to treat inflammatory bowel disease, Crohn's disease, and pouchitis in humans in vivo (Schultz, 2008), and to inhibit enteroinvasive Salmonella, Legionella, Yersinia, and Shigella in vitro (Altenhoefer et al., 2004). It is commonly accepted that E. coli Nissle's therapeutic efficacy and safety have convincingly been proven (Ukena et al., 2007).

One of ordinary skill in the art would appreciate that the genetic modifications disclosed herein may be modified and adapted for other species, strains, and subtypes of bacteria.

### Genetic Modification

In one aspect, the present invention provides a cell genetically engineered to produce an N-acyl amide. In one embodiment, the genetically engineered cell expresses a human microbial NAS (hm-NAS) gene.

In one embodiment, the cells of the invention can be genetically modified, e.g., to express exogenous (e.g., introduced) genes (“transgenes”) or to repress the expression of endogenous genes, and the invention provides a method of genetically modifying such cells and populations. Preferably, the cells of the invention are genetically modified to express an hm-NAS gene. In accordance with this method, the cell is exposed to a gene transfer vector comprising a nucleic acid including an hm-NAS gene, such that the nucleic acid is introduced into the cell under conditions appropriate for the hm-NAS gene to be expressed within the cell. The hm-NAS gene generally is an expression cassette, including a polynucleotide operably linked to a suitable promoter. The polynucleotide can encode a protein, or it can encode biologically active RNA (e.g., antisense RNA or a ribozyme). Of course, where it is desired to employ gene transfer technology to deliver a given hm-NAS gene, its sequence will be known.

In one embodiment, the hm-NAS gene is able to produce an N-acyl amide. For example, in some embodiments, the hm-NAS gene is able to produce a N-acyl amide include, but not limited to, an N-acyl glycine, an N-acyloxyacyl lysine/ornithine, an N-acyloxyacyl glutamine, an N-acyl lysine/ornithine, an N-acyl alanine, or an N-acyl serinol.

In one embodiment, the hm-NAS gene is associated with the N-acyl synthase protein family PFAM13444. Exemplary hm-NAS genes include, but are not limited to genes identified in table 1 and table 2.

TABLE 1
heterologously expressed hm-NAS genes

| Clone Number | EBI Gene | Gene Size (bp) | Molecule Family |
|---|---|---|---|
| 1 | EFI7261 | 1191 | No production |
| 2 | EHB91285 | 921 | 1 |
| 3 | EEK17761 | 960 | No production |
| 5 | EEY82825 | 987 | 1 |
| 6 | EHP49568 | 969 | No production |
| 7 | EHG23013 | 1008 | 1 |
| 8 | EFA42931 | 999 | 1 |
| 9 | EFL47029 | 1005 | 1 |
| 10 | EHO75052 | 1005 | 1 |
| 11 | ADK95845 | 1011 | 1 |
| 12 | EFV04460 | 1017 | 1 |
| 13 | EHH01788 | 945 | 1 |
| 14 | EDY97076 | 1002 | 1 |
| 15 | CBW20928 | 1026 | 1 |
| 16 | EDS14876 | 1035 | 1 |
| 17 | EDO52243 | 990 | 1 |
| 18 | CBK67812 | 1029 | 1 |
| 19 | ACI09609 | 1713 | 3 |
| 21 | ABV66681 | 1716 | 2 |
| 24 | EHT12133 | 1731 | 2 |
| 26 | EFE54303 | 1743 | 2 |
| 27 | EFE94777 | 1734 | 2 |
| 29 | EER56350 | 768 | No production |
| 30 | EET45812 | 783 | 4 |
| 31 | ACS62992 | 846 | 4 |
| 33 | BAH33083 | 849 | No production |
| 35 | EFG73978 | 870 | No production |
| 36 | CAW29482 | 768 | 4 |
| 37 | EFH13337 | 813 | 4 |
| 38 | EGP09383 | 1041 | No production |
| 39 | EEV22085 | 1011 | No production |
| 40 | EEY94333 | 789 | No production |
| 41 | EFF83269 | 789 | No production |
| 42 | CAP01857 | 816 | 4 |
| 43 | EGP10046 | 804 | 5 |
| 50 | EFK33376 | 1854 | No production |
| 51 | EEK14630 | 1815 | No production |
| 52 | EFS97491 | 1848 | 2 |
| 53 | CBK85930 | 1713 | 2 |
| 54 | EHM48796 | 1713 | 2 |
| 55 | EEK89350 | 1596 | No production |
| 56 | EHL05550 | 1638 | 6 |
| 57 | EFV76279 | 1623 | 6 |
| 58 | GL883582 | 1576 | 6 |

Molecule Family 1 - N-acyl glycines
Molecule Family 2 - N¬-acyloxyacyl lysine
Molecule Family 3 - N-acyloxyacyl glutamine
Molecule Family 4 - N-acyl lysine/ornithine
Molecule Family 5 - N-acyl alanine
Molecule Family 6 - N-acyl serinols

TABLE 2
PFAM13444 related hm-NAS genes

| PFAM13444 Gene | N-acyl Amide Molecule | | Related Human Microbial Gene |
|---|---|---|---|
| EBI reference information | Family | E-Value | Identified in HMP reference genome |
| R6A3N1_9BACT/51-156 | 1 | 2.00E−22 | >ADDV01000044 Prevotella oris C735 |
| R6EH40_9BACT/51-155 | 1 | 3.00E−74 | >ADDV01000044 Prevotella oris C735 |
| R7PBT6_9BACT/52-156 | 1 | 6.00E−07 | >ADCT01000041 Prevotella sp. C561 |
| R7NN97_9BACE/51-155 | 1 | 0 | >AQHY01000032 Bacteroides |
| | | | massiliensis B84634 |
| A0A0C3RD59_9PORP/51- | 1 | 4.00E−13 | >GG705232 Bacteroides sp. |
| 157 | | | 3_1_33FAA |
| A6L081_BACV8/51-155 | 1 | 0 | >ADKO01000098 Bacteroides |
| | | | vulgatus PC510 |
| A6LEV2_PARD8/51-155 | 1 | 0 | >ACPW01000045 Parabacteroides sp. |
| | | | D13 |
| D4IM11_9BACT/57-158 | 1 | 0 | >ADKO01000098 Bacteroides |
| | | | vulgatus PC510 |
| D5EVS3_PRER2/52-157 | 1 | 2.00E−126 | >DS995534 Bacteroides dorei DSM |
| | | | 17855 |
| D6D060_9BACE/51-155 | 1 | 0 | >GG705232 Bacteroides sp. |
| | | | 3_1_33FAA |
| E6SVI0_BACT6/51-155 | 1 | 0 | >FP929032 Alistipes shahii WAL 8301 |
| CBK67812_CBK67812.1_Bacteroides_xylanisolvens_XB1A_hypothetical_protein | 1 | 0 | >GG703854 Prevotella copri DSM |
| | | | 18205 |
| ENA_CBW20928_CBW20928.1_Bacteroides_fragilis_638R_putative_hemolysin_A | 1 | 0 | >FP929033 Bacteroides xylanisolvens |
| | | | XB1A |
| ENA_EDO52243_EDO52243.1_Bacteroides_uniformis_ATCC_8492_hemolysin | 1 | 0 | >GL882689 Bacteroides fluxus YIT |
| | | | 12057 |
| ENA_EDS14876_EDS14876.1_Bacteroides_stercoris_ATCC_43183_hemolysin_{—} | 1 | 0 | >FP929033 Bacteroides xylanisolvens |
| | | | XB1A |
| ENA_EDY97076_EDY97076.1_Bacteroides_plebeius_DSM_17135_hemolysin_{—} | 1 | 0 | >JH636044 Bacteroides sp. 3_2_5 |
| ENA_EEY82825_EEY82825.1_Bacteroides_sp._2_1_33B_hemolysin_{—} | 1 | 0 | >ACPT01000029 Bacteroides sp. D20 |
| ENA_EFV04460_EFV04460.1_Prevotella_salivae_DSM_15606_hemolysin_{—} | 1 | 0 | >ABFZ02000020 Bacteroides stercoris |
| | | | ATCC 43183 |
| ENA_EHB91285_EHB91285.1 Alistipes_indistinctus_YIT_12060_hypothetical_protein_{—} | 1 | 0 | >ABQC02000004 Bacteroides plebeius |
| | | | DSM 17135 |
| ENA_EHH01788_EHH01788.1_Paraprevotella_clara_YIT_11840_hemolysin | 1 | 0 | >GG705151 Bacteroides sp. 2_1_33B |
| ENA_EHP49568_EHP49568.1_Odoribacter_laneus_YIT_12061_hypothetical_protein | 1 | 0 | >GL629647 Prevotella salivae DSM |
| | | | 15606 |
| I3YLB0_ALIFI/56-157 | 1 | 0 | >JH370372 Alistipes indistinctus YIT |
| | | | 12060 |
| Q5LII1_BACFN/51-155 | 1 | 0 | >JH376579 Paraprevotella clara YIT |
| | | | 11840 |
| Q8A247_BACTN/51-155 | 1 | 0 | >JH594596 Odoribacter laneus YIT |
| | | | 12061 |
| R5C642_9BACE/51-155 | 1 | 8.00E−120 | >FP929032 Alistipes shahii WAL 8301 |
| R5FQF1_9BACT/53-157 | 1 | 1.00E−113 | >ACWI01000002 Bacteroides sp. |
| | | | 2_1_56FAA |
| R5I942_9PORP/51-156 | 1 | 5.00E−22 | >JH636041 Bacteroides sp. 1_1_6 |
| R5JGR8_9BACE/51-155 | 1 | 0 | >KB905466 Bacteroides salyersiae |
| | | | WAL 10018 |
| R5KD71_9BACT/52-157 | 1 | 6.00E−171 | >GL629647 Prevotella salivae DSM |
| | | | 15606 |
| R5MMX8_9BACE/51-155 | 1 | 0 | >ACWH01000030 Bacteroides ovatus |
| | | | 3_8_47FAA |
| R5NZI1_9BACT/51-155 | 1 | 0 | >KB905466 Bacteroides salyersiae |
| | | | WAL 10018 |
| R5UEV5_9BACE/51-155 | 1 | 0 | >JH379426 Prevotella stercorea DSM |
| | | | 18206 |
| R5UPI5_9PORP/51-157 | 1 | 0 | >ABJL02000006 Bacteroides |
| | | | intestinalis DSM 17393 |
| R5VW07_9BACE/51-155 | 1 | 0 | >JH376579 Paraprevotella clara YIT |
| | | | 11840 |
| R6B4U0_9BACT/52-156 | 1 | 0 | >AAVM02000009 Bacteroides caccae |
| | | | ATCC 43185 |
| R6BXV9_9BACT/52-157 | 1 | 0 | >GG703854 Prevotella copri DSM |
| | | | 18205 |
| R6DH15_9BACE/51-155 | 1 | 0 | >GG688329 Bacteroides finegoldii |
| | | | DSM 17565 |
| R6FKP1_9BACE/51-155 | 1 | 0 | >DS499674 Bacteroides stercoris |
| | | | ATCC 43183 |
| R6FUQ8_9BACT/52-158 | 1 | 0 | >JH379426 Prevotella stercorea DSM |
| | | | 18206 |
| R6KTM3_9BACE/51-155 | 1 | 0 | >ACCH01000127 Bacteroides |
| | | | cellulosilyticus DSM 14838 |
| R6LNJ9_9BACE/51-154 | 1 | 0 | >AFBM01000001 Bacteroides clarus |
| | | | YIT 12056 |
| R6MX16_9BACE/51-155 | 1 | 0 | >DS981492 Bacteroides coprocola |
| | | | DSM 17136 |
| R6QE29_9BACT/52-157 | 1 | 0 | >GG703854 Prevotella copri DSM |
| | | | 18205 |
| R6S950_9BACE/51-155 | 1 | 0 | >GG688329 Bacteroides finegoldii |
| | | | DSM 17565 |
| R6SC61_9BACE/51-155 | 1 | 0 | >ACBW01000097 Bacteroides |
| | | | coprophilus DSM 18228 |
| R6VUA1_9BACT/56-157 | 1 | 0 | >FP929032 Alistipes shahii WAL 8301 |
| R6XGV7_9BACT/52-157 | 1 | 6.00E−106 | >GG703854 Prevotella copri DSM |
| | | | 18205 |
| R6YIB5_9BACE/51-155 | 1 | 2.00E−121 | >ACTC01000036 Bacteroides sp. |
| | | | 4_1_36 |
| R7DDR3_9PORP/51-155 | 1 | 0 | >ACWX01000035 Tannerella sp. |
| | | | 6_1_58FAA_CT1 |
| R7EIP8_9BACE/51-155 | 1 | 0 | >ACPT01000029 Bacteroides sp. D20 |
| R7F021_9BACT/51-157 | 1 | 2.00E−11 | >AFZZ01000132 Prevotella stercorea |
| | | | DSM 18206 |
| R7HSG0_9BACT/37-143 | 1 | 2.00E−26 | >AFZZ01000132 Prevotella stercorea |
| | | | DSM 18206 |
| R7IYP9_9BACT/59-165 | 1 | 1.00E−58 | >JH379426 Prevotella stercorea DSM |
| | | | 18206 |
| R7JHM4_9BACT/51-152 | 1 | 0 | >ABFK02000017 Alistipes putredinis |
| | | | DSM 17216 |
| E6K481_9BACT/52-156 | 1 | 0 | >AEPD01000010 Prevotella buccae |
| | | | ATCC 33574 |
| ENA_ADK95845_ADK95845.1_Prevotella_melaninogenica_ATCC_25845_hemolysin_{—} | 1 | 0 | >CP002122 Prevotella melaninogenica |
| | | | ATCC 25845 |
| ENA_EFI17261_EFI17261.1_Bacteroidetes_oral_taxon_274_str._F0058_hemolysin | 1 | 0 | >ADCM01000011 Bacteroidetes oral |
| | | | taxon 274 str. F0058 |
| ENA_EHG23013_EHG23013.1_Alloprevotella_rava_F0323_hypothetical_protein | 1 | 0 | >JH376829 Prevotella sp. oral taxon |
| | | | 302 str. F0323 |
| ENA_EHO75052_EHO75052.1_Prevotella_micans_F0438_hypothetical_protein | 1 | 0 | >JH594521 Prevotella micans F0438 |
| F2KX19_PREDF/64-168 | 1 | 0 | >CP002589 Prevotella denticola F0289 |
| F9D3S1_PREDD/52-156_1 | 1 | 0 | >GL982488 Prevotella dentalis DSM |
| | | | 3688 |
| I1YUM9_PREI7/53-157 | 1 | 1.00E−98 | >GG703886 Prevotella oris F0302 |
| Q7MTR9_PORGI/53-158 | 1 | 0 | >AJZS01000078 Porphyromonas |
| | | | gingivalis W50 |
| R5CSR0_9BACT/52-157 | 1 | 3.00E−115 | >AWEY01000007 Prevotella baroniae |
| | | | F0067 |
| R5GFN8_9BACT/51-155 | 1 | 4.00E−29 | >ACZS01000081 Prevotella sp. oral |
| | | | taxon 472 str. F0295 |
| R5Q4D6_9BACT/52-157 | 1 | 6.00E−107 | >AWET01000051 Prevotella |
| | | | Pleuritidis F0068 |
| R6W2Q2_9BACT/52-156 | 1 | 3.00E−160 | >GL872283 Prevotella multiformis |
| | | | DSM 16608 |
| R7CYB8_9BACE/51-155 | 1 | 3.00E−15 | >CP002122 Prevotella melaninogenica |
| | | | ATCC 25845 |
| W0EP20_9PORP/51-155 | 1 | 5.00E−43 | >AWEY01000007 Prevotella baroniae |
| | | | F0067 |
| C7M608_CAPOD/352-453 | 2 | 0 | >AMEV01000023 Capnocytophaga sp. |
| | | | oral taxon 324 str. F0483 |
| ENA_EEK14630_EEK14630.1_Capnocytophaga_gingivalis_ATCC_33624_Acyltransferase_{—} | 2 | 0 | >ACLQ01000018 Capnocytophaga |
| | | | gingivalis ATCC 33624 |
| ENA_EFS97491_EFS97491.1_Capnocytophaga_ochracea_F0287_Acyltransferase | 2 | 0 | >AKFV01000035 Capnocytophaga |
| | | | ochracea str. Holt 25 |
| F9YU78_CAPCC/351-452 | 2 | 8.00E−173 | >AMEV01000023 Capnocytophaga sp. |
| | | | oral taxon 324 str. F0483 |
| H1Z9S5_MYROD/346-447 | 2 | 2.00E−40 | >ALNN01000028 Capnocytophaga sp. |
| | | | CM59 |
| ENA_EFA42931_EFA42931.1_Prevotella_bergensis_DSM_17361_hemolysin | 1 | 0 | >GG704783 Prevotella bergensis DSM |
| | | | 17361 |
| A0A095ZG93_9BACT/52- | 1 | 0 | >ADEG01000046 Prevotella buccalis |
| 156 | | | ATCC 35310 |
| E7RNE3_9BACT/52-156 | 1 | 0 | >AEPE02000002 Prevotella oralis |
| | | | ATCC 33269 |
| ENA_EEK17761_EEK17761.1_Porphyromonas_uenonis_60- | 1 | 0 | >ACLR01000009 Porphyromonas |
| 3_hemolysin_{—} | | | uenonis 60-3 |
| ENA_EFL47029_EFL47029.1_Prevotella_disiens_FB035- | 1 | 0 | >AEDO01000009 Prevotella disiens |
| 09AN_hemolysin_{—} | | | FB035-09AN |
| F4KL89_PORAD/55-160 | 1 | 0 | >AENO01000054 Porphyromonas |
| | | | asaccharolytica PR426713P-I |
| I4Z8L9_9BACT/52-156 | 1 | 0 | >ADFO01000053 Prevotella bivia |
| | | | JCVIHMP010 |
| R6CE12_9BACE/51-155 | 1 | 1.00E−11 | >AEDO01000009 Prevotella disiens |
| | | | FB035-09AN |
| R6XAK6_9BACT/52-156 | 1 | 1.00E−120 | >AEPE02000002 Prevotella oralis |
| | | | ATCC 33269 |
| ENA_EHL05550_EHL05550.1_Desulfitobacterium_hafniense_DP7_aminotransferase_class_V_{—} | 6 | 0 | >JH414482 Desulfitobacterium |
| | | | hafniense DP7 |
| ENA_EFV76279_EFV76279.1_Bacillus_sp._2_A_57_CT2_serine- | 6 | 0 | >GL635754 Bacillus sp. 2_A_57_CT2 |
| pyruvate_aminotransferase | | | |
| A6T596_KLEP7/322-423 | 2 | 0 | >JH930419 Klebsiella pneumoniae |
| | | | subsp. pneumoniae WGLW2 |
| D8MWX6_ERWBE/367- | 2 | 3.00E−147 | >GG753567 Serratia odorifera DSM |
| 468 | | | 4582 |
| ENA_EFE94777_EFE94777.1_Serratia_odorifera_DSM_4582_Acyltransferase | 2 | 0 | >GG753567 Serratia odorifera DSM |
| | | | 4582 |
| Q6CZN2_PECAS/322-423 | 2 | 2.00E−109 | >ADBY01000051 Serratia odorifera |
| | | | DSM 4582 |
| A0A0B5CH45_NEIEG/32- | 4 | 0 | >ADBF01000232 Neisseria elongata |
| 132 | | | subsp. glycolytica ATCC 29315 |
| E5UJR0_NEIMU/32-132 | 4 | 0 | >ACRG01000005 Neisseria mucosa |
| | | | C102 |
| ENA_EET45812_EET45812.1_Neisseria_sicca_ATCC_29256_hypothetical_protein | 4 | 0 | >ACKO02000002 Neisseria sicca |
| | | | ATCC 29256 |
| ENA_ACI09609_ACI09609.1_Klebsiella_pneumoniae_342_conserved_hypothetical_protein | 3 | 0 | >ACXA01000063 Klebsiella sp. |
| | | | 1_1_55 |
| A4W746_ENT38/322-423 | 2 | 0 | >FP929040 Enterobacter cloacae |
| | | | subsp. cloacae NCTC 9394 |
| ENA_CBK85930_CBK85930.1_Enterobacter_cloacae_subsp._cloacae_NCTC_9394_Putative_hemolysin_{—} | 2 | 0 | >FP929040 Enterobacter cloacae |
| | | | subsp. cloacae NCTC 9394 |
| ENA_EFE54303_EFE54303.1_Providencia_rettgeri_DSM_1131_Acyltransferase | 2 | 0 | >ACCI02000039 Providencia rettgeri |
| | | | DSM 1131 |
| ENA_EHM48796_EHM48796.1_Yokenella_regensburgei_ATCC_43003_Acyltransferase | 2 | 0 | >JH417874 Yokenella regensburgei |
| | | | ATCC 43003 |
| F9ZAJ4_ODOSD/341-443 | 2 | 0 | >JH594597 Odoribacter laneus YIT |
| | | | 12061 |
| G9Z3T1_9ENTR/322-423 | 2 | 0 | >JH417874 Yokenella regensburgei |
| | | | ATCC 43003 |
| R5UYM1_9PORP/338-439 | 2 | 0 | >ADMC01000028 Odoribacter laneus |
| | | | YIT 12061 |
| ENA_ACS62992_ACS62992.1_Ralstonia_pickettii_12D_conserved_hypothetical_protein_{—} | 4 | 0 | >GL520222 Ralstonia sp. 5_7_47FAA |
| ENA_CAW29482_CAW29482.1_Pseudomonas_aeruginosa_LESB58_putative_hemolysin_{—} | 4 | 0 | >ACWU01000206 Pseudomonas sp. |
| | | | 2_1_26 |
| A0A089UDH2_9ENTR/323- | 2 | 0 | >ALNJ01000086 Klebsiella sp. |
| 424 | | | OBRC7 |
| E6WAC8_PANSA/322-423 | 2 | 7.00E−59 | >GL892086 Enterobacter hormaechei |
| | | | ATCC 49162 |
| ENA_EHT12133_EHT12133.1_Raoultella_ornithinolytica_10- | 2 | 0 | >ALNJ01000086 Klebsiella sp. |
| 5246_hypothetical_protein | | | OBRC7 |
| G7LV45_9ENTR/322-423 | 2 | 5.00E−105 | >ALNJ01000086 Klebsiella sp. |
| | | | OBRC7 |
| ENA_EER56350_EER56350.1_Neisseria_flavescens_SK114_hypothetical_protein_{—} | 4 | 0 | >ACQV01000022 Neisseria flavescens |
| | | | SK114 |
| A0A077KL19_9FLAO/353- | 2 | 0 | >GL379781 Chryseobacterium gleum |
| 454 | | | ATCC 35910 |
| A7MLT3_CROS8/322-423 | 2 | 1.00E−178 | >AMLL01000012 Klebsiella |
| | | | pneumoniae subsp. pneumoniae |
| | | | WGLW1 |
| ENA_EFK33376_EFK33376.1_Chryseobacterium_gleum_ATCC_35910_Acyltransferase_{—} | 2 | 0 | >GL379781 Chryseobacterium gleum |
| | | | ATCC 35910 |
| ENA_CAP01857_CAP01857.2_Acinetobacter_baumannii_SDF_conserved_hypothetical_protein_{—} | 4 | 0 | >ACQB01000026 Acinetobacter |
| | | | baumannii ATCC 19606 |

In one embodiment, the cell expresses the hm-NAS gene N-acyl serinol synthase.

In one embodiment, the hm-NAS gene encodes for a protein comprising an amino acid sequence of an hm-NAS protein selected from hm-NAS proteins listed in table 1 and table 2.

In one embodiment, the hm-NAS gene comprises a nucleic acid sequence selected from the nucleic acid of an hm-NAS gene selected from hm-NAS genes of listed in table 1 and table 2.

The invention should also be construed to include any form of a gene having substantial homology to an hm-NAS gene. Preferably, a gene which is “substantially homologous” is about 50% homologous, more preferably about 70% homologous, even more preferably about 80% homologous, more preferably about 90% homologous, even more preferably, about 95% homologous, and even more preferably about 99% homologous to the hm-NAS gene.

In the context of gene therapy, the cells of the invention can be treated with a gene of interest prior to delivery of the cells into the recipient. In some cases, such cell-based gene delivery can present significant advantages of other means of gene delivery, such as direct injection of an adenoviral gene delivery vector. Delivery of a therapeutic gene that has been pre-inserted into cells avoids the problems associated with penetration of gene therapy vectors into desired cells in the recipient.

Accordingly, the invention provides the use of genetically modified cells that have been cultured according to the methods of the invention. Genetic modification may, for instance, result in the expression of an exogenous hm-NAS gene or in a change of expression of an endogenous hm-NAS gene. Such genetic modification may have therapeutic benefit. Genetic modification may also include at least a second gene. A second gene may encode, for instance, a selectable antibiotic-resistance gene or another selectable marker.

The cells of the invention may be genetically modified using any method known to the skilled artisan. See, for instance, Sambrook et al. (2012, Molecular Cloning: A Laboratory Manual, Cold Spring Harbor Laboratory Press, Cold Spring Harbor, N.Y.). For example, a cell may be exposed to an expression vector comprising a nucleic acid including a hm-NAS gene, such that the nucleic acid is introduced into the cell under conditions appropriate for the hm-NAS gene to be expressed within the cell. The hm-NAS gene generally is an expression cassette, including a polynucleotide operably linked to a suitable promoter. The polynucleotide can encode a protein, or it can encode biologically active RNA (e.g., antisense RNA or a ribozyme).

Nucleic acids can be of various lengths. Nucleic acid lengths typically range from about 20 nucleotides to 20 Kb, or any numerical value or range within or encompassing such lengths, 10 nucleotides to 10 Kb, 1 to 5 Kb or less, 1000 to about 500 nucleotides or less in length. Nucleic acids can also be shorter, for example, 100 to about 500 nucleotides, or from about 12 to 25, 25 to 50, 50 to 100, 100 to 250, or about 250 to 500 nucleotides in length, or any numerical value or range or value within or encompassing such lengths. Shorter polynucleotides are commonly referred to as “oligonucleotides” or “probes” of single- or double-stranded DNA.

Nucleic acids can be produced using various standard cloning and chemical synthesis techniques. Techniques include, but are not limited to nucleic acid amplification, e.g., polymerase chain reaction (PCR), with genomic DNA or cDNA targets using primers (e.g., a degenerate primer mixture) capable of annealing to antibody encoding sequence. Nucleic acids can also be produced by chemical synthesis (e.g., solid phase phosphoramidite synthesis) or transcription from a gene. The sequences produced can then be translated in vitro, or cloned into a plasmid and propagated and then expressed in a cell (e.g., a host cell such as yeast or bacteria, a eukaryote such as an animal or mammalian cell or in a plant).

Nucleic acids can be included within vectors as cell transfection typically employs a vector. The term “vector,” refers to, e.g., a plasmid, virus, such as a viral vector, or other vehicle known in the art that can be manipulated by insertion or incorporation of a polynucleotide, for genetic manipulation (i.e., “cloning vectors”), or can be used to transcribe or translate the inserted polynucleotide (i.e., “expression vectors”). Such vectors are useful for introducing polynucleotides in operable linkage with a nucleic acid, and expressing the transcribed encoded protein in cells in vitro, ex vivo or in vivo.

A vector generally contains at least an origin of replication for propagation in a cell. Control elements, including expression control elements, present within a vector, are included to facilitate transcription and translation. The term “control element” is intended to include, at a minimum, one or more components whose presence can influence expression, and can include components other than or in addition to promoters or enhancers, for example, leader sequences and fusion partner sequences, internal ribosome binding sites (IRES) elements for the creation of multigene, or polycistronic, messages, splicing signal for introns, maintenance of the correct reading frame of the gene to permit in-frame translation of mRNA, polyadenylation signal to provide proper polyadenylation of the transcript of a gene of interest, stop codons, among others.

Vectors included are those based on viral vectors, such as retroviral (lentivirus for infecting dividing as well as non-dividing cells), foamy viruses (U.S. Pat. Nos. 5,624,820, 5,693,508, 5,665,577, 6,013,516 and 5,674,703; WO92/05266 and WO92/14829), adenovirus (U.S. Pat. Nos. 5,700,470, 5,731,172 and 5,928,944), adeno-associated virus (AAV) (U.S. Pat. No. 5,604,090), herpes simplex virus vectors (U.S. Pat. No. 5,501,979), cytomegalovirus (CMV) based vectors (U.S. Pat. No. 5,561,063), reovirus, rotavirus genomes, simian virus 40 (SV40) or papilloma virus (Cone et al., Proc. Natl. Acad. Sci. USA 81:6349 (1984); Eukaryotic Viral Vectors, Cold Spring Harbor Laboratory, Gluzman ed., 1982; Sarver et al., Mol. Cell. Biol. 1:486 (1981); U.S. Pat. No. 5,719,054). Adenovirus efficiently infects slowly replicating and/or terminally differentiated cells and can be used to target slowly replicating and/or terminally differentiated cells. Simian virus 40 (SV40) and bovine papilloma virus (BPV) have the ability to replicate as extra-chromosomal elements (Eukaryotic Viral Vectors, Cold Spring Harbor Laboratory, Gluzman ed., 1982; Sarver et al., Mol. Cell. Biol. 1:486 (1981)). Additional viral vectors useful for expression include reovirus, parvovirus, Norwalk virus, coronaviruses, paramyxo- and rhabdoviruses, togavirus (e.g., sindbis virus and semliki forest virus) and vesicular stomatitis virus (VSV) for introducing and directing expression of a polynucleotide or transgene in pluripotent stem cells or progeny thereof (e.g., differentiated cells).

Vectors including a nucleic acid can be expressed when the nucleic acid is operably linked to an expression control element. As used herein, the term “operably linked” refers to a physical or a functional relationship between the elements referred to that permit them to operate in their intended fashion. Thus, an expression control element “operably linked” to a nucleic acid means that the control element modulates nucleic acid transcription and as appropriate, translation of the transcript.

The term “expression control element” refers to nucleic acid that influences expression of an operably linked nucleic acid. Promoters and enhancers are particular non-limiting examples of expression control elements. A “promoter sequence” is a DNA regulatory region capable of initiating transcription of a downstream (3′ direction) sequence. The promoter sequence includes nucleotides that facilitate transcription initiation. Enhancers also regulate gene expression, but can function at a distance from the transcription start site of the gene to which it is operably linked. Enhancers function at either 5′ or 3′ ends of the gene, as well as within the gene (e.g., in introns or coding sequences). Additional expression control elements include leader sequences and fusion partner sequences, internal ribosome binding sites (IRES) elements for the creation of multigene, or polycistronic, messages, splicing signal for introns, maintenance of the correct reading frame of the gene to permit in-frame translation of mRNA, polyadenylation signal to provide proper polyadenylation of the transcript of interest, and stop codons.

Expression control elements include “constitutive” elements in which transcription of an operably linked nucleic acid occurs without the presence of a signal or stimuli. For expression in mammalian cells, constitutive promoters of viral or other origins may be used. For example, SV40, or viral long terminal repeats (LTRs) and the like, or inducible promoters derived from the genome of mammalian cells (e.g., metallothionein IIA promoter; heat shock promoter, steroid/thyroid hormone/retinoic acid response elements) or from mammalian viruses (e.g., the adenovirus late promoter; mouse mammary tumor virus LTR) are used.

Expression control elements that confer expression in response to a signal or stimuli, which either increase or decrease expression of operably linked nucleic acid, are “regulatable.” A regulatable element that increases expression of operably linked nucleic acid in response to a signal or stimuli is referred to as an “inducible element.” A regulatable element that decreases expression of the operably linked nucleic acid in response to a signal or stimuli is referred to as a “repressible element” (i.e., the signal decreases expression; when the signal is removed or absent, expression is increased).

Expression control elements include elements active in a particular tissue or cell type, referred to as “tissue-specific expression control elements.” Tissue-specific expression control elements are typically more active in specific cell or tissue types because they are recognized by transcriptional activator proteins, or other transcription regulators active in the specific cell or tissue type, as compared to other cell or tissue types.

In accordance with the invention, there are provided cells and their progeny transfected with a nucleic acid or vector. Such transfected cells include but are not limited to a primary cell isolate, populations or pluralities of pluripotent stem cells, cell cultures (e.g., passaged, established or immortalized cell line), as well as progeny cells thereof (e.g., a progeny of a transfected cell that is clonal with respect to the parent cell, or has acquired a marker or other characteristic of differentiation).

The nucleic acid or protein can be stably or transiently transfected (expressed) in the cell and progeny thereof. The cell(s) can be propagated and the introduced nucleic acid transcribed and protein expressed. A progeny of a transfected cell may not be identical to the parent cell, since there may be mutations that occur during replication.

Viral and non-viral vector means of delivery into cells, in vitro, in vivo and ex vivo are included. Introduction of compositions (e.g., nucleic acid and protein) into the cells can be carried out by methods known in the art, such as osmotic shock (e.g., calcium phosphate), electroporation, microinjection, cell fusion, etc. Introduction of nucleic acid and polypeptide in vitro, ex vivo and in vivo can also be accomplished using other techniques. For example, a polymeric substance, such as polyesters, polyamine acids, hydrogel, polyvinyl pyrrolidone, ethylene-vinylacetate, methylcellulose, carboxymethylcellulose, protamine sulfate, or lactide/glycolide copolymers, polylactide/glycolide copolymers, or ethylenevinylacetate copolymers. A nucleic acid can be entrapped in microcapsules prepared by coacervation techniques or by interfacial polymerization, for example, by the use of hydroxymethylcellulose or gelatin-microcapsules, or poly (methylmethacrolate) microcapsules, respectively, or in a colloid system. Colloidal dispersion systems include macromolecule complexes, nano-capsules, microspheres, beads, and lipid-based systems, including oil-in-water emulsions, micelles, mixed micelles, and liposomes.

Liposomes for introducing various compositions into cells are known in the art and include, for example, phosphatidylcholine, phosphatidylserine, lipofectin and DOTAP (e.g., U.S. Pat. Nos. 4,844,904, 5,000,959, 4,863,740, and 4,975,282; and GIBCO-BRL, Gaithersburg, Md.). Piperazine based amphilic cationic lipids useful for gene therapy also are known (see, e.g., U.S. Pat. No. 5,861,397). Cationic lipid systems also are known (see, e.g., U.S. Pat. No. 5,459,127). Polymeric substances, microcapsules and colloidal dispersion systems such as liposomes are collectively referred to herein as “vesicles.”

The vectors of the present invention may also be used for gene therapy, using standard gene delivery protocols. Methods for gene delivery are known in the art. See, e.g., U.S. Pat. Nos. 5,399,346, 5,580,859, 5,589,466, incorporated by reference herein in their entireties. In another embodiment, the invention provides a gene therapy vector.

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

For example, vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. In one embodiment, the composition includes a vector derived from an adeno-associated virus (AAV). Adeno-associated viral (AAV) vectors have become powerful gene delivery tools for the treatment of various disorders. AAV vectors possess a number of features that render them ideally suited for gene therapy, including a lack of pathogenicity, minimal immunogenicity, and the ability to transduce postmitotic cells in a stable and efficient manner. Expression of a particular gene contained within an AAV vector can be specifically targeted to one or more types of cells by choosing the appropriate combination of AAV serotype, promoter, and delivery method

In certain embodiments, the vector also includes conventional control elements which are operably linked to the transgene in a manner which permits its transcription, translation and/or expression in a cell transfected with the plasmid vector or infected with the virus produced by the invention. As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

Probiotic Compositions In one aspect, the invention provides a probiotic composition which is capable of producing at least one N-acyl amide. In one embodiment, the probiotic composition is useful for modulating GPCR activity. In one embodiment, the probiotic composition is useful for treating or preventing a disease or disorder associated with abnormal GPCR activity.

In one embodiment, the probiotic composition is useful for modulating Peroxisome proliferator-activated receptor (PPAR) alpha activity. For example, in one embodiment, probiotic composition is useful for modulating the activity, expression or both of one or more of TRPV4, TRPA1 and SK3. In one embodiment, the probiotic composition is useful for treating or preventing a disease or disorder associated with abnormal PPAR activity. For example, in one embodiment the probiotic composition is useful for treating or preventing cirrhosis, fatty liver disease or inflammatory pain.

In one embodiment, the probiotic composition comprises a genetically engineered cell of the invention. For example, in one embodiment, the probiotic composition comprises a bacterial cell engineered to expresses a human microbial N-acyl synthase (hm-NAS) gene.

In one embodiment, the probiotic composition further comprises another microorganism. For example, in one embodiment the probiotic can comprise microorganisms including, but not limited to, a bacterium, a protozoan, a yeast, a fungus, a bacterial spore, a protozoal spore, a yeast spore, a fungal spore, and any combinations thereof.

The probiotic composition may be formulated such that living microorganisms are delivered to provide a benefit to the consuming animal. For example, a probiotic composition may be formulated to target delivery of at least a portion of the microorganisms to a region of the digestive system in order to promote colonization of the region by at least some of the microorganisms. Further, microorganisms may provide other benefits such as release of metabolites beneficial to the consuming animal, inhibition of pathogenic organisms, stimulation of the immune system, and inhibition of inflammatory diseases, among others.

In one embodiment, an inventive composition formulated as a probiotic includes a nutritive medium for at least some of the included microorganisms in order to support the microorganisms in a living state prior to delivery to a human or other recipient animal. In one embodiment, the probiotic comprises a nutritive medium which is a food consumed by the animal from which the microorganisms are obtained. For example, in some embodiments, the nutritive medium is a natural food found in the animal's natural wild habitat. In one embodiment the nutritive medium includes a grass, such as an organically grown and minimally processed grass.

In one embodiment, the probiotic composition comprises living or preserved microorganisms. Preserved microorganisms include, but are not limited to, dried, freeze-dried and spore forms. In one embodiment at least a portion of the microorganisms are provided as living or preserved microorganisms.

A composition may be formulated such that a unit dose of the composition contains a specified number of microorganisms. For example, a composition may contain a number of microorganisms in the range from about 1 to about 10×10¹² microorganisms per gram.

In one embodiment, the probiotic composition is a synbiotic. The synbiotic is a supplement that contains both prebiotic(s) and probiotic(s). The prebiotic(s) and the probiotic(s) work together to improve the micro flora of the intestine. In one embodiment, the synbiotic comprises at least one genetically engineered cell of the invention and at least one prebiotic. Exemplary prebiotics include, but are not limited to, fructooligosaccharides, inulin, lactulose, galactooligosaccharides, acacia gum, soyoligosaccharides, xylooligosaccharides, isomaltooligosaccharides, gentiooligosaccharides, lactosucrose, glucooligosaccharides, pecticoligosaccharides, guar gum, partially hydrolyzed guar gum, sugar alcohols, alpha glucan, beta glucan, and any combination thereof.

The probiotic compositions of the present invention comprise at least one culture of probiotic bacteria, as described above. In one embodiment, the concentration of the probiotic bacteria is from 1×10⁷ to 1×10¹¹ CFU/g of composition. In one embodiment, the concentration of the probiotic bacteria is from 1×10⁸ to 1×10¹⁸ CFU/g of composition.

The probiotic compositions of the present invention can be pharmaceutical, dietetic, nutritional or nutraceutical compositions. For example, the probiotic composition can be, but is not limited to, a medical food, a functional food, a dietary supplement, a nutritional product or a food preparation. For example, exemplary food products include, but are not limited to, beverages, yoghurts, juices, ice creams, breads, biscuits, cereals, health bars, and spreads. In one embodiment, the probiotic compositions can further comprise a buffering agent (such as e.g., sodium bicarbonate, milk, yogurt, or infant formula).

Compounds Useful within the Invention

In one aspect, the invention provides N-acyl amides. In one embodiment, the N-acyl amides modulate the activity of G protein-coupled receptors (GPCRs).

In one embodiment, the N-acyl amide is represented by Formula (1):

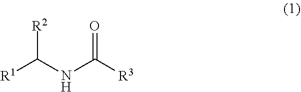

wherein:

R¹ is selected from the group consisting of carboxylate and CH₂OH;

R² is selected from the group consisting of H, (C₃-C₄)alkyl-NH₃⁺, (C₃-C₄)alkyl-NH₂, C₂ alkyl-C(═O)NH₂, CH₂OH, and methyl; and

R³ is selected from the group consisting of (C₉-C₁₈)alkyl, (C9-C₁₈)alkenyl, wherein the (C₉-C₁₈)alkyl and (C₉-C₁₈)alkenyl are optionally substituted.

In one embodiment, the N-acyl amide of Formula (1) is represented by one of Formula (2) to Formula (6):

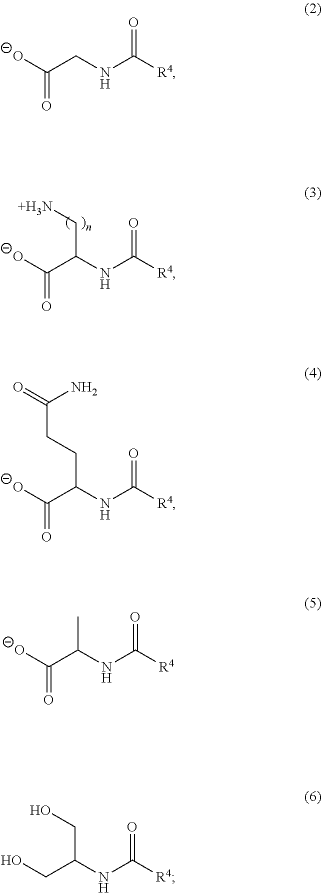

wherein:

R⁴ is selected from the group consisting of (C₉-C₁₈)alkyl, (C₉-C₁₈)alkenyl, wherein the (C₉-C₁₈)alkyl and (C₉-C₁₈)alkenyl are optionally substituted; and

n is 3 or 4.

In one embodiment, the N-acyl amide of Formula (1) is represented by one of Formulae (7)-(11):

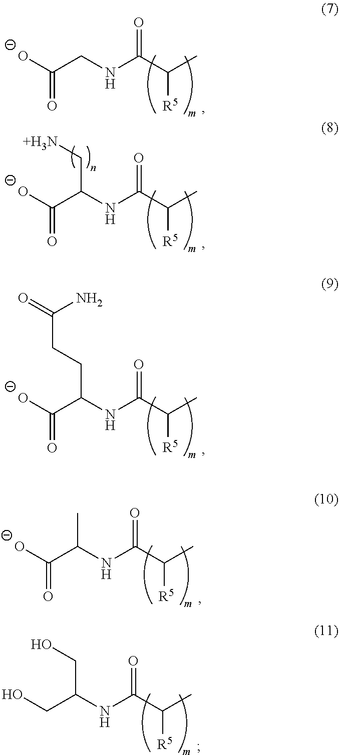

wherein:

each occurrence of R⁵ is independently selected from the group consisting of H and —OH;

and m is an integer from 8 to 17.

In one embodiment, the N-acyl amide of Formula (1) is represented by Formulae (12)-(16)

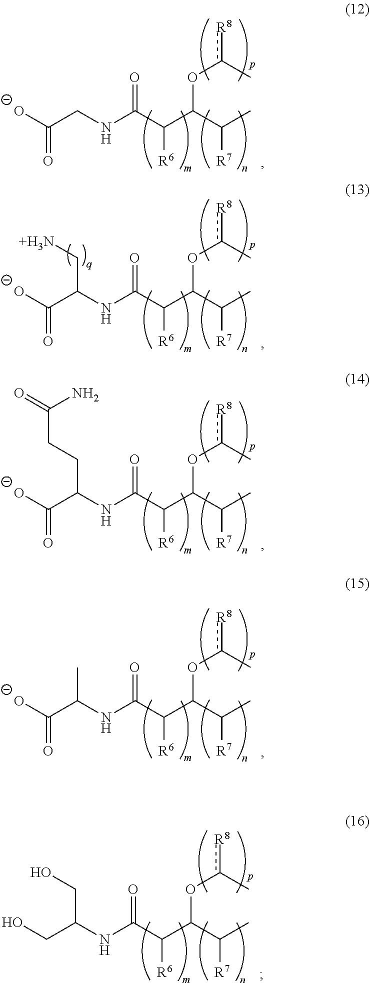

wherein:

each occurrence of R⁶, R⁷, and R⁸ is independently selected from the group consisting of H, —OH, and (═O);

m is an integer from 1 to 5;

n is an integer from 2 to 15;

p is an integer from 8 to 18; and

q is an integer from 3 to 4.

In one embodiment, the N-acyl amide is selected from the group consisting of:

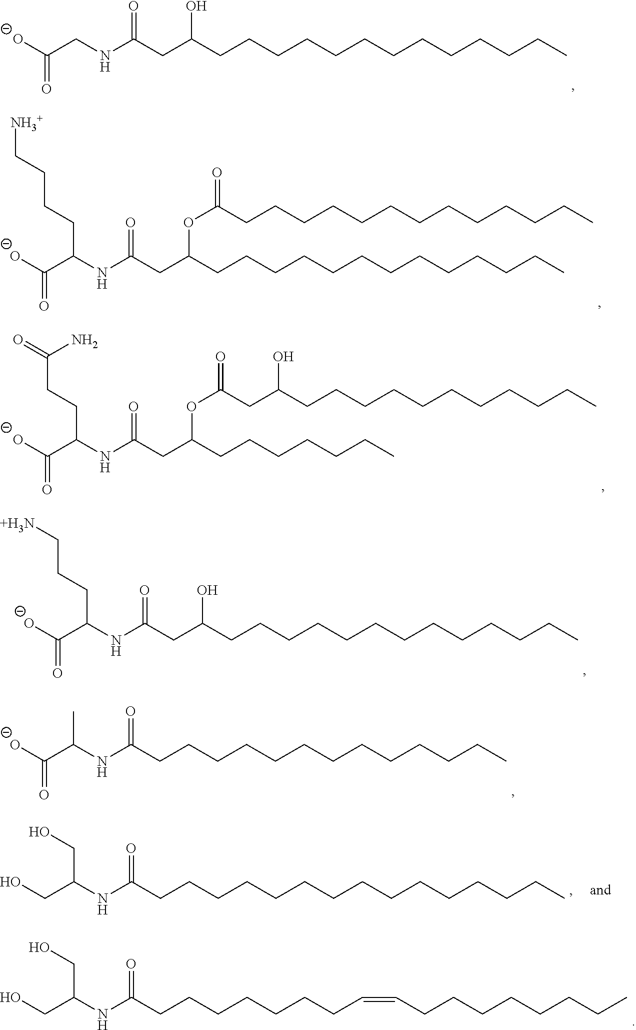

### Pharmaceutical Composition

The invention encompasses the preparation and use of pharmaceutical compositions comprising a composition of the invention. For example, in some embodiments, the of pharmaceutical composition comprises a probiotic composition, a cell expressing an hm-NAS gene, an N-acyl amide, cell expressing an N-acyl amide, an hm-NAS protein or a nucleic acid encoding an hm-NAS protein. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents.

Controlled- or sustained-release formulations of a pharmaceutical composition of the invention may be made using conventional technology.

A formulation of a pharmaceutical composition of the invention suitable for oral administration may be prepared, packaged, or sold in the form of a discrete solid dose unit including, but not limited to, a tablet, a hard or soft capsule, a cachet, a troche, or a lozenge, each containing a predetermined amount of the active ingredient. Other formulations suitable for oral administration include, but are not limited to, a powdered or granular formulation, an aqueous or oily suspension, an aqueous or oily solution, or an emulsion.

A tablet comprising the active ingredient may, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, binding agents, and lubricating agents. Known dispersing agents include, but are not limited to, potato starch and sodium starch glycollate. Known surface active agents include, but are not limited to, sodium lauryl sulphate. Known diluents include, but are not limited to, calcium carbonate, sodium carbonate, lactose, microcrystalline cellulose, calcium phosphate, calcium hydrogen phosphate, and sodium phosphate. Known granulating and disintegrating agents include, but are not limited to, corn starch and alginic acid. Known binding agents include, but are not limited to, gelatin, acacia, pre-gelatinized maize starch, polyvinylpyrrolidone, and hydroxypropyl methylcellulose. Known lubricating agents include, but are not limited to, magnesium stearate, stearic acid, silica, and talc.

Tablets may be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and U.S. Pat. No. 4,265,874 to form osmotically-controlled release tablets. Tablets may further comprise a sweetening agent, a flavoring agent, a coloring agent, a preservative, or some combination of these in order to provide pharmaceutically elegant and palatable preparation.

Hard capsules comprising the active ingredient may be made using a physiologically degradable composition, such as gelatin. Such hard capsules comprise the active ingredient, and may further comprise additional ingredients including, for example, an inert solid diluent such as calcium carbonate, calcium phosphate, or kaolin.

Soft gelatin capsules comprising the active ingredient may be made using a physiologically degradable composition, such as gelatin. Such soft capsules comprise the active ingredient, which may be mixed with water or an oil medium such as peanut oil, liquid paraffin, or olive oil.

Liquid formulations of a pharmaceutical composition of the invention which are suitable for oral administration may be prepared, packaged, and sold either in liquid form or in the form of a dry product intended for reconstitution with water or another suitable vehicle prior to use.

Liquid suspensions may be prepared using conventional methods to achieve suspension of the active ingredient in an aqueous or oily vehicle. Aqueous vehicles include, for example, water and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent.

Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, and hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e. such as with a physiologically degradable material), and methods of absorbing an aqueous or oily solution or suspension into an absorbent material, with or without subsequent drying.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, cutaneous, subcutaneous, intraperitoneal, intravenous, intramuscular, intracisternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e. powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

Formulations suitable for topical administration include, but are not limited to, liquid or semi-liquid preparations such as liniments, lotions, oil-in-water or water-in-oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent Formulations for topical administration may further comprise one or more of the additional ingredients described herein.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Pharmaceutical compositions of the invention formulated for pulmonary delivery may also provide the active ingredient in the form of droplets of a solution or suspension. Such formulations may be prepared, packaged, or sold as aqueous or dilute alcoholic solutions or suspensions, optionally sterile, comprising the active ingredient, and may conveniently be administered using any nebulization or atomization device. Such formulations may further comprise one or more additional ingredients including, but not limited to, a flavoring agent such as saccharin sodium, a volatile oil, a buffering agent, a surface active agent, or a preservative such as methylhydroxybenzoate. The droplets provided by this route of administration preferably have an average diameter in the range from about 0.1 to about 200 nanometers.

The formulations described herein as being useful for pulmonary delivery are also useful for intranasal delivery of a pharmaceutical composition of the invention.

Another formulation suitable for intranasal administration is a coarse powder comprising the active ingredient and having an average particle from about 0.2 to 500 micrometers.

Such a formulation is administered in the manner in which snuff is taken i.e. by rapid inhalation through the nasal passage from a container of the powder held close to the nares. Formulations suitable for nasal administration may, for example, comprise from about as little as 0.1% (w/w) and as much as 100% (w/w) of the active ingredient, and may further comprise one or more of the additional ingredients described herein.

A pharmaceutical composition of the invention may be prepared, packaged, or sold in a formulation suitable for buccal administration. Such formulations may, for example, be in the form of tablets or lozenges made using conventional methods, and may, for example, contain 0.1 to 20% (w/w) active ingredient, the balance comprising an orally dissolvable or degradable composition and, optionally, one or more of the additional ingredients described herein. Alternately, formulations suitable for buccal administration may comprise a powder or an aerosolized or atomized solution or suspension comprising the active ingredient. Such powdered, aerosolized, or aerosolized formulations, when dispersed, preferably have an average particle or droplet size in the range from about 0.1 to about 200 nanometers, and may further comprise one or more of the additional ingredients described herein.

A pharmaceutical composition of the invention may be prepared, packaged, or sold in a formulation suitable for ophthalmic administration. Such formulations may, for example, be in the form of eye drops including, for example, a 0.1-1.0% (w/w) solution or suspension of the active ingredient in an aqueous or oily liquid carrier. Such drops may further comprise buffering agents, salts, or one or more other of the additional ingredients described herein. Other opthalmically-administrable formulations which are useful include those which comprise the active ingredient in microcrystalline form or in a liposomal preparation.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Genaro, ed., 1985, Remington's Pharmaceutical Sciences, Mack Publishing Co., Easton, Pa., which is incorporated herein by reference.

### Methods

In one aspect, the present invention provides a method of modulating GPCR activity in a subject. In one embodiment, the method comprises administering to the subject an effective amount of a composition comprising at least one of an hm-NAS gene, an N-acyl amide, and a cell expressing an hm-NAS gene.

In one embodiment, the method comprises administering to the subject in need an effective amount of a composition that reduces the activity of one or more GPCRs. In one embodiment, the method comprises administering to the subject in need an effective amount of a composition that increases the activity of one or more GPCRs.

The GPCRs that may be modulated by the compositions and methods of the invention include, but are not limited to, ADCYAP1R1, ADORA3, ADRA1B, ADRA2A, ADRA2B, ADRA2C, ADRB1, ADRB2, AGTR1, AGTRL1, AVPR1A, AVPR1B, AVPR2, BAI1, BAI2, BAI3, BDKRB1, BDKRB2, BRS3, C3AR1, C5AR1, C5L2, CALCR, CALCRL-RAMP1, CALCRL-RAMP2, CALCRL-RAMP3, CALCR-RAMP2, CALCR-RAMP3, CCKAR, CCKBR, CCR1, CCR10, CCR2, CCR3, CCR4, CCR5, CCR6, CCR7, CCR8, CCR9, CCRL2, CHRM1, CHRM2, CHRM3, CHRM4, CHRM5, CMKLR1, CNR1, CNR2, CRHR1, CRHR2, CRTH2, CX3CR1, CXCR1, CXCR2, CXCR3, CXCR4, CXCR5, CXCR6, CXCR7, DARC, DRD1, DRD2L, DRD2S, DRD3, DRD4, DRD5, EBI2, EDG1, EDG3, EDG4, EDG5, EDG6, EDG7, EDNRA, EDNRB, F2R, F2RL1, F2RL3, FFAR1, FPR1, FPRL1, FSHR, G2A, GALR1, GALR2, GCGR, GHSR, GHSR1B, GIPR, GLP1R, GLP2R, GPR1, GPR101, GPR103, GPR107, GPR109A, GPR109B, GPR119, GPR12, GPR120, GPR123, GPR132, GPR135, GPR137, GPR139, GPR141, GPR142, GPR143, GPR146, GPR148, GPR149, GPR15, GPR150, GPR151, GPR152, GPR157, GPR161, GPR162, GPR17, GPR171, GPR173, GPR176, GPR18, GPR182, GPR20, GPR23, GPR25, GPR26, GPR27, GPR3, GPR30, GPR31, GPR32, GPR35, GPR37, GPR37L1, GPR39, GPR4, GPR45, GPR50, GPR52, GPR55, GPR6, GPR61, GPR65, GPR75, GPR78, GPR79, GPR83, GPR84, GPR85, GPR88, GPR91, GPR92, GPR97, GRPR, HCRTR1, HCRTR2, HRH1, HRH2, HRH3, HRH4, HTR1A, HTR1B, HTR1E, HTR1F, HTR2A, HTR2C, HTR5A, KISS1R, LGR4, LGR5, LGR6, LHCGR, LTB4R, MC1R, MC3R, MC4R, MC5R, MCHR1, MCHR2, MLNR, MRGPRD, MRGPRE, MRGPRF, MRGPRX1, MRGPRX2, MRGPRX4, MTNR1A, NMBR, NMU1R, NPBWR1, NPBWR2, NPFFR1, NPSR1B, NPY1R, NPY2R, NTSR1, OPN5, OPRD1, OPRK1, OPRL1, OPRM1, OXER1, OXGR1, OXTR, P2RY1, P2RY11, P2RY12, P2RY2, P2RY4, P2RY6, P2RY8, PPYR1, PRLHR, PROKR1, PROKR2, PTAFR, PTGER2, PTGER3, PTGER4, PTGFR, PTGIR, PTHR1, PTHR2, RXFP3, SCTR, SPR4, SSTR1, SSTR2, SSTR3, SSTR5, TAAR5, TACR1, TACR2, TACR3, TBXA2R, TRHR, TSHR(L), UTR2, VIPR1, and VIPR2. In one embodiment, the GPCRs that may be modulated by the compositions and methods of the invention include GPR119, SPR4, G2A, PTGIR, and PTGER4.

In one embodiment, the GPCR is enriched in the gastrointestinal mucosa. For example, in one embodiment, the method comprises administering to the subject in need an effective amount of a composition that modulates the activity of GPR119, SPR4, G2A, PTGIR, and PTGER4, or a combination thereof.

In one embodiment, the methods of the invention agonize or antagonize one or more GPCRs including, but not limited to, ADCYAP1R1, ADORA3, ADRA1B, ADRA2A, ADRA2B, ADRA2C, ADRB1, ADRB2, AGTR1, AGTRL1, AVPR1A, AVPR1B, AVPR2, BAI1, BAI2, BAI3, BDKRB1, BDKRB2, BRS3, C3AR1, C5AR1, C5L2, CALCR, CALCRL-RAMP1, CALCRL-RAMP2, CALCRL-RAMP3, CALCR-RAMP2, CALCR-RAMP3, CCKAR, CCKBR, CCR1, CCR10, CCR2, CCR3, CCR4, CCR5, CCR6, CCR7, CCR8, CCR9, CCRL2, CHRM1, CHRM2, CHRM3, CHRM4, CHRM5, CMKLR1, CNR1, CNR2, CRHR1, CRHR2, CRTH2, CX3CR1, CXCR1, CXCR2, CXCR3, CXCR4, CXCR5, CXCR6, CXCR7, DARC, DRD1, DRD2L, DRD2S, DRD3, DRD4, DRD5, EBI2, EDG1, EDG3, EDG4, EDG5, EDG6, EDG7, EDNRA, EDNRB, F2R, F2RL1, F2RL3, FFAR1, FPR1, FPRL1, FSHR, G2A, GALR1, GALR2, GCGR, GHSR, GHSR1B, GIPR, GLP1R, GLP2R, GPR1, GPR101, GPR103, GPR107, GPR109A, GPR109B, GPR119, GPR12, GPR120, GPR123, GPR132, GPR135, GPR137, GPR139, GPR141, GPR142, GPR143, GPR146, GPR148, GPR149, GPR15, GPR150, GPR151, GPR152, GPR157, GPR161, GPR162, GPR17, GPR171, GPR173, GPR176, GPR18, GPR182, GPR20, GPR23, GPR25, GPR26, GPR27, GPR3, GPR30, GPR31, GPR32, GPR35, GPR37, GPR37L1, GPR39, GPR4, GPR45, GPR50, GPR52, GPR55, GPR6, GPR61, GPR65, GPR75, GPR78, GPR79, GPR83, GPR84, GPR85, GPR88, GPR91, GPR92, GPR97, GRPR, HCRTR1, HCRTR2, HRH1, HRH2, HRH3, HRH4, HTR1A, HTR1B, HTR1E, HTR1F, HTR2A, HTR2C, HTR5A, KISS1R, LGR4, LGR5, LGR6, LHCGR, LTB4R, MC1R, MC3R, MC4R, MC5R, MCHR1, MCHR2, MLNR, MRGPRD, MRGPRE, MRGPRF, MRGPRX1, MRGPRX2, MRGPRX4, MTNR1A, NMBR, NMU1R, NPBWR1, NPBWR2, NPFFR1, NPSR1B, NPY1R, NPY2R, NTSR1, OPN5, OPRD1, OPRK1, OPRL1, OPRM1, OXER1, OXGR1, OXTR, P2RY1, P2RY11, P2RY12, P2RY2, P2RY4, P2RY6, P2RY8, PPYR1, PRLHR, PROKR1, PROKR2, PTAFR, PTGER2, PTGER3, PTGER4, PTGFR, PTGIR, PTHR1, PTHR2, RXFP3, SCTR, SPR4, SSTR1, SSTR2, SSTR3, SSTR5, TAAR5, TACR1, TACR2, TACR3, TBXA2R, TRHR, TSHR(L), UTR2, VIPR1, and VIPR2.

In one embodiment, the methods of the invention agonize or antagonize one or more GPCRs including, but not limited to, ADCYAP1R1, ADORA3, ADRA1B, ADRA2A, ADRA2B, ADRA2C, ADRB1, ADRB2, AGTR1, AGTRL1, AVPR1A, AVPR1B, AVPR2, BDKRB1, BDKRB2, BRS3, C3AR1, C5AR1, C5L2, CALCR, CALCRL-RAMP1, CALCRL-RAMP2, CALCRL-RAMP3, CALCR-RAMP2, CALCR-RAMP3, CCKAR, CCKBR, CCR10, CCR1, CCR2, CCR3, CCR4, CCR5, CCR6, CCR7, CCR8, CCR9, CHRM1, CHRM2, CHRM3, CHRM4, CHRM5, CMKLR1, CNR1, CNR2, CRHR1, CRHR2, CRTH2, CX3CR1, CXCR1, CXCR2, CXCR3, CXCR4, CXCR5, CXCR6, CXCR7, DRD1, DRD2L, DRD2S, DRD3, DRD4, DRD5, EBI2, EDG1, EDG3, EDG4, EDG5, EDG6, EDG7, EDNRA, EDNRB, F2R, F2RL1, F2RL3, FFAR1, FPR1, FPRL1, FSHR, GALR1, GALR2, GCGR, GHSR, GIPR, GLP1R, GLP2R, GPR1, GPR103, GPR109A, GPR109B, GPR119, GPR120, GPR35, GPR92, GRPR, HCRTR1, HCRTR2, HRH1, HRH2, HRH3, HRH4, HTR1A, HTR1B, HTR1E, HTR1F, HTR2A, HTR2C, HTR5A, KISS1R, LHCGR, LTB4R, MC1R, MC3R, MC4R, MC5R, MCHR1, MCHR2, MLNR, MRGPRX1, MRGPRX2, MTNR1A, NMBR, NMU1R, NPBWR1, NPBWR2, NPFFR1, NPSR1B, NPY1R, NPY2R, NTSR1, OPRD1, OPRK1, OPRL1, OPRM1, OXER1, OXTR, P2RY1, P2RY11, P2RY12, P2RY2, P2RY4, P2RY6, PPYR1, PRLHR, PROKR1, PROKR2, PTAFR, PTGER2, PTGER3, PTGER4, PTGFR, PTGIR, PTHR1, PTHR2, RXFP3, SCTR, SSTR1, SSTR2, SSTR3, SSTR5, TACR1, TACR2, TACR3, TBXA2R, TRHR, TSHR(L), UTR2, VIPR1, and VIPR2.

In one embodiment, the methods of the invention agonize one or more GPCRs including, but not limited to, BAI1, BAI2, BAI3, CCRL2, DARC, GHSR1B, GPR101, GPR107, GPR12, GPR123, GPR132, GPR135, GPR137, GPR139, GPR141, GPR142, GPR143, GPR146, GPR148, GPR149, GPR15, GPR150, GPR151, GPR152, GPR157, GPR161, GPR162, GPR17, GPR171, GPR173, GPR176, GPR18, GPR182, GPR20, GPR23, GPR25, GPR26, GPR27, GPR3, GPR30, GPR31, GPR32, GPR37, GPR37L1, GPR39, GPR4, GPR45, GPR50, GPR52, GPR55, GPR6, GPR61, GPR65, GPR75, GPR78, GPR79, GPR83, GPR84, GPR85, GPR88, GPR91, GPR97, LGR4, LGR5, LGR6, MRGPRD, MRGPRE, MRGPRF, MRGPRX4, OPN5, OXGR1, P2RY8, and TAAR5.

One of skill in the art will appreciate that the therapeutics of the invention can be administered singly or in any combination. Further, the therapeutics of the invention can be administered singly or in any combination in a temporal sense, in that they may be administered concurrently, or before, and/or after each other. One of ordinary skill in the art will appreciate, based on the disclosure provided herein, that the therapeutics compositions of the invention can be used to prevent or to treat a disease or disorder associated with abnormal GPCR activity, and that a therapeutic composition can be used alone or in any combination with another therapeutic to achieve a therapeutic result. In various embodiments, any of the therapeutics of the invention described herein can be administered alone or in combination with other therapeutics of other molecules associated a disease or disorder associated with abnormal GPCR activity.

In one embodiment, the invention provides a method of treating or preventing a disease or disorder in a subject. In one embodiment, the method comprises administering to a subject therapeutically effective amount of a composition comprising an effective amount of a composition comprising at least one of an, hm-NAS gene, an N-acyl amide, or a cell expressing an hm-NAS gene.

In one embodiment, the disease or disorder is associated with abnormal GPCR activity. For example, in some embodiments, the GPCR associated disease can include immune-related diseases, cell growth-related diseases, cell regeneration-related diseases, immunological-related cell proliferative diseases, and autoimmune diseases. Exemplary diseases and disorders associated with abnormal GPCR activity include, but are not limited to, AIDS, allergies, Alzheimer's disease, amyotrophic lateral sclerosis, atherosclerosis, bacterial, fungal, protozoan and viral infections, benign prostatic hypertrophy, bone diseases (e.g., osteoarthritis, osteoporosis), carcinoma (e.g., basal cell carcinoma, breast carcinoma, embryonal carcinoma, ovarian carcinoma, renal cell carcinoma, lung adenocarcinoma, lung small cell carcinoma, pancreatic carcinoma, prostate carcinoma, transitional carcinoma of the bladder, squamous cell carcinoma, thyroid carcinoma), cardiomyopathy, chronic and acute inflammation, circadian rhythm disorders, COPD, Crohn's disease, diabetes, Duchenne muscular dystrophy, embryonal carcinoma, endotoxic shock, environmental stress (e.g., by heat, UV or chemicals), gastrointestinal disorders, glioblastoma multiform, graft vs. host disease, Hodgkin's disease, inflammatory bowel disease, ischemia, stroke, lymphoma, macular degeneration, malignant cytokine production, malignant fibrous histiocytoma, melanoma, meningioma, mesothelioma, multiple sclerosis, gastrophoresis, autoimmune disorders, colitis, nasal congestion, pain, Parkinson's disease, prostate carcinoma, psoriasis, rhabdomyosarcoma, psychotic or neurological disorders (e.g., anxiety, depression, schizophrenia, dementia, mental retardation, memory loss, epilepsy, locomotor problems, respiratory disorders, asthma, eating/body weight disorders including obesity, bulimia, diabetes, anorexia, nausea, hypertension, hypotension), renal disorders, reperfusion injury, rheumatoid arthritis, sarcoma (e.g., chondrosarcoma, Ewing's sarcoma, osteosarcoma), septicemia, seminoma, sexual/reproductive disorders, tonsil, transitional carcinoma of the bladder, transplant rejection, trauma, tuberculosis, ulcers, ulcerative colitis, urinary retention, vascular and cardiovascular disorders, or any other disease or disorder in which G protein-coupled receptors are involved, as well as learning and/or memory disorders, diabetes, pain perception disorders, anorexia, obesity, hormonal release problems, cirrhosis, non alcoholic fatty liver disease, non alcoholic steatohepatitis, and osteopenia, or any other disease or disorder in which a specific GPCR is involved.

In one embodiment, the disease or disorder is associated with abnormal gastric emptying, appetite, or glucose homeostasis.

In one embodiment, the disease or disorder is diabetes, obesity, colitis, autoimmune disorder, atherosclerosis, gastrophoresis, cirrhosis, non-alcoholic fatty liver disease, non alcoholic steatohepatitis, or osteopenia.

It will be appreciated by one of skill in the art, when armed with the present disclosure including the methods detailed herein, that the invention is not limited to treatment of a disease or disorder associated with abnormal GPCR activity that is already established. Particularly, the disease or disorder need not have manifested to the point of detriment to the subject; indeed, the disease or disorder need not be detected in a subject before treatment is administered. That is, significant signs or symptoms of the disease or disorder do not have to occur before the present invention may provide benefit. Therefore, the present invention includes a method for preventing a disease or disorder associated with abnormal GPCR activity, in that a modulator composition, as discussed previously elsewhere herein, can be administered to a subject prior to the onset of the disease or disorder, thereby preventing the disease or disorder. The preventive methods described herein also include the treatment of a subject that is in remission for the prevention of a recurrence a disease or disorder associated with abnormal GPCR activity.

One of skill in the art, when armed with the disclosure herein, would appreciate that the prevention of a disease or disorder associated with abnormal GPCR activity, encompasses administering to a subject a modulator composition as a preventative measure against the development of, or progression of a disease or disorder associated with abnormal GPCR activity. Further, the invention encompasses treatment or prevention of such diseases discovered in the future.

Subjects to which administration of the pharmaceutical compositions of the invention is contemplated include, but are not limited to, humans and other primates, mammals including commercially relevant mammals such as non-human primates, cattle, pigs, horses, sheep, cats, and dogs. In one embodiment, the subject is a mammal. In one embodiment, the subject is a human.

The therapeutic agents may be administered under a metronomic regimen. As used herein, “metronomic” therapy refers to the administration of continuous low-doses of a therapeutic agent.

The compositions can be administered in conjunction with (e.g., before, simultaneously or following) one or more therapies. For example, in certain embodiments, the method comprises administration of a composition of the invention in conjunction with a therapeutic that alleviates the symptoms of the disease or disorder associated with a genetic mutation.

Dosage, toxicity and therapeutic efficacy of the present compositions can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. The compositions that exhibit high therapeutic indices are preferred. While compositions that exhibit toxic side effects may be used, care should be taken to design a delivery system that targets such compositions to the site of affected tissue in order to minimize potential damage to uninfected cells and, thereby, reduce side effects.

The data obtained from the cell culture assays and animal studies can be used in formulating a range of dosage for use in humans. The dosage of such compositions lies preferably within a range of circulating concentrations that include the ED50 with little or no toxicity. The dosage may vary within this range depending upon the dosage form employed and the route of administration utilized. For any composition used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of a composition (i.e., an effective dosage) means an amount sufficient to produce a therapeutically (e.g., clinically) desirable result. The compositions can be administered from one or more times per day to one or more times per week; including once every other day. The skilled artisan will appreciate that certain factors can influence the dosage and timing required to effectively treat a subject, including but not limited to the severity of the disease or disorder, previous treatments, the general health and/or age of the subject, and other diseases present. Moreover, treatment of a subject with a therapeutically effective amount of the compositions of the invention can include a single treatment or a series of treatments.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, are not to be construed as limiting in any way the remainder of the disclosure.

### Example 1: Commensal Bacteria Produce GPCR Ligands that Mimic Human Signaling Molecules

The data presented herein combines bioinformatic analysis of human microbiome sequencing data with targeted gene synthesis, heterologous expression, and high-throughput G protein-coupled receptors (GPCR) activity screening to identify GPCR-active N-acyl amides encoded by human microbiota. It is described herein that N-acyl amide biosynthetic genes are enriched in gastrointestinal bacteria and the lipids they encode interact with GPCRs that regulate gastrointestinal tract functions related to metabolism, immunity, and tissue repair. Mouse and cell-based models further demonstrated that commensal GPR119 agonists regulate metabolic hormones and glucose homeostasis as efficiently as human ligands. This work suggests that chemical mimicry of eukaryotic signaling molecules may be common among commensal bacteria and that manipulation of microbiota genes that encode metabolites capable of eliciting host cellular responses represents a new small molecule therapeutic modality (microbiome-biosynthetic-gene-therapy).

Isolation of Human Microbiota N-acyl Amides

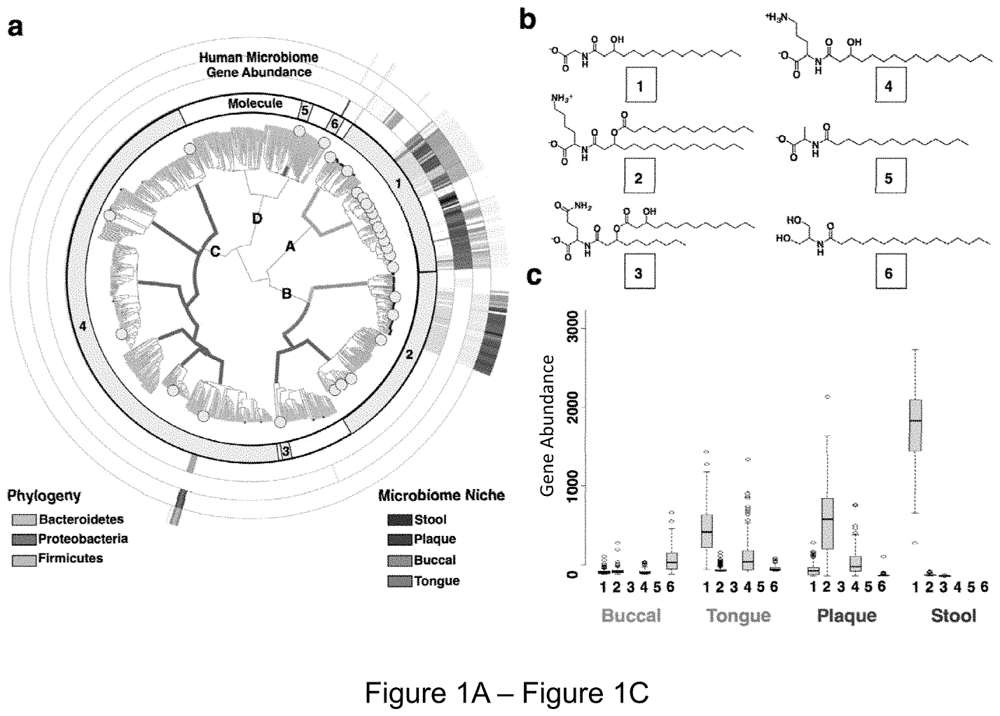

To identify NAS genes within the genomes of human microbiota, the Human Microbiome Project (HMP) sequence data was searched with BLASTN using 689 NAS genes associated with the N-acyl synthase protein family PFAM13444. The 143 unique human microbial NAS genes (hm-NASs) identified fall into four major clades (clades A-D) that are further divided into a number of distinct sub-clades (FIG. 1A). Forty-four phylogenetically diverse hm-NAS genes were selected for synthesis based on their location in the PFAM13444 phylogenetic tree and cloned into the isopropyl 3-D-1-thiogalactopyranoside (IPTG) inducible pET28c expression vector. This set included all hm-NAS genes from clades C and D, which are sparsely populated with hm-NAS sequences and multiple representative examples from clades A and B, which are heavily populated with hm-NAS sequences (FIG. 1A).

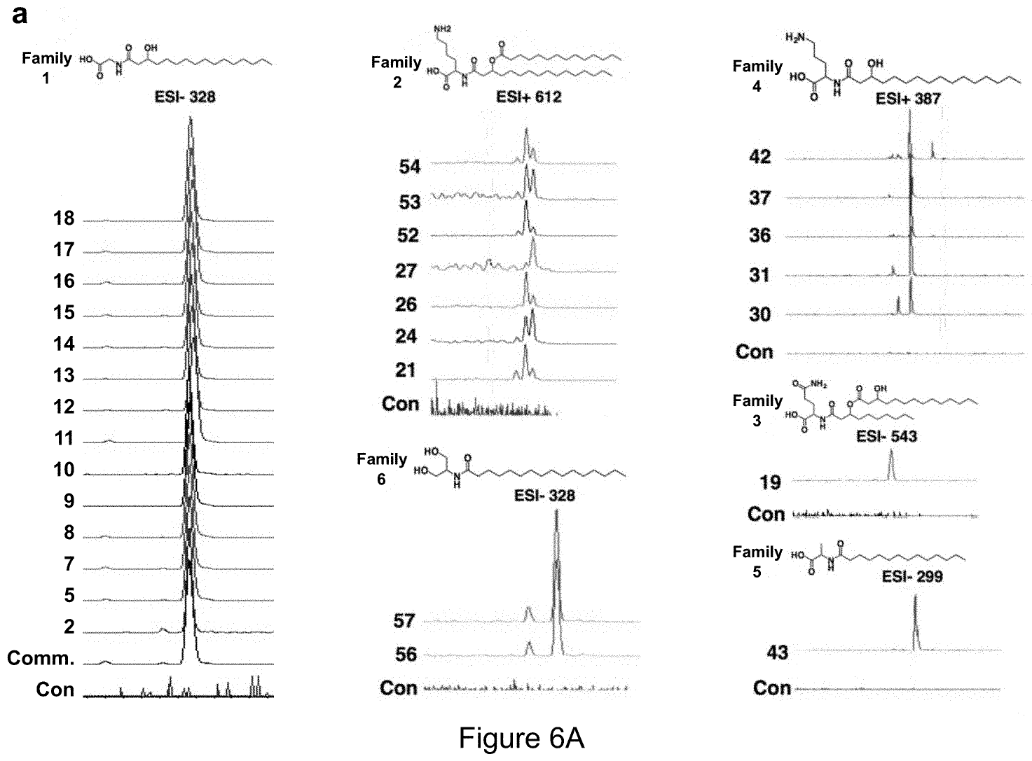

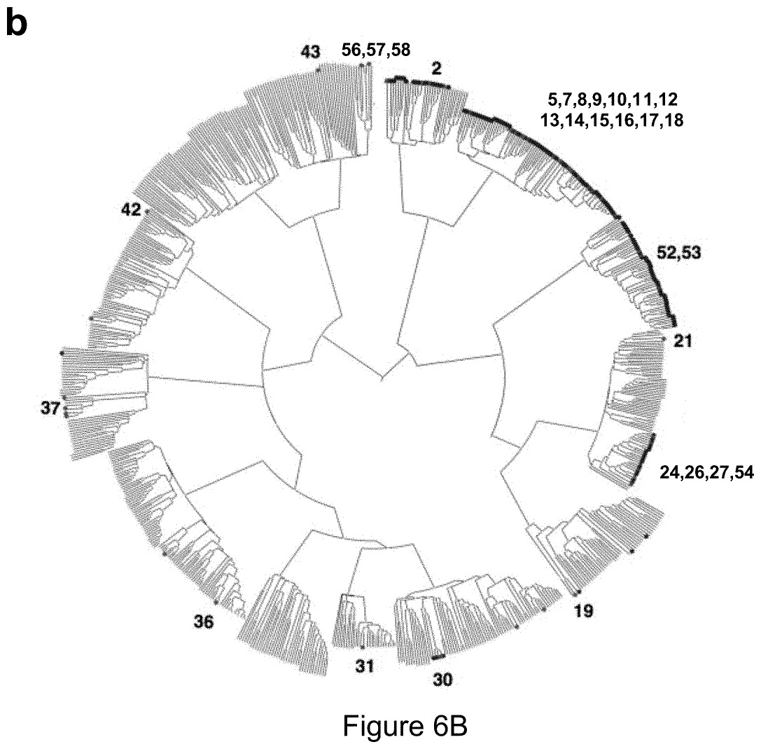

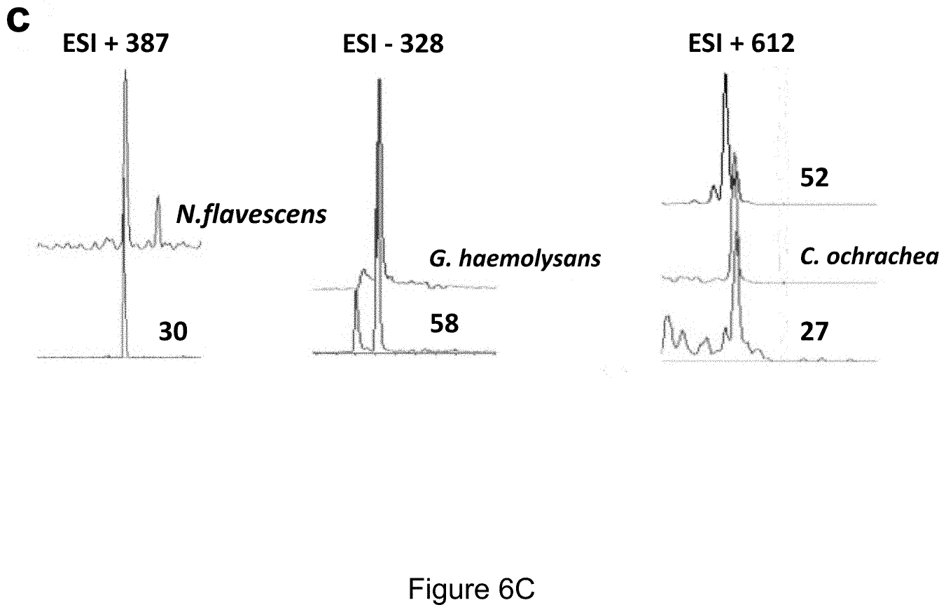

Liquid chromatography-mass spectrometry (LCMS) analysis of ethyl acetate extracts derived from IPTG induced E. coli cultures transformed with each construct revealed clone specific peaks in 30 of the 43 cultures. hm-NAS gene functions could be clustered into 6 distinct groups, based on the retention time and observed masses of the heterologously produced metabolites (FIG. 6, Table 3). Molecule isolation and structural elucidation studies were carried out for one representative culture from each group. This led to the identification of six distinct N-acyl amide families (FIG. 1B, families 1-6) that differ by both amine head group and fatty acid tail: 1)N-acyl glycine, 2)N-acyloxyacyl lysine/ornithine, 3)N-acyloxyacyl glutamine, 4)N-acyl lysine/ornithine, 5)N-acyl alanine, 6)N-acyl serinol. Each of these was isolated as a family of metabolites with slightly different fatty acid substituents. The most common analog within each family is shown in FIG. 1B. Long-chain N-acyl ornithines, lysines and glutamines have been reported as natural products produced by soil bacteria (Moore et al., 2015, Front Microbiol 6:637; Geiger et al., 2010, Prog Lipid Res 49:46-60; Zhang et al., J Am Soc Mass Spectrom 20:198-212). N-acyl ornithines are also produced by some human pathogens including Brucella abortus, Pseudomonas aeruginosa, and Burkholderia cenocepacia.

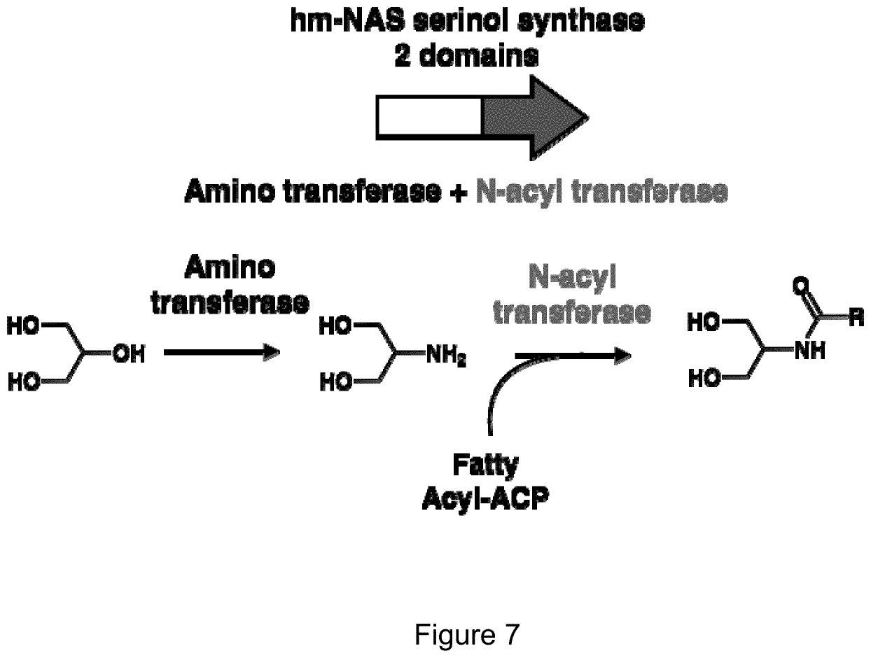

Functional differences in NAS enzymes follow the pattern of the NAS phylogenetic tree, with hm-NAS genes from the same clade or sub-clade largely encoding the same metabolite family (FIG. 1A). With the exception of the NAS that is predicted to use both lysine and ornithine as substrates, hm-NASs appear to be selective for a single amine-containing substrate such that each molecule family is comprised of an amine group linked to a range of acyl chains. The most common acyl chains incorporated by hm-NASs are from 14 to 18 carbons in length and, in some instances, are modified with either a β-hydroxylation or a single unsaturation. Three hm-NAS enzymes contain two domains. These second domains are either an aminotransferase domain that is predicted to catalyze the formation of serinol from glycerol in the biosynthesis of N-acyl serinols (FIG. 1B, family 6; FIG. 7) or an additional acyltransferase domain that is predicted to catalyze the transfer of a second acyl group to the β-hydroxyl of the N-linked acyl chain in N-acyloxyacyl glutamine/lysine/ornithine biosynthesis (FIG. 1B, families 2, 3). To explore NAS gene synteny gene occurrence patterns were identified around NAS genes in the human microbiome. The only repeating pattern observed was that some NAS genes appear adjacent to genes predicted to encode acyltransferases. This is reminiscent of the two domain NASs that produce di-acyl lipids (families 2 and 3). There were rare instances where NASs potentially occur in gene clusters, but none of these were used in this study.

To look for native N-acyl amide production by commensal bacteria, organic extracts from cultures of species containing the hm-NAS genes that were examined were screened by LCMS. Based on retention time and mass the production of the expected N-acyl amides by commensal species predicted to produce N-acyl glycines, N-acyloxyacyl lysines, N-acyl lysine/ornithines and N-acyl serinols were detected. The only case where the expected N-acyl amide was not detected was for N-acyloxyacyl glutamines (FIG. 6).

TABLE 3
hm-NAS Genes Selected for Heterologous Expression. This set
included all hm-NAS genes from clades sparsely populated with
hm-NAS sequences and representative examples from clades
heavily populated with hm-NAS sequences

| | | | Gene | |
|---|---|---|---|---|
| Clone | | | Size | Molecule |
| Number | EBI Gene | Organism | (bp) | Family |
| 1 | EFI7261 | Bacteroides oral 274 F0058 | 1191 | No |
| | | | | production |
| 2 | EHB91285 | Alistipes indistinctus YUT 12060 | 921 | 1 |
| 3 | EEK17761 | Porphyromonas uenonis | 960 | No |
| | | | | production |
| 5 | EEY82825 | Bacteroides sp 2_1_33B | 987 | 1 |
| 6 | EHP49568 | Odoribacter laneus YIT 12061 | 969 | No |
| | | | | production |
| 7 | EHG23013 | Alloprevotella rava F0323 | 1008 | 1 |
| 8 | EFA42931 | Prevotella bergensis DSM 17361 | 999 | 1 |
| 9 | EFL47029 | Prevotella disiens FB035 | 1005 | 1 |
| 10 | EHO75052 | Prevotella micans F0438 | 1005 | 1 |
| 11 | ADK95845 | Prevotella melaninogenica ATCC 25845 | 1011 | 1 |
| 12 | EFV04460 | Prevotella salivae DSM 15606 | 1017 | 1 |
| 13 | EHH01788 | Paraprevotella clara YIT 11850 | 945 | 1 |
| 14 | EDY97076 | Bacteroides plebius DSM 17135 | 1002 | 1 |
| 15 | CBW20928 | Bacteroides fragilis 638R | 1026 | 1 |
| 16 | EDS14876 | Bacteroides stercoris ATCC 43183 | 1035 | 1 |
| 17 | EDO52243 | Bacteroides uniformis ATCC 8492 | 990 | 1 |
| 18 | CBK67812 | Bacteroides xylanisolvens XB1A | 1029 | 1 |
| 19 | ACI09609 | Klebsiella pneumonia 342 | 1713 | 3 |
| 21 | ABV66681 | Acrobacter butzleri RM4018 | 1716 | 2 |
| 24 | EHT12133 | Klebsiella oxytoca 10-5246 | 1731 | 2 |
| 26 | EFE54303 | Providencia rettgeri DSM 1131 | 1743 | 2 |
| 27 | EFE94777 | Serratia odorifera DSM 4582 | 1734 | 2 |
| 29 | EER56350 | Neisseria flavescens SK114 | 768 | No |
| | | | | production |
| 30 | EET45812 | Neisseria sicca ATCC 29256 | 783 | 4 |
| 31 | ACS62992 | Ralstonia pickettii 12D | 846 | 4 |
| 33 | BAH33083 | Rhodococcus erythropolis PR4 | 849 | No |
| | | | | production |
| 35 | EFG73978 | Mycobacterium parascrofulaceum | 870 | No |
| | | ATCC BAA 614 | | production |
| 36 | CAW29482 | Pseudomonas aeruginosa LESB58 | 768 | 4 |
| 37 | EFH13337 | Roseomonas cervicalis ATCC 49957 | 813 | 4 |
| 38 | EGP09383 | Bradyrhizobiaceae bacterium SG-6C | 1041 | No |
| | | | | production |
| 39 | EEV22085 | Enhydrobacter aerosaccus SK60 | 1011 | No |
| | | | | production |
| 40 | EEY94333 | Acinetobacter junii SH205 | 789 | No |
| | | | | production |
| 41 | EFF83269 | Acinetobacter haemolyticus ATCC | 789 | No |
| | | 19194 | | production |
| 42 | CAP01857 | Acinetobacter baumannii SDF | 816 | 4 |
| 43 | EGP10046 | Bradyrhizobiaceae bacterium SG-6C | 804 | 5 |
| 50 | EFK33376 | Chryseobacterium gleum ATCC 35910 | 1854 | No |
| | | | | production |
| 51 | EEK14630 | Capnocytophaga gingivalis ATCC | 1815 | No |
| | | 33624 | | production |
| 52 | EFS97491 | Capnocytophaga ochracea F0287 | 1848 | 2 |
| 53 | CBK85930 | Enterobacter cloacae NCTC 9394 | 1713 | 2 |
| 54 | EHM48796 | Yokenella regensburgei ATCC 43003 | 1713 | 2 |
| 55 | EEK89350 | Bacilus cereus m1550 | 1596 | No |
| | | | | production |
| 56 | EHL05550 | Desulfitobacterium hafniense DP7 | 1638 | 6 |
| 57 | EFV76279 | Bacillus sp 2_A_57_CT2 | 1623 | 6 |
| 58 | GL883582 | Gemella Haemolysans M341 | 1576 | 6 |

hm-NAS Genes are Enriched in Gastrointestinal Bacteria

A BLASTN search of NAS genes against human microbial reference genomes and metagenomic sequence data from the HMP revealed that NAS genes are enriched in gastrointestinal bacteria relative to bacteria found at other body sites (Fischer's exact test p<0.05, gastrointestinal versus non-gastrointestinal sites, Table 4, FIG. 1). Within gastrointestinal sites that were frequently sampled in the context of the HMP (e.g., stool, buccal mucosa, supragingival plaque, tongue) hm-NAS gene families show distinct distribution patterns (FIG. 1C, two way ANOVA p<2e-16). Despite tremendous person-to-person variation in microbiota species composition, most N-acyl amide synthase gene families studied can be found in over 90% of patient samples. N-acyoxyacyl glutamine (12%) and N-acyl alanine (not detected) synthase genes are the only exceptions. Taken together, these data suggest that NAS genes are highly prevalent in the human microbiome and unique sites within the gastrointestinal tract are likely exposed to different sets of N-acyl amide structures.

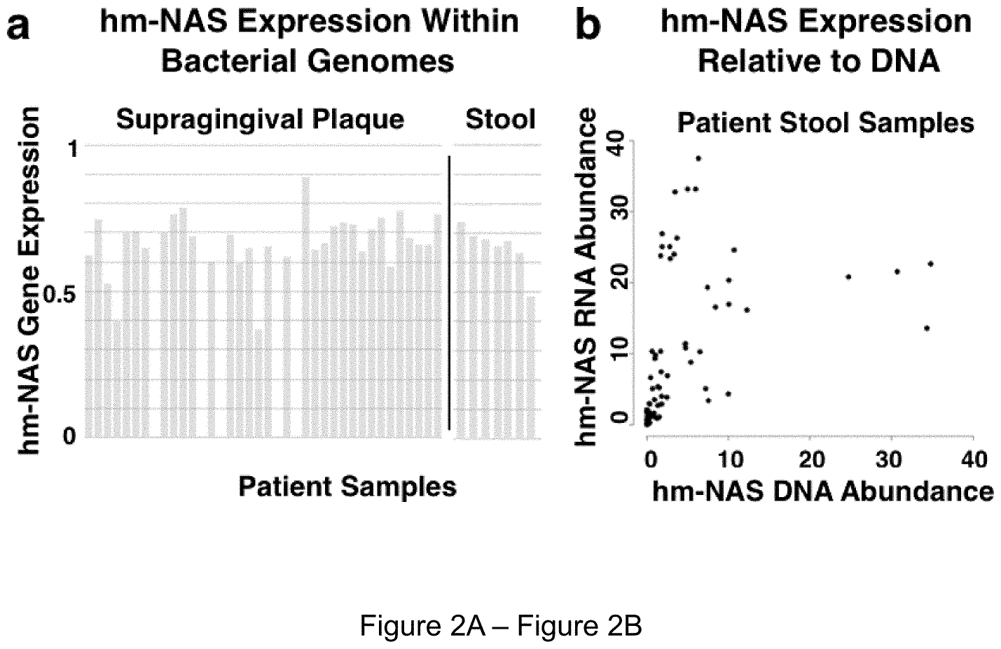

When the existing metatranscriptome sequence data from stool and supragingival plaque microbiomes was searched to look for evidence of hm-NAS gene expression in the gastrointestinal tract, site-specific hm-NAS gene expression was observed that matches the predicted body site localization patterns for hm-NAS genes in metagenomic data. Across patient samples hm-NAS genes are transcribed to varying degrees relative to the average level of transcription for each gene in the bacterial genome (FIG. 2A). In the stool metatranscriptome dataset both RNA and DNA sequencing datasets were available allowing for a more direct sample-to-sample comparison of hm-NAS gene expression levels. When metatranscriptome data were normalized using the number of hm-NAS gene specific DNA sequence reads detected in each sample, what appears to be differential expression of hm-NAS genes in different patient samples was observed (FIG. 2B). Datasets whereby bacterial genes, transcripts and metabolites can be tracked in a single sample will be necessary to explore how hm-NAS gene transcription variation impacts metabolite production.

TABLE 4
Reference Genome Analysis

| | N-acyl | | | | | | | |
|---|---|---|---|---|---|---|---|---|
| | Amide | | | | | | | |
| | Molecule | Body | | | | % | | HMP reference |
| PFAM13444 Gene | Family | site* | Phyla | Score | E-Value | Identity | Length | genome** |
| R6A3N1_9BACT/51-156 | 1 | Oral | Bacteroidetes | 110 | 2.00E−22 | 76.88 | 199 | >ADDV01000044 |
| | | | | | | | | Prevotella oris |
| | | | | | | | | C735 |
| R6EH40_9BACT/51-155 | 1 | Oral | Bacteroidetes | 281 | 3.00E−74 | 72.53 | 892 | >ADDV01000044 |
| | | | | | | | | Prevotella oris |
| | | | | | | | | C735 |
| R7PBT6_9BACT/52-156 | 1 | Oral | Bacteroidetes | 58.4 | 6.00E−07 | 100 | 31 | >ADCT01000041 |
| | | | | | | | | Prevotella sp. |
| | | | | | | | | C561 |
| R7NN97_9BACE/51-155 | 1 | GI | Bacteroidetes | 1790 | 0 | 99.59 | 981 | >AQHY01000032 |
| | | tract | | | | | | Bacteroides |
| | | | | | | | | massiliensis |
| | | | | | | | | B84634 |
| A0A0C3RD59_9PORP/51-157 | 1 | GI | Bacteroidetes | 82.4 | 4.00E−13 | 82.8 | 93 | >GG705232 |
| | | tract | | | | | | Bacteroides sp. |
| | | | | | | | | 3_1_33FAA |
| A6L081_BACV8/51-155 | 1 | GI | Bacteroidetes | 1807 | 0 | 99.9 | 981 | >ADKO01000098 |
| | | tract | | | | | | Bacteroides |
| | | | | | | | | vulgatus PC510 |
| A6LEV2_PARD8/51-155 | 1 | GI | Bacteroidetes | 1762 | 0 | 99.28 | 975 | >ACPW01000045 |
| | | tract | | | | | | Parabacteroides |
| | | | | | | | | sp. D13 |
| D4IM11_9BACT/57-158 | 1 | GI | Bacteroidetes | 1868 | 0 | 100 | 1011 | >ADKO01000098 |
| | | tract | | | | | | Bacteroides |
| | | | | | | | | vulgatus PC510 |
| D5EVS3_PRER2/52-157 | 1 | GI | Bacteroidetes | 459 | 2.00E−126 | 75.3 | 996 | >DS995534 |
| | | tract | | | | | | Bacteroides dorei |
| | | | | | | | | DSM 17855 |
| D6D060_9BACE/51-155 | 1 | GI | Bacteroidetes | 1879 | 0 | 100 | 1017 | >GG705232 |
| | | tract | | | | | | Bacteroides sp. |
| | | | | | | | | 3_1_33FAA |
| E6SVI0_BACT6/51-155 | 1 | GI | Bacteroidetes | 907 | 0 | 84.02 | 945 | >FP929032 |
| | | tract | | | | | | Alistipes shahii |
| | | | | | | | | WAL 8301 |
| CBK67812_CBK67812.1_Bacteroides_xylanisolvens_XB1A_hypothetical_protein | 1 | GI | Bacteroidetes | 1879 | 0 | 100 | 1017 | >GG703854 |
| | | tract | | | | | | Prevotella copri |
| | | | | | | | | DSM 18205 |
| ENA_CBW20928_CBW20928.1_Bacteroides_fragilis_638R_putative_hemolysin_A | 1 | GI | Bacteroidetes | 1873 | 0 | 100 | 1014 | >FP929033 |
| | | tract | | | | | | Bacteroides |
| | | | | | | | | xylanisolvens |
| | | | | | | | | XB1A |
| ENA_EDO52243_EDO52243.1_Bacteroides_uniformis_ATCC_8492_hemolysin | 1 | GI | Bacteroidetes | 1807 | 0 | 100 | 978 | >GL882689 |
| | | tract | | | | | | Bacteroides |
| | | | | | | | | fluxus YIT 12057 |
| ENA_EDS14876_EDS14876.1_Bacteroides_stercoris_ATCC_43183_hemolysin_{—} | 1 | GI | Bacteroidetes | 1890 | 0 | 100 | 1023 | >FP929033 |
| | | tract | | | | | | Bacteroides |
| | | | | | | | | xylanisolvens |
| | | | | | | | | XB1A |
| ENA_EDY97076_EDY97076.1_Bacteroides_plebeius_DSM_17135_hemolysin_{—} | 1 | GI | Bacteroidetes | 1829 | 0 | 100 | 990 | >JH636044 |
| | | tract | | | | | | Bacteroides sp. |
| | | | | | | | | 3_2_5 |
| ENA_EEY82825_EEY82825.1_Bacteroides_sp._2_l33B_hemolysin_{—} | 1 | GI | Bacteroidetes | 1801 | 0 | 100 | 975 | >ACPT01000029 |
| | | tract | | | | | | Bacteroides sp. |
| | | | | | | | | D20 |
| ENA_EFV04460_EFV04460.1_Prevotella_salivae_DSM_15606_hemolysin_{—} | 1 | GI | Bacteroidetes | 1857 | 0 | 100 | 1005 | >ABFZ02000020 |
| | | tract | | | | | | Bacteroides |
| | | | | | | | | stercoris ATCC |
| | | | | | | | | 43183 |
| ENA_EHB91285_EHB91285.1_Alistipes_indistinctus_YIT_12060_hypothetical_protein_{—} | 1 | GI | Bacteroidetes | 1679 | 0 | 100 | 909 | >ABQC02000004 |
| | | tract | | | | | | Bacteroides |
| | | | | | | | | plebeius DSM |
| | | | | | | | | 17135 |
| ENA_EHH01788_EHH01788.1_Paraprevotella_clara_YIT_11840_hemolysin | 1 | GI | Bacteroidetes | 1724 | 0 | 100 | 933 | >GG705151 |
| | | tract | | | | | | Bacteroides sp. |
| | | | | | | | | 2_1_33B |
| ENA_EHP49568_EHP49568.1_Odoribacter_laneus_YIT_12061_hypothetical_protein | 1 | GI | Bacteroidetes | 1768 | 0 | 100 | 957 | >GL629647 |
| | | tract | | | | | | Prevotella salivae |
| | | | | | | | | DSM 15606 |
| I3YLB0_ALIFI/56-157 | 1 | GI | Bacteroidetes | 941 | 0 | 84.58 | 953 | >JH370372 |
| | | tract | | | | | | Alistipes |
| | | | | | | | | indistinctus YIT |
| | | | | | | | | 12060 |
| Q5LII1_BACFN/51-155 | 1 | GI | Bacteroidetes | 1873 | 0 | 100 | 1014 | >JH376579 |
| | | tract | | | | | | Paraprevotella |
| | | | | | | | | clara YIT 11840 |
| Q8A247_BACTN/51-155 | 1 | GI | Bacteroidetes | 1873 | 0 | 100 | 1014 | >JH594596 |
| | | tract | | | | | | Odoribacter |
| | | | | | | | | laneus YIT 12061 |
| R5C642_9BACE/51-155 | 1 | GI | Bacteroidetes | 436 | 8.00E−120 | 75.43 | 924 | >FP929032 |
| | | tract | | | | | | Alistipes shahii |
| | | | | | | | | WAL 8301 |
| R5FQF1_9BACT/53-157 | 1 | GI | Bacteroidetes | 416 | 1.00E−113 | 74.59 | 972 | >ACWI01000002 |
| | | tract | | | | | | Bacteroides sp. |
| | | | | | | | | 2_1_56FAA |
| R5I942_9PORP/51-156 | 1 | GI | Bacteroidetes | 111 | 5.00E−22 | 74.23 | 291 | >JH636041 |
| | | tract | | | | | | Bacteroides sp. |
| | | | | | | | | 1_1_6 |
| R5JGR8_9BACE/51-155 | 1 | GI | Bacteroidetes | 1823 | 0 | 99.6 | 999 | >KB905466 |
| | | tract | | | | | | Bacteroides |
| | | | | | | | | salyersiae WAL |
| | | | | | | | | 10018 |
| R5KD71_9BACT/52-157 | 1 | GI | Bacteroidetes | 606 | 6.00E−171 | 78.43 | 955 | >GL629647 |
| | | tract | | | | | | Prevotella salivae |
| | | | | | | | | DSM 15606 |
| R5MMX8_9BACE/51-155 | 1 | GI | Bacteroidetes | 1768 | 0 | 98.99 | 987 | >ACWH01000030 |
| | | tract | | | | | | Bacteroides |
| | | | | | | | | ovatus |
| | | | | | | | | 3_8_47FAA |
| R5NZI1_9BACT/51-155 | 1 | GI | Bacteroidetes | 1690 | 0 | 99.36 | 933 | >KB905466 |
| | | tract | | | | | | Bacteroides |
| | | | | | | | | salyersiae WAL |
| | | | | | | | | 10018 |
| R5UEV5_9BACE/51-155 | 1 | GI | Bacteroidetes | 1857 | 0 | 99.7 | 1014 | >JH379426 |
| | | tract | | | | | | Prevotella |
| | | | | | | | | stercorea DSM |
| | | | | | | | | 18206 |
| R5UPI5_9PORP/51-157 | 1 | GI | Bacteroidetes | 1762 | 0 | 99.9 | 957 | >ABJL02000006 |
| | | tract | | | | | | Bacteroides |
| | | | | | | | | intestinalis DSM |
| | | | | | | | | 17393 |
| R5VW07_9BACE/51-155 | 1 | GI | Bacteroidetes | 1546 | 0 | 94.85 | 990 | >JH376579 |
| | | tract | | | | | | Paraprevotella |
| | | | | | | | | clara YIT 11840 |
| R6B4U0_9BACT/52-156 | 1 | GI | Bacteroidetes | 726 | 0 | 80.02 | 991 | >AAVM0200000 |
| | | tract | | | | | | 9 Bacteroides |
| | | | | | | | | caccae ATCC |
| | | | | | | | | 43185 |
| R6BXV9_9BACT/52-157 | 1 | GI | Bacteroidetes | 1707 | 0 | 97.87 | 987 | >GG703854 |
| | | tract | | | | | | Prevotella copri |
| | | | | | | | | DSM 18205 |
| R6DH15_9BACE/51-155 | 1 | GI | Bacteroidetes | 1120 | 0 | 86.61 | 1016 | >GG688329 |
| | | tract | | | | | | Bacteroides |
| | | | | | | | | finegoldii DSM |
| | | | | | | | | 17565 |
| R6FKP1_9BACE/51-155 | 1 | GI | Bacteroidetes | 789 | 0 | 81.18 | 983 | >DS499674 |
| | | tract | | | | | | Bacteroides |
| | | | | | | | | stercoris ATCC |
| | | | | | | | | 43183 |
| R6FUQ8_9BACT/52-158 | 1 | GI | Bacteroidetes | 1474 | 0 | 93.45 | 993 | >JH379426 |
| | | tract | | | | | | Prevotella |
| | | | | | | | | stercorea DSM |
| | | | | | | | | 18206 |
| R6KTM3_9BACE/51-155 | 1 | GI | Bacteroidetes | 1807 | 0 | 99.7 | 987 | >ACCH01000127 |
| | | tract | | | | | | Bacteroides |
| | | | | | | | | cellulosilyticus |
| | | | | | | | | DSM 14838 |
| R6LNJ9_9BACE/51-154 | 1 | GI | Bacteroidetes | 1812 | 0 | 99.8 | 987 | >AFBM01000001 |
| | | tract | | | | | | Bacteroides clarus |
| | | | | | | | | YIT 12056 |
| R6MX16_9BACE/51-155 | 1 | GI | Bacteroidetes | 817 | 0 | 81.75 | 981 | >DS981492 |
| | | tract | | | | | | Bacteroides |
| | | | | | | | | coprocola DSM |
| | | | | | | | | 17136 |
| R6QE29_9BACT/52-157 | 1 | GI | Bacteroidetes | 785 | 0 | 81.74 | 942 | >GG703854 |
| | | tract | | | | | | Prevotella copri |
| | | | | | | | | DSM 18205 |
| R6S950_9BACE/51-155 | 1 | GI | Bacteroidetes | 1862 | 0 | 99.8 | 1014 | >GG688329 |
| | | tract | | | | | | Bacteroides |
| | | | | | | | | finegoldii DSM |
| | | | | | | | | 17565 |
| R6SC61_9BACE/51-155 | 1 | GI | Bacteroidetes | 1807 | 0 | 99.8 | 984 | >ACB W01000097 |
| | | tract | | | | | | Bacteroides |
| | | | | | | | | coprophilus DSM |
| | | | | | | | | 18228 |
| R6VUA1_9BACT/56-157 | 1 | GI | Bacteroidetes | 970 | 0 | 85.5 | 931 | >FP929032 |
| | | tract | | | | | | Alistipes shahii |
| | | | | | | | | WAL 8301 |
| R6XGV7_9BACT/52-157 | 1 | GI | Bacteroidetes | 390 | 6.00E−106 | 74.54 | 923 | >GG703854 |
| | | tract | | | | | | Prevotella copri |
| | | | | | | | | DSM 18205 |
| R6YIB5_9BACE/51-155 | 1 | GI | Bacteroidetes | 442 | 2.00E−121 | 76.02 | 880 | >ACTC01000036 |
| | | tract | | | | | | Bacteroides sp. |
| | | | | | | | | 4_1_36 |
| R7DDR3_9P0RP/51-155 | 1 | GI | Bacteroidetes | 1657 | 0 | 98.31 | 945 | >ACWX01000035 |
| | | tract | | | | | | Tannerella sp. |
| | | | | | | | | 6_1_58FAA CT1 |
| R7EIP8_9BACE/51-155 | 1 | GI | Bacteroidetes | 1768 | 0 | 99.28 | 978 | >ACPT01000029 |
| | | tract | | | | | | Bacteroides sp. |
| | | | | | | | | D20 |
| R7F021_9BACT/51-157 | 1 | GI | Bacteroidetes | 76.8 | 2.00E−11 | 90 | 60 | >AFZZ01000132 |
| | | tract | | | | | | Prevotella |
| | | | | | | | | stercorea DSM |
| | | | | | | | | 18206 |
| R7HSG0_9BACT/37-143 | 1 | GI | Bacteroidetes | 126 | 2.00E−26 | 72.5 | 440 | >AFZZ01000132 |
| | | tract | | | | | | Prevotella |
| | | | | | | | | stercorea DSM |
| | | | | | | | | 18206 |
| R7IYP9_9BACT/59-165 | 1 | GI | Bacteroidetes | 233 | 1.00E−58 | 72.63 | 844 | >JH379426 |
| | | tract | | | | | | Prevotella |
| | | | | | | | | stercorea DSM |
| | | | | | | | | 18206 |
| R7JHM4_9BACT/51-152 | 1 | GI | Bacteroidetes | 1829 | 0 | 99.9 | 993 | >ABFK02000017 |
| | | tract | | | | | | Alistipes |
| | | | | | | | | putredinis DSM |
| | | | | | | | | 17216 |
| E6K481_9BACT/52-156 | 1 | Oral | Bacteroidetes | 1834 | 0 | 100 | 993 | >AEPD01000010 |
| | | | | | | | | Prevotella buccae |
| | | | | | | | | ATCC 33574 |
| ENA_ADK95845_ADK95845.1_Prevotella_melaninogenica_ATCC_25845_hemolysin_{—} | 1 | Oral | Bacteroidetes | 1845 | 0 | 100 | 999 | >CP002122 |
| | | | | | | | | Prevotella |
| | | | | | | | | melaninogenica |
| | | | | | | | | ATCC 25845 |
| ENA_EFI17261_EFI17261.1_Bacteroidetes_oral_taxon_274_str._F0058_hemolysin | 1 | Oral | Bacteroidetes | 2176 | 0 | 100 | 1178 | >ADCM01000011 |
| | | | | | | | | Bacteroidetes |
| | | | | | | | | oral taxon 274 str. |
| | | | | | | | | F0058 |
| ENA_EHG23013_EHG23013.1_Alloprevotella_rava_F0323_hypothetical_protein | 1 | Oral | Bacteroidetes | 1840 | 0 | 100 | 996 | >JH376829 |
| | | | | | | | | Prevotella sp. oral |
| | | | | | | | | taxon 302 str. |
| | | | | | | | | F0323 |
| ENA_EHO75052_EHO75052.1_Prevotella_micans_F0438_hypothetical_protein | 1 | Oral | Bacteroidetes | 1834 | 0 | 100 | 993 | >JH594521 |
| | | | | | | | | Prevotella micans |
| | | | | | | | | F0438 |
| F2KX19_PREDF/64-168 | 1 | Oral | Bacteroidetes | 1895 | 0 | 100 | 1026 | >CP002589 |
| | | | | | | | | Prevotella |
| | | | | | | | | denticola F0289 |
| F9D3S1_PREDD/52-156_1 | 1 | Oral | Bacteroidetes | 1879 | 0 | 100 | 1017 | >GL982488 |
| | | | | | | | | Prevotella dentalis |
| | | | | | | | | DSM 3688 |
| I1YUM9_PREI7/53-157 | 1 | Oral | Bacteroidetes | 364 | 1.00E−98 | 73.71 | 985 | >GG703886 |
| | | | | | | | | Prevotella oris |
| | | | | | | | | F0302 |
| Q7MTR9_PORGI/53-158 | 1 | Oral | Bacteroidetes | 1801 | 0 | 100 | 975 | >AJZS01000078 |
| | | | | | | | | Porphyromonas |
| | | | | | | | | gingivalis W50 |
| R5CSR0_9BACT/52-157 | 1 | Oral | Bacteroidetes | 420 | 3.00E−115 | 75.28 | 906 | >AWEY01000007 |
| | | | | | | | | Prevotella |
| | | | | | | | | baroniae F0067 |
| R5GFN8_9BACT/51-155 | 1 | Oral | Bacteroidetes | 134 | 4.00E−29 | 70.21 | 866 | >ACZS01000081 |
| | | | | | | | | Prevotella sp. oral |
| | | | | | | | | taxon 472 str. |
| | | | | | | | | F0295 |
| R5Q4D6_9BACT/52-157 | 1 | Oral | Bacteroidetes | 392 | 6.00E−107 | 74.28 | 972 | >AWET01000051 |
| | | | | | | | | Prevotella |
| | | | | | | | | pleuritidis F0068 |
| R6W2Q2_9BACT/52-156 | 1 | Oral | Bacteroidetes | 569 | 3.00E−160 | 77.34 | 993 | >GL872283 |
| | | | | | | | | Prevotella |
| | | | | | | | | multiformis DSM |
| | | | | | | | | 16608 |
| R7CYB8_9BACE/51-155 | 1 | Oral | Bacteroidetes | 87.9 | 3.00E−15 | 71.47 | 375 | >CP002122 |
| | | | | | | | | Prevotella |
| | | | | | | | | melaninogenica |
| | | | | | | | | ATCC 25845 |
| W0EP20_9PORP/51-155 | 1 | Oral | Bacteroidetes | 180 | 5.00E−43 | 71.8 | 773 | >AWEY01000007 |
| | | | | | | | | Prevotella |
| | | | | | | | | baroniae F0067 |
| C7M608_CAPOD/352-453 | 2 | Oral | Bacteroidetes | 3230 | 0 | 98.42 | 1836 | >AMEV01000023 |
| | | | | | | | | Capnocytophaga |
| | | | | | | | | sp. oral taxon 324 |
| | | | | | | | | str. F0483 |
| ENA_EEK14630_EEK14630.1_Capnocytophaga_gingivalis_ATCC_3_3624_Acyltransferase_{—} | 2 | Oral | Bacteroidetes | 3330 | 0 | 100 | 1803 | >ACLQ01000018 |
| | | | | | | | | Capnocytophaga |
| | | | | | | | | gingivalis ATCC |
| | | | | | | | | 33624 |
| ENA_EFS97491_EFS97491.1_Capnocytophaga_ochracea_F0287_Acyltransferase | 2 | Oral | Bacteroidetes | 3391 | 0 | 100 | 1836 | >AKFV01000035 |
| | | | | | | | | Capnocytophaga |
| | | | | | | | | ochracea str. Holt |
| | | | | | | | | 25 |
| F9YU78_CAPCC/3_51-452 | 2 | Oral | Bacteroidetes | 612 | 8.00E−173 | 73.1 | 1792 | >AMEV01000023 |
| | | | | | | | | Capnocytophaga |
| | | | | | | | | sp. oral taxon 324 |
| | | | | | | | | str. F0483 |
| H1Z9S5_MYROD/346-447 | 2 | Oral | Bacteroidetes | 172 | 2.00E−40 | 72.59 | 540 | >ALNN01000028 |
| | | | | | | | | Capnocytophaga |
| | | | | | | | | sp. CM59 |
| ENA_EFA42931_EFA42931.1_Prevotella_bergensis_DSM_17361_hemolysin | 1 | Oral | Bacteroidetes | 1823 | 0 | 100 | 987 | >GG704783 |
| | | | | | | | | Prevotella |
| | | | | | | | | bergensis DSM |
| | | | | | | | | 17361 |
| A0A095ZG93_9BACT/52-156 | 1 | Oral | Bacteroidetes | 1596 | 0 | 95.41 | 1002 | >ADEG01000046 |
| | | | | | | | | Prevotella |
| | | | | | | | | buccalis ATCC |
| | | | | | | | | 35310 |
| E7RNE3_9BACT/52-156 | 1 | Oral | Bacteroidetes | 1829 | 0 | 100 | 990 | >AEPE02000002 |
| | | | | | | | | Prevotella oralis |
| | | | | | | | | ATCC 33269 |
| ENA_EEK17761_EEK17761.1_Porphyromonas_uenonis_60-3_hemolysin_{—} | 1 | Oral | Bacteroidetes | 1751 | 0 | 100 | 948 | >ACLR01000009 |
| | | | | | | | | Porphyromonas |
| | | | | | | | | uenonis 60-3 |
| ENA_EFL47029_EFL47029.1_Prevotella_disiens_FB035-09AN_hemolysin_{—} | 1 | Oral | Bacteroidetes | 1834 | 0 | 100 | 993 | >AEDO01000009 |
| | | | | | | | | Prevotella disiens |
| | | | | | | | | FB035-09AN |
| F4KL89_PORAD/55-160 | 1 | Oral | Bacteroidetes | 1735 | 0 | 99.48 | 954 | >AENO01000054 |
| | | | | | | | | Porphyromonas |
| | | | | | | | | asaccharolytica |
| | | | | | | | | PR426713P-I |
| I4Z8L9_9BACT/52-156 | 1 | Oral | Bacteroidetes | 1829 | 0 | 100 | 990 | >ADFO01000053 |
| | | | | | | | | Prevotella bivia |
| | | | | | | | | JCVTHMP010 |
| R6CE12_9BACE/51-155 | 1 | Oral | Bacteroidetes | 75 | 1.00E−11 | 72.32 | 289 | >AEDO01000009 |
| | | | | | | | | Prevotella disiens |
| | | | | | | | | FB035-09AN |
| R6XAK6_9BACT/52-156 | 1 | Oad | Bacteroidetes | 436 | 1.00E−120 | 75.76 | 887 | >AEPE02000002 |
| | | | | | | | | Prevotella oralis |
| | | | | | | | | ATCC 33269 |
| ENA_EHL05550_EHL05550.1_Desulfitobacterium_hafniense_DP7_aminotransferase_class_V_{—} | 6 | GI | Firmicutes | 3003 | 0 | 100 | 1626 | >JH414482 |
| | | tract | | | | | | Desulfitobacterium |
| | | | | | | | | hafniense DP7 |
| ENA_EFV76279_EFV76279.1_Bacillus_sp._2_A_57_CT2_serine-pyruvate_aminotransferase | 6 | Oral | Firmicutes | 2976 | 0 | 100 | 1611 | >GL635754 |
| | | | | | | | | Bacillus sp. |
| | | | | | | | | 2_A_57_CT2 |
| A6T596_KLEP7/322-423 | 2 | Oral | Proteobacteria | 3081 | 0 | 99.01 | 1719 | >JH930419 |
| | | | | | | | | Klebsiella |
| | | | | | | | | pneumoniae |
| | | | | | | | | subsp. |
| | | | | | | | | pneumoniae |
| | | | | | | | | WGLW2 |
| D8MWX6_ERWBE/367-468 | 2 | Oral | Proteobacteria | 525 | 3.00E−147 | 73.37 | 1506 | >GG753567 |
| | | | | | | | | Serratia odorifera |
| | | | | | | | | DSM 4582 |
| ENA_EFE94777_EFE94777.1_Serratia_odorifera_DSM_4582_Acyltransferase | 2 | Oral | Proteobacteria | 3181 | 0 | 100 | 1722 | >GG753567 |
| | | | | | | | | Serratia odorifera |
| | | | | | | | | DSM 4582 |
| Q6CZN2_PECAS/322-423 | 2 | | Proteobacteria | 399 | 2.00E−109 | 71.6 | 1634 | >ADBY01000051 |
| | | | | | | | | Serratia odorifera |
| | | | | | | | | DSM 4582 |
| A0A0B5CH45_NEIEG/32-132 | 4 | Omi | Proteobacteria | 1386 | 0 | 100 | 750 | >ADBF01000232 |
| | | | | | | | | Neisseria elongata |
| | | | | | | | | subsp. glycolytica |
| | | | | | | | | ATCC 29315 |
| E5UJR0_NEIMU/32-132 | 4 | Oral | Proteobacteria | 1397 | 0 | 100 | 756 | >ACRG01000005 |
| | | | | | | | | Neisseria mucosa |
| | | | | | | | | C102 |
| ENA_EET45812_EET45812.1_Neisseria_siccaATCC_29256_hypothetical_protein | 4 | Chat | Proteobacteria | 1424 | 0 | 100 | 771 | >ACK002000002 |
| | | | | | | | | Neisseria sicca |
| | | | | | | | | ATCC 29256 |
| ENA_ACI09609_ACI09609.1_Klebsiella_pneumoniae_342_conserved_hypothetical_protein | 3 | GI | Proteobacteria | 3059 | 0 | 99.12 | 1701 | >ACXA01000063 |
| | | tract | | | | | | Klebsiella sp. |
| | | | | | | | | 1155 |
| A4W746_ENT38/322-423 | 2 | GI | Proteobacteria | 1417 | 0 | 81.88 | 1689 | >FP929040 |
| | | tract | | | | | | Enterobacter |
| | | | | | | | | cloacae subsp. |
| | | | | | | | | cloacae NCTC |
| | | | | | | | | 9394 |
| ENA_CBK85930_CBK85930.1_Enterobacter_cloacae_subsp._cloacae_NCTC_9394_Putative_hemolysin_{—} | 2 | GI | Proteobacteria | 3142 | 0 | 100 | 1701 | >FP929040 |
| | | tract | | | | | | Enterobacter |
| | | | | | | | | cloacae subsp. |
| | | | | | | | | cloacae NCTC |
| | | | | | | | | 9394 |
| ENA_EFE54303_EFE54303.1_Providencia_rettgeri_DSM_1131_Acyltransferase | 2 | GI | Proteobacteria | 3197 | 0 | 100 | 1731 | >ACCI02000039 |
| | | tract | | | | | | Providencia |
| | | | | | | | | rettgeri DSM |
| | | | | | | | | 1131 |
| ENA_EHM48796_EHM48796.1_Yokenella_regensburgei_ATCC_43003_Acyltransferase | 2 | GI | Proteobacteria | 3142 | 0 | 100 | 1701 | >JH417874 |
| | | tract | | | | | | Yokenella |
| | | | | | | | | regensburgei |
| | | | | | | | | ATCC 43003 |
| F9ZAJ4_ODOSD/341-443 | 2 | GI | Proteobacteria | 1013 | 0 | 77.5 | 1738 | >JH594597 |
| | | tract | | | | | | Odoribacter |
| | | | | | | | | laneus YIT 12061 |
| G9Z3T1_9ENTR/322-423 | 2 | GI | Proteobacteria | 3142 | 0 | 100 | 1701 | >JH417874 |
| | | tract | | | | | | Yokenella |
| | | | | | | | | regensburgei |
| | | | | | | | | ATCC 43003 |
| R5UYM1_9PORP/338-439 | 2 | GI | Proteobacteria | 3314 | 0 | 99.89 | 1800 | >ADMC01000028 |
| | | tract | | | | | | Odoribacter |
| | | | | | | | | laneus YIT 12061 |
| ENA_ACS62992_ACS62992.1_Ralstonia_pickettii_12D_conserved_hypothetical_protein_{—} | 4 | GI | Proteobacteria | 1541 | 0 | 100 | 834 | >GL520222 |
| | | tract | | | | | | Ralstonia sp. |
| | | | | | | | | 5_7_47FAA |
| ENA_CAW29482_CAW29482.1_Pseudomonas_aeruginosa_LESB58_putative_hemolysin_{—} | 4 | GI | Proteobacteria | 1369 | 0 | 99.34 | 756 | >ACWU01000206 |
| | | tract | | | | | | Pseudomonas |
| | | | | | | | | sp. 2126 |
| A0A089UDH2_9ENTR/323-424 | 2 | Oral | Proteobacteria | 870 | 0 | 76.22 | 1695 | >ALNJ01000086 |
| | | | | | | | | Klebsiella sp. |
| | | | | | | | | OBRC7 |
| E6WAC8_PANSA/322-423 | 2 | Oral | Proteobacteria | 233 | 7.00E−59 | 72.78 | 709 | >GL892086 |
| | | | | | | | | Enterobacter |
| | | | | | | | | hormaechei |
| | | | | | | | | ATCC 49162 |
| ENA_EHT12133_EHT12133.1_Raoultella_omithinolytica_10-5246_hypothetical_protein | 2 | Oral | Proteobacteria | 1829 | 0 | 85.9 | 1723 | >ALNJ01000086 |
| | | | | | | | | Klebsiella sp. |
| | | | | | | | | OBRC7 |
| G7LV45_9ENTR/322-423 | 2 | Oral | Proteobacteria | 387 | 5.00E−105 | 75.31 | 875 | >ALNJ01000086 |
| | | | | | | | | Klebsiella sp. |
| | | | | | | | | OBRC7 |
| ENA_EER56350_EER56350.1_Neisseria_flavescens_SK114_hypothetical_protein_{—} | 4 | Oral | Proteobacteria | 1397 | 0 | 100 | 756 | >ACQV01000022 |
| | | | | | | | | Neisseria |
| | | | | | | | | flavescens SKI 14 |
| A0A077KL19_9FLAO/353-454 | 2 | Oral | Proteobacteria | 2289 | 0 | 89.09 | 1842 | >GL379781 |
| | | | | | | | | Chryseobacterium |
| | | | | | | | | gleum ATCC |
| | | | | | | | | 35910 |
| A7MLT3_CROS8/322-423 | 2 | tract | Proteobacteria | 630 | 1.00E−178 | 74.42 | 1591 | >AMLL01000012 |
| | | | | | | | | Klebsiella |
| | | | | | | | | pneumoniae |
| | | | | | | | | subsp. |
| | | | | | | | | pneumoniae |
| | | | | | | | | WGLW1 |
| ENA_EFK33376_EFK33376.1_Chryseobacterium_gleum_ATCC_35910_Acyltransferase_{—} | 2 | Oral | Proteobacteria | 3402 | 0 | 100 | 1842 | >GL379781 |
| | | | | | | | | Chryseobacterium |
| | | | | | | | | gleum ATCC |
| | | | | | | | | 35910 |
| ENA_CAPO_1857_CAP01857.2_Acinetobacter_baumannii_SDF_conserved_hypothetical_protein_{—} | 4 | Pathogen | Proteobacteria | 1441 | 0 | 99 | 804 | >ACQB01000026 |
| | | | | | | | | Acinetobacter |
| | | | | | | | | baumannii ATCC |
| | | | | | | | | 19606 |

hm-N-acyl-Amides Interact with GI Mucosal GPCRs

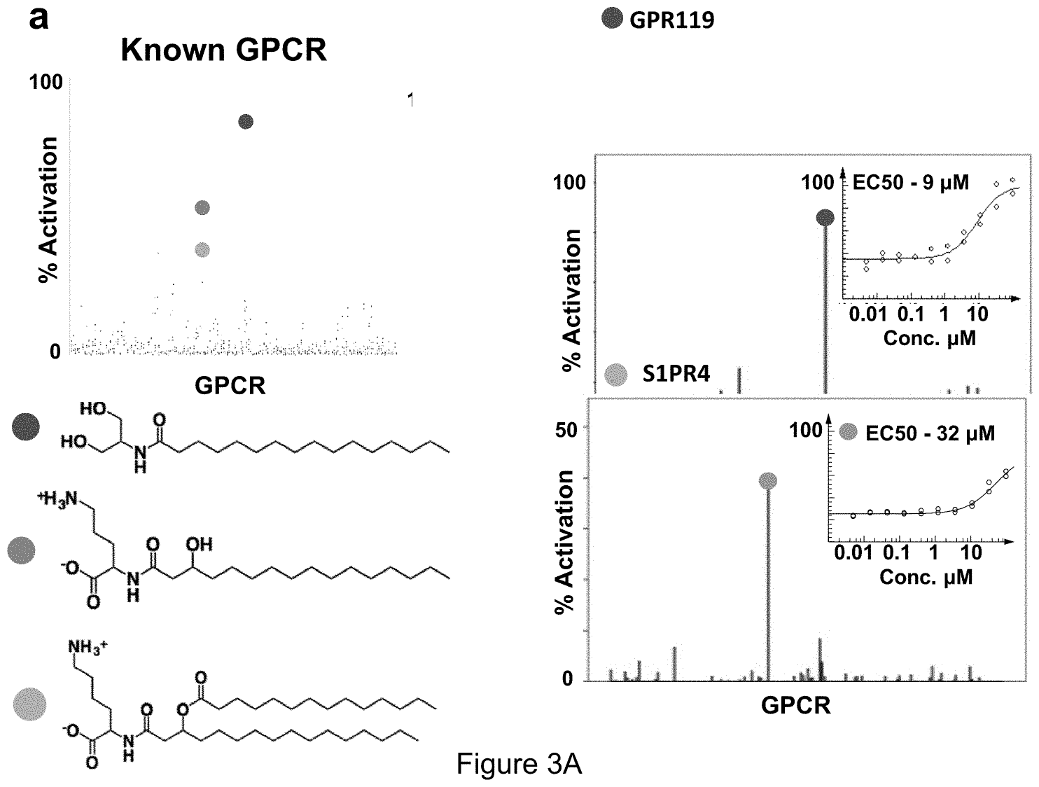

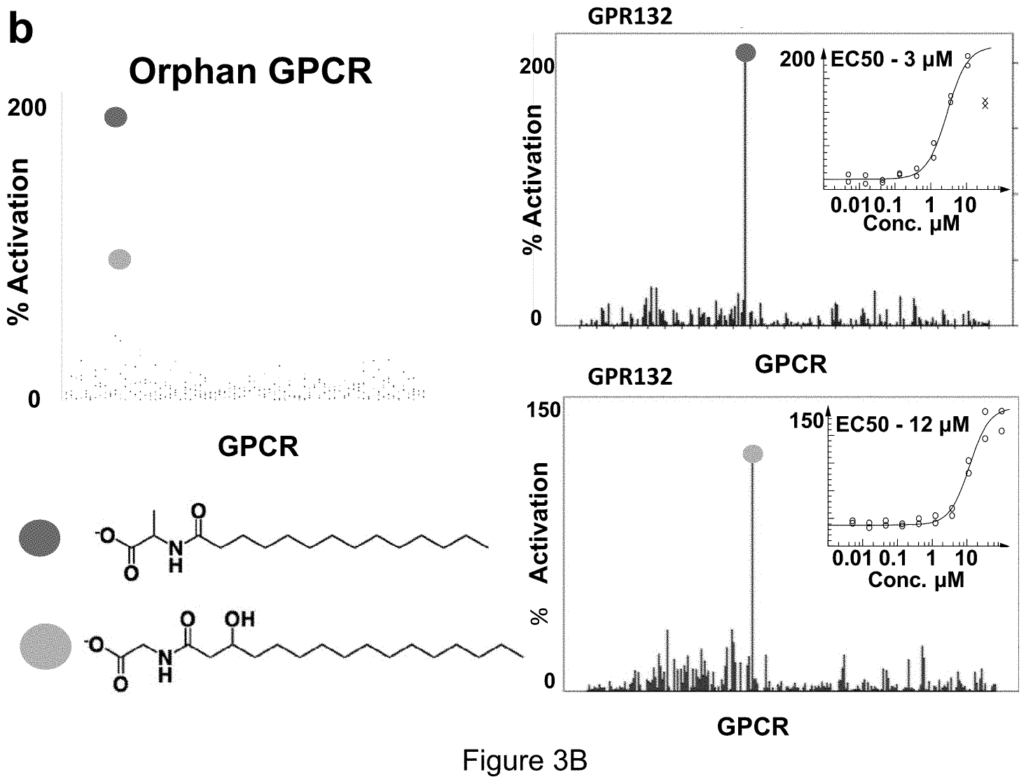

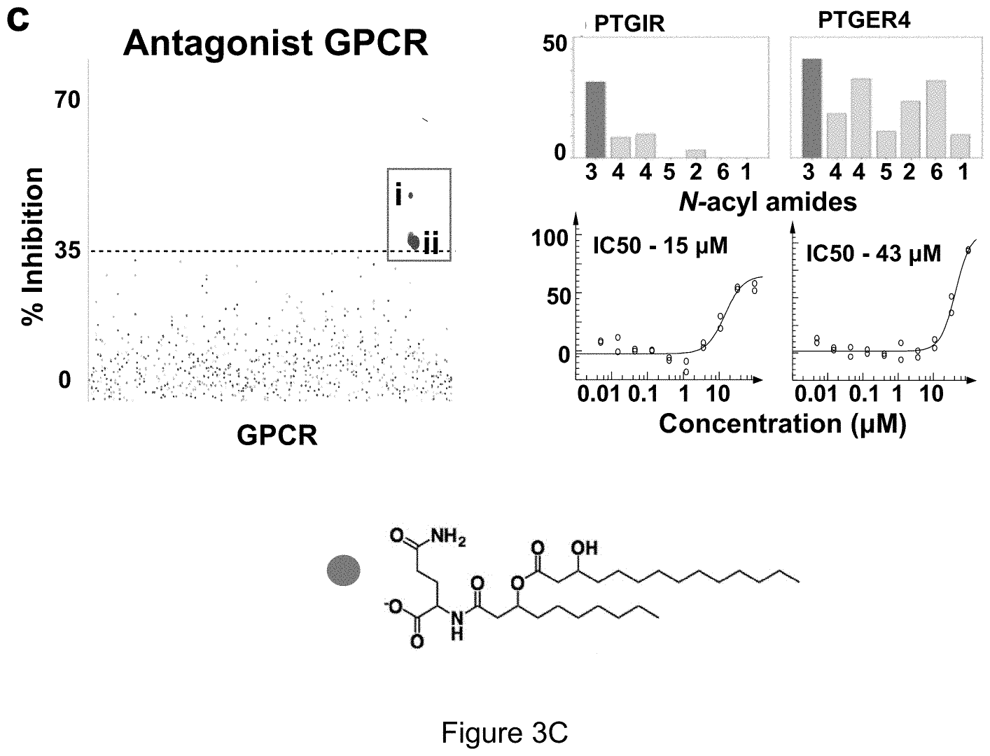

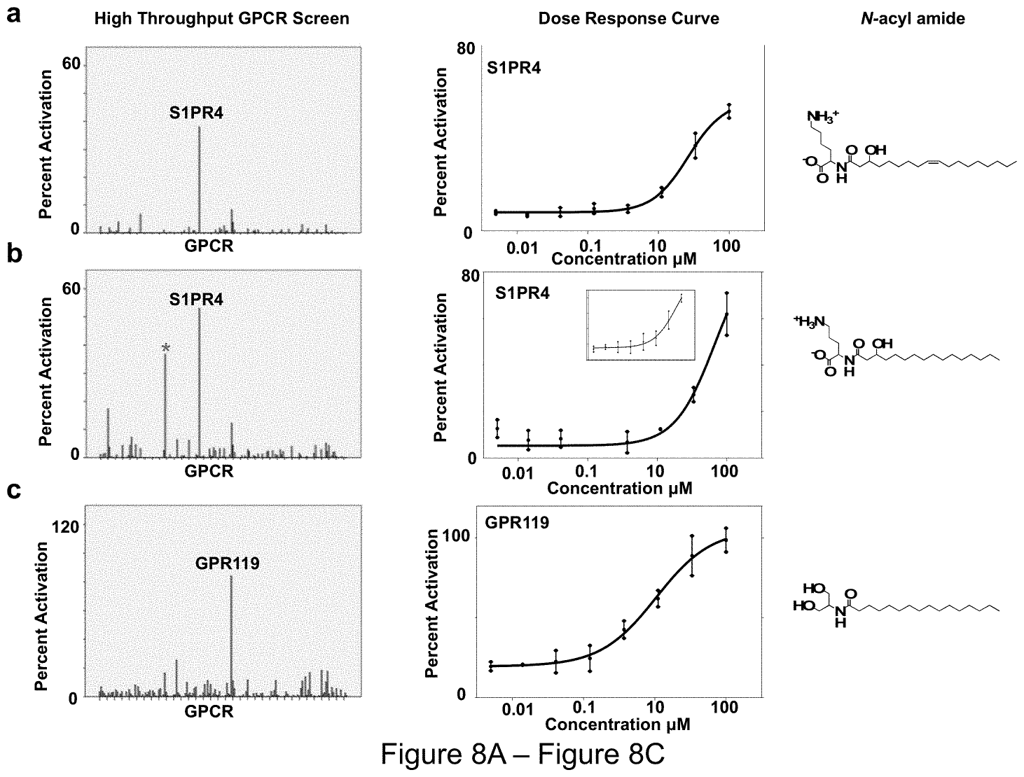

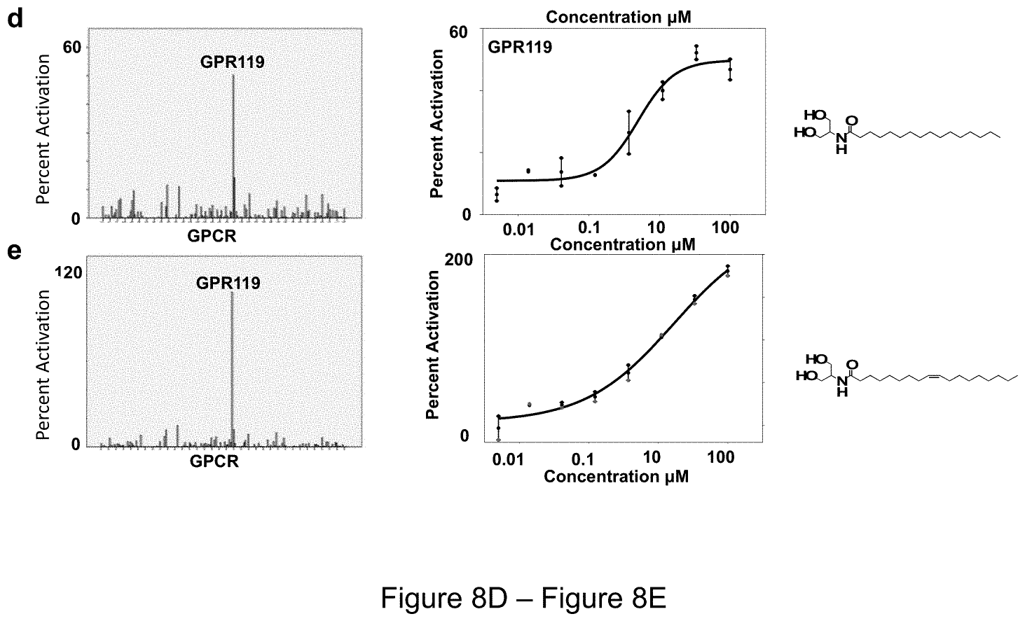

The major N-acyl amide analog isolated from each family was assayed for agonist and antagonist activity against a panel of 240 human GPCRs (FIG. 3 and FIG. 8). The strongest observed agonist interactions were: activation of GPR119 by N-palmitoyl serinol (EC50 9 μM), activation of sphingosine-1-phosphate receptor 4 (S1PR4) by N-3-hydroxypalmitoyl ornithine (EC50 32 μM) and activation of G2A by N-myristoyl alanine (EC50 3 μM). The maximal activation of GPR119 and S1PR4 by the bacterial N-acyl amides was similar to the endogenous ligand (GPR119 100%, S1PR4 80%). Interactions between the bacterial N-acyl amides and GPCRs were also specific (FIG. 3A and FIG. 3B). In each survey experiment no other GPCRs reproducibly showed greater than 30% activation relative to the endogenous ligands. The strongest antagonist activities were observed against two prostaglandin receptors, PTGIR and PTGER4 (FIG. 3C, PTGIR IC50 15 μM, PTGER4 IC50 43 μM). Interestingly, PTGIR was specifically antagonized by N-acyloxyacyl glutamine, while PTGER4 was antagonized by N-acyloxyacyl glutamine as well as other N-acyl amides (FIG. 3C(i) and FIG. 3C(ii)).

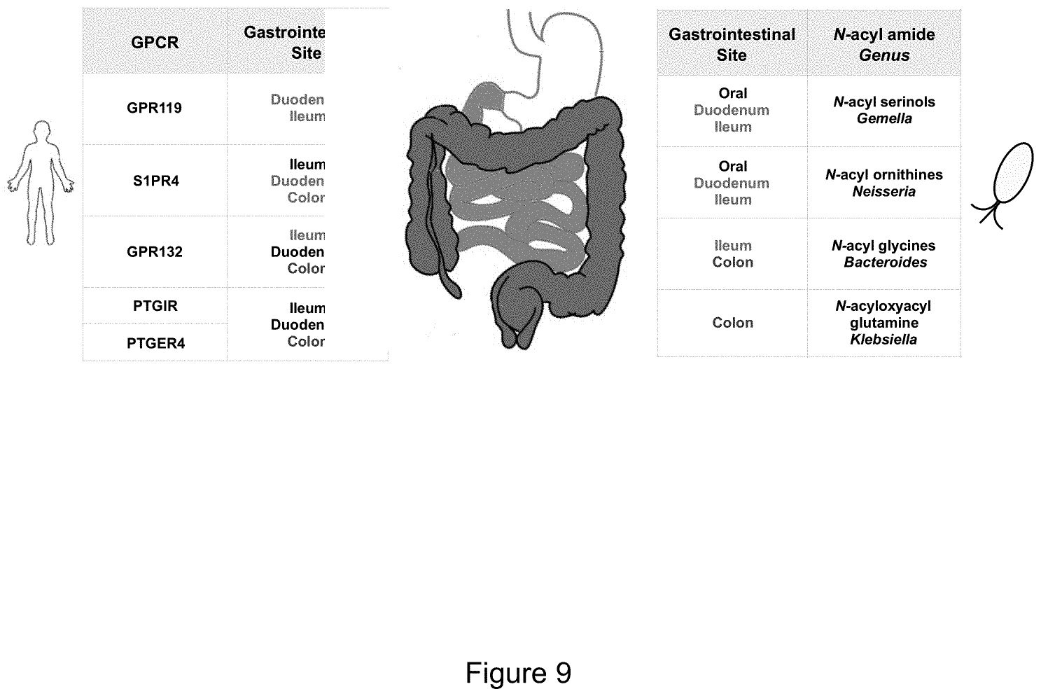

Based on data from the Human Protein Atlas (HPA) and Immunological Genome Project (ImmGen), GPCRs targeted by human microbial N-acyl amides are localized to the gastrointestinal tract and its associated immune cells. In mouse models, this collection of gastrointestinal tract localized GPCRs have been reported to affect diverse mucosal functions including metabolism (GPR119), immune cell differentiation (S1PR4, PTGIR, PTGER4), immune cell trafficking (S1PR4, G2A) and tissue repair (PTGIR) (Flock et al., 2011, Endorinol 152:374-83; Schulze et al., 2011, FASEB J 25:4024-36; Le et al., 2001, Immunity 14:561-71; Konya et al., 2013, Pharmacol Ther 138:485-502; Kabashima et al., 2013, J Clin Invest 109:883-93; Manieri et al., 2015, J Clin Invest 125:3606-18). It is not possible at this time to look for co-localization of GPCR and hm-NAS gene expression in specific gastrointestinal niches, as neither the HMP nor the HPA are sufficiently comprehensive in their survey of human body sites. Nonetheless, 16S and metagenomic deep sequencing studies link bacteria containing hm-NAS genes or hm-NAS genes themselves to specific locations in the gastrointestinal tract where GPCRs of interest are expressed (FIG. 9).

Bacterial and Human GPCR Ligands Share Structure and Function

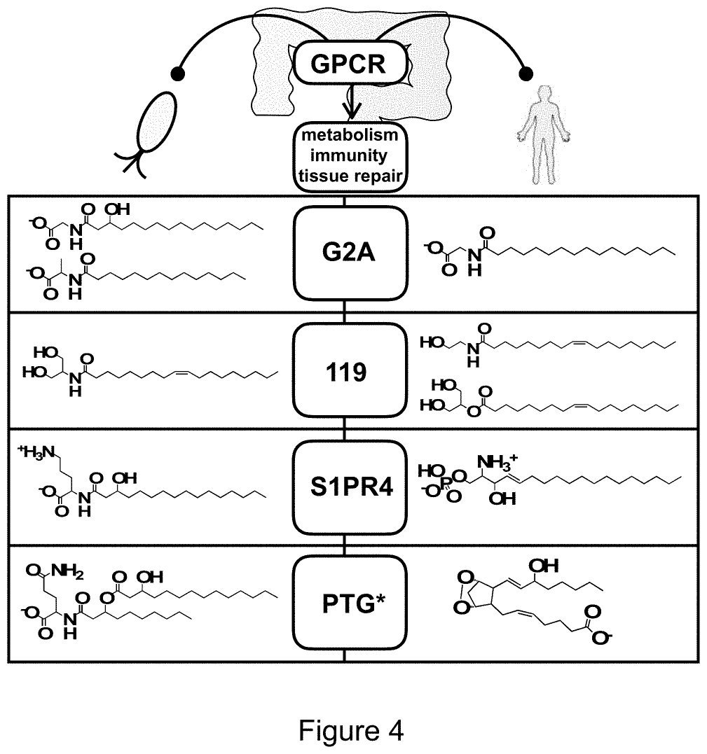

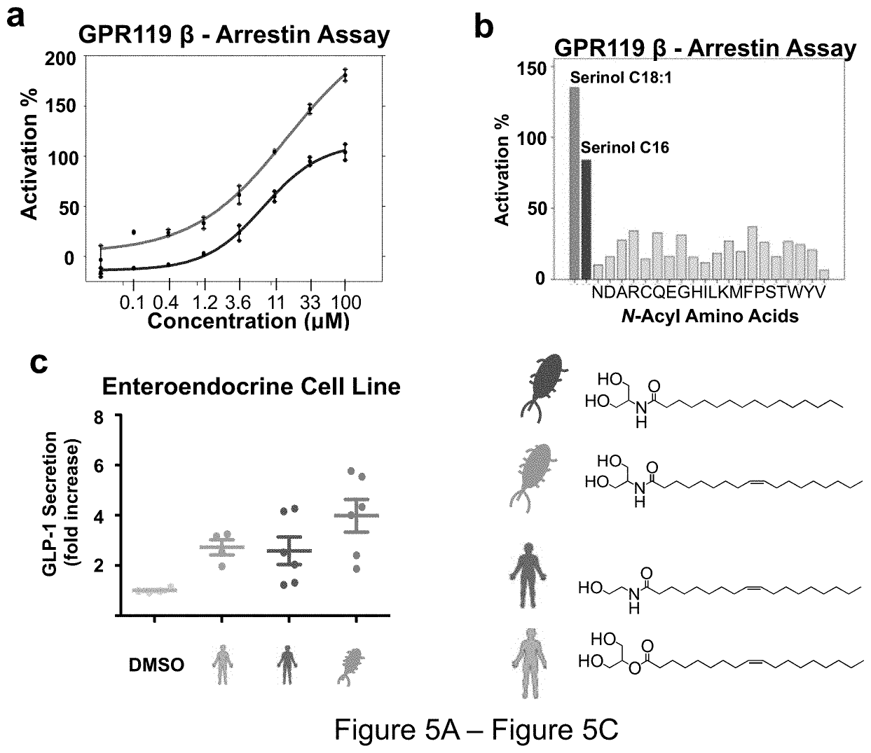

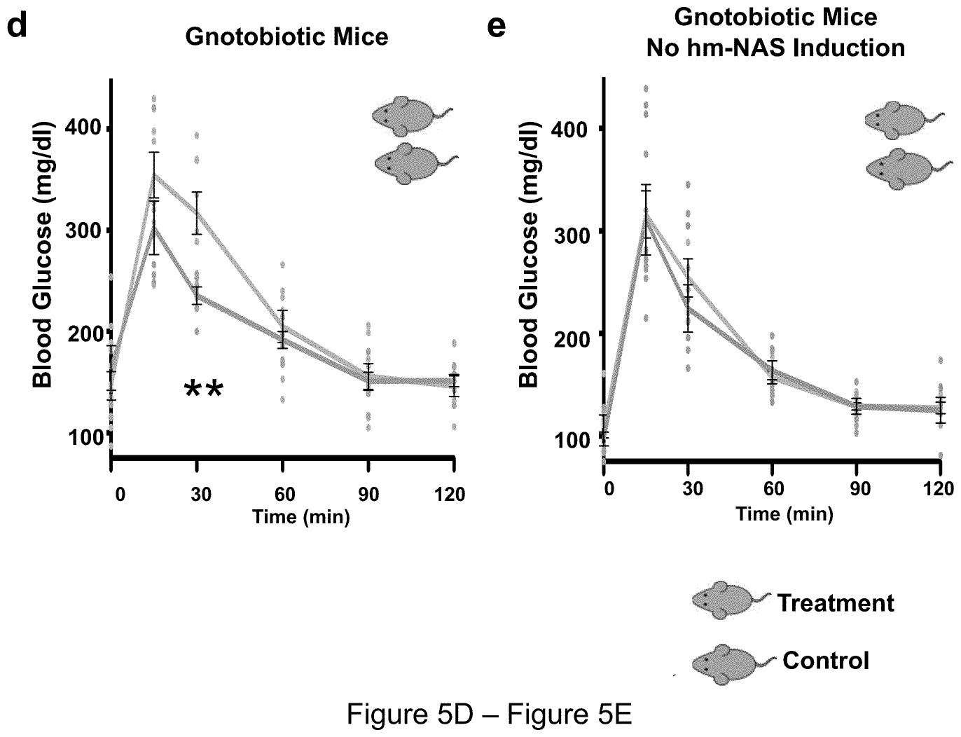

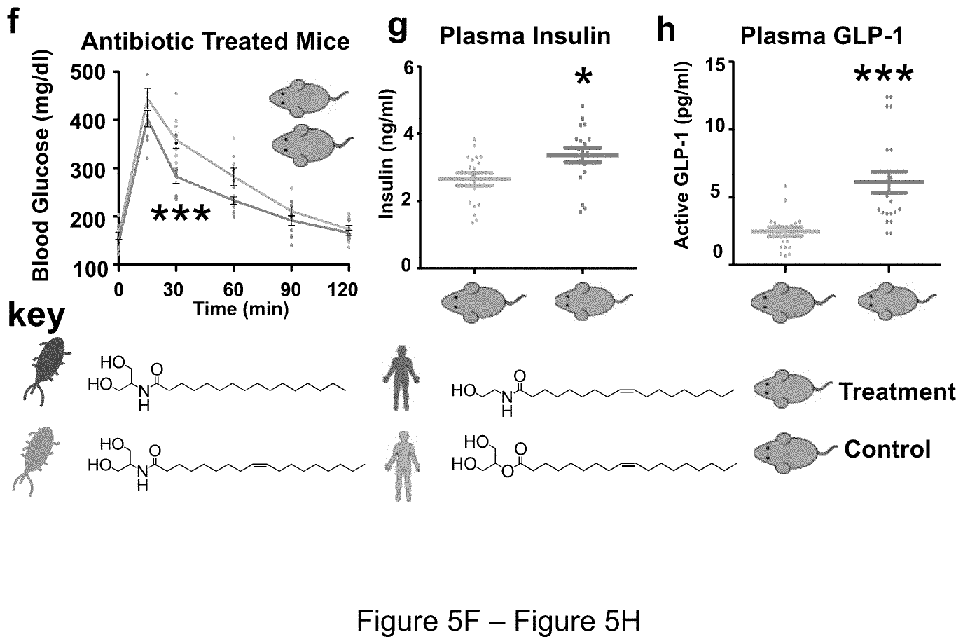

Human microbiota-encoded N-acyl amides bear striking structural similarity to endogenous GPCR-active ligands (FIG. 4, FIG. 5). The clearest overlap in structure and function between commensal N-acyl amides and human GPCR-active ligands is for the endocannabinoid receptor GPR119 (FIG. 4 and FIG. 5). Endogenous GPR119 ligands include the N-acyl amide oleoylethanolamide (OEA) (human) and the dietary lipid derivative 2-oleoyl glycerol (2-OG). Both the palmitoyl and oleoyl analogs of N-acyl serinol were isolated, the latter of which only differs from 2-OG by the presence of an amide instead of an ester and from OEA by the presence of an additional ethanol substituent. N-oleoyl serinol is a similarly potent GPR119 agonist compared to the endogenous ligand OEA (EC50 12 μM vs. 7 μM) but elicits almost a 2-fold greater maximum GPR119 activation (FIG. 5A). N-palmitoyl derivatives of all 20 natural amino acids were synthesized and none activated GPR119 by more than 37% relative to OEA (FIG. 5B). The generation of a potent and specific long-chain N-acyl-based GPR119 ligand therefore necessitates a more complex biosynthesis than the simple N-acylation of an amino acid as is commonly seen for characterized NAS enzymes. In this case, the biosynthesis of N-acyl serinols is achieved through the coupling of an NAS domain with an aminotransferase that is predicted to generate serinol from glycerol (FIG. 7).

The endogenous agonist for S1PR4, sphingosine-1-phosphate (S1P) and the N-3-hydroxypalmitoyl ornithine/lysine family of bacterial agonists share similar head group charge distribution patterns. S1P is a significantly more potent agonist (EC50 0.09 μM vs. EC50 32 μM) however, the bacterial agonists are more specific for S1PR4. The bacterial N-3-hydroxypalmitoyl ornithine did not activate S1PR1, 2, or 3 in the GPCR screen, whereas S1P activates all four members of the S1P receptor family tested.

No direct comparison could be made between the microbiota-derived and endogenous ligands for PTGIR or PTGER4, as there are no known endogenous antagonists for these receptors. Many human GPCRs remain orphan receptors lacking known endogenous ligands. Ligands for at least some of these receptors will undoubtedly be found among the small molecules produced by the human microbiota. G2A is an orphan receptor and therefore does not have a well-defined endogenous agonist, although it has been reported to respond to lysophosphatidylcholine (Khan et al., 2010, Biochem J 432:35-45; Kabarowski et al., 2009, Prostaglandins Other Lipid Mediat 89:73-81). The bacterial metabolites N-3-hydroxypalmitoyl glycine (commendamide) and N-palmitoyl alanine, both activate G2A. Mammals produce N-palmitoyl glycine, which differs from commendamide by the absence of the β-hydroxyl and based on the synthetic N-acyl studies activates G2A (Rimmerman et al., 2008, Mol Pharmacol 74:213-24).

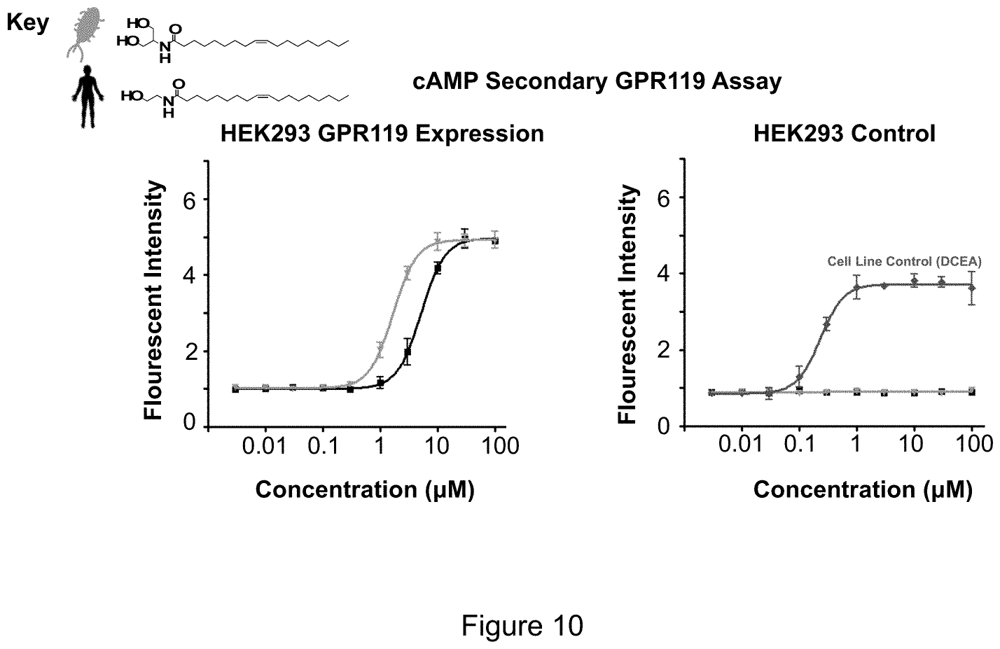

GPR119 is the most extensively studied of the GPCRs activated by bacterial N-acyl amides (FIG. 4). Mechanisms that link endogenous GPR119 agonists (OEA, 2-OG) to changes in host phenotype are well defined as a result of the exploration of GPR119 as a therapeutic target for the treatment of diabetes and obesity. In fact, synthetic small molecule GPR119 agonists are in clinical trials as treatment for both diseases (Ritter et al., 2016, J Med Chem 59:3579-92; Nunez et al., 2014, PLoS One 9:e92494; Ha et al., 2014, Arch Pharm Res 37:671-8; Katz et al., 2012, Diabetes Obes Metab 14:709-16). GPR119 agonists are thought to affect diverse metabolic functions, primarily glucose homeostasis but also gastric emptying and appetite, in vivo through GPR119-dependent hormone release from enteroendocrine cells (GLP-1, GIP, PYY) and pancreatic β-cells (insulin) (Flock et al., 2011, Endorinol 152:374-83; Overton et al., 2006, Cell Metab 3:167-75; Chu et al., 2007, Endocrinol 148:2601-9; Chu et al., 2008, Endocrinol 149:2038-47; Lauffer et al., 2009, Diabetes 58:1058-66; Serrano et al., 2011, Neuropharmacol 60:593-601). Murine enteroendocrine GLUTag cells have been used as a model system for measuring the ability of potential GPR119 agonists to induce GLP-1 release. When administered to GLUTag cells at equimolar concentrations, microbiota encoded N-oleoyl serinol or the endogenous ligands OEA and 2-OG induced GLP-1 secretion to the same magnitude (FIG. 5C). To provide an orthogonal measurement of GPR119 activation by N-acyl serinols, HEK293 cells were stably transfected with a GPR119 expression construct. Both OEA and N-oleoyl serinol increased cellular cAMP concentrations in a GPR119 dependent fashion (FIG. 10).

Bacteria Engineered to Produce N-acyl Serinols Alter Glucose Homeostasis in Mice

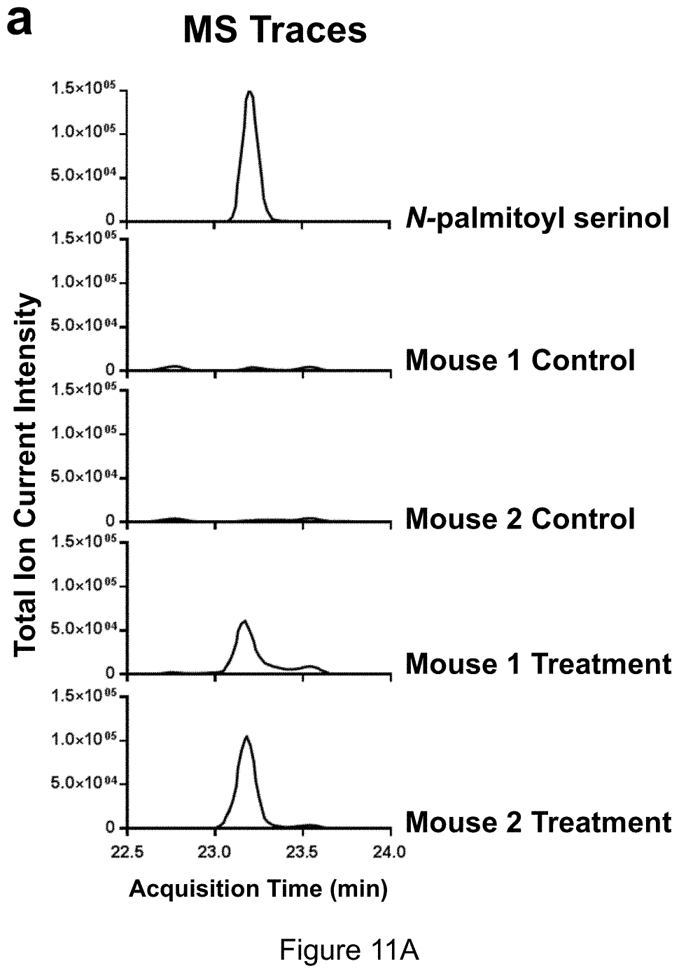

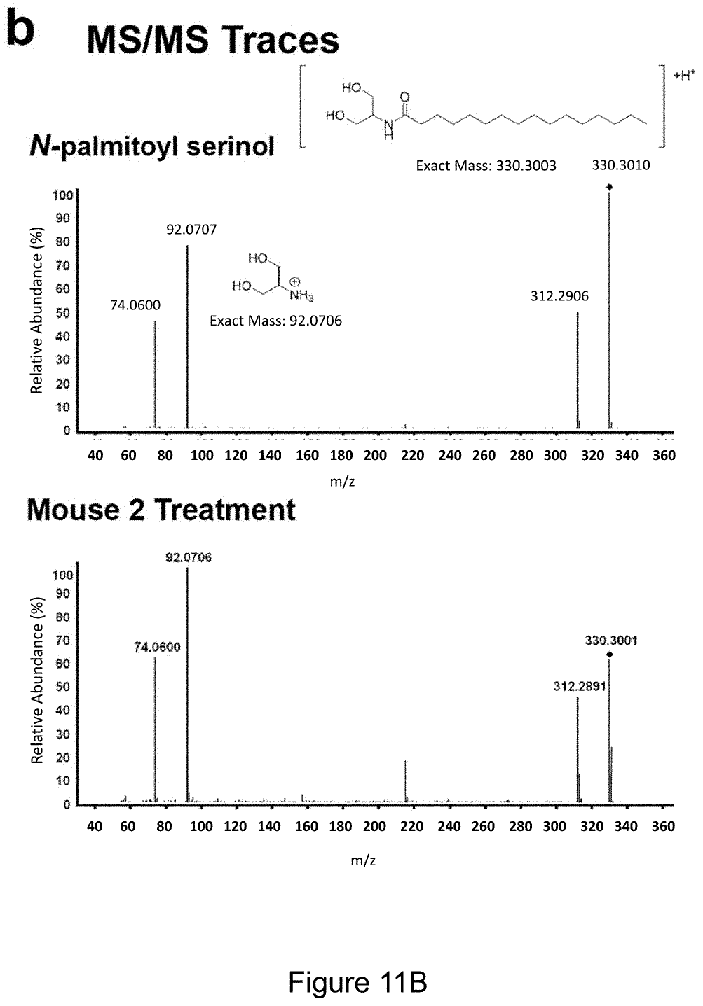

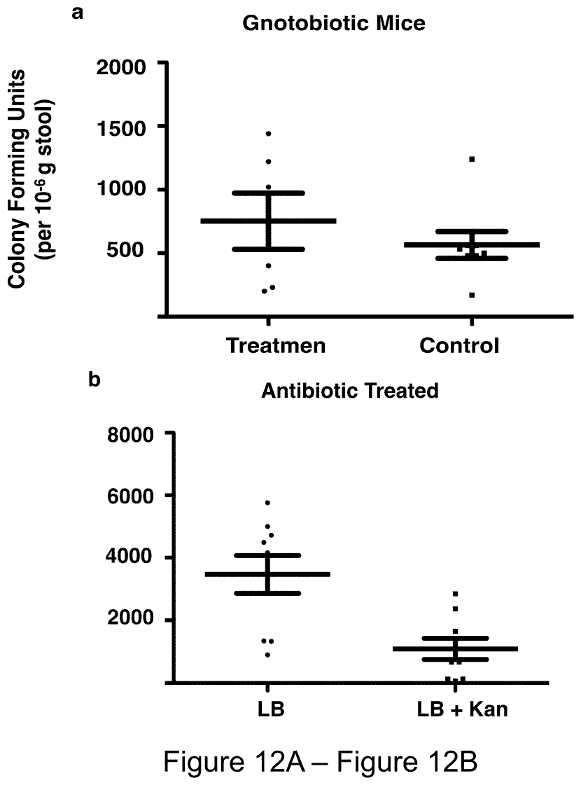

The functional overlap between endogenous and bacterial metabolites suggested to us that bacteria expressing microbiota-encoded GPR119 ligands might elicit host phenotypes that mimic those induced by eukaryotic ligands. Endogenous and synthetic GPR119 ligands have been associated with changes in glucose homeostasis that are relevant to the etiology and treatment of diabetes and obesity including a study where mice were orally administered bacteria engineered to produce a eukaryotic enzyme that increases endogenous GPR119 ligand (OEA) precursors (Flock et al., 2011, Endorinol 152:374-83; Overton et al., 2006, Cell Metab 3:167-75; Chu et al., 2007, Endocrinol 148:2601-9; Chu et al., 2008, Endocrinol 149:2038-47; Lauffer et al., 2009, Diabetes 58:1058-66; Serrano et al., 2011, Neuropharmacol 60:593-601; Chen et al., 2014, J Clin Invest 124:3391-406). The metabolic effect of the endogenous GPR119 ligands is believed to occur at the intestinal mucosa as the delivery of OEA intravenously fails to lower blood glucose in mice during an oral glucose tolerance test (OGTT). Consequently, it was sought to determine whether mice colonized with bacteria engineered to produce human microbiota N-acyl serinols would exhibit predictable host phenotypes. Gnotobiotic mice were colonized with E. coli engineered to express the N-acyl serinol synthase gene in an IPTG dependent manner. Control mice were colonized with E. coli containing an empty vector. Based on the number of colony forming units detected in fecal pellets both cohorts of mice were colonized to the same extent (FIG. 12). After one week of exposure to IPTG both cohorts were fasted overnight and subjected to an OGTT. At 30 minutes post challenge a statistically significant decrease in blood glucose levels was observed for the group colonized with E. coli expressing the N-acyl serinol synthase gene (FIG. 5D). MS analysis of metabolites present in cecal samples revealed the presence of N-acyl serinols in the treatment cohort but not in the control cohort (FIG. 11). After two weeks of withdrawing IPTG from the drinking water we no longer observed a difference in blood glucose between the two cohorts in an OGTT (FIG. 5E).

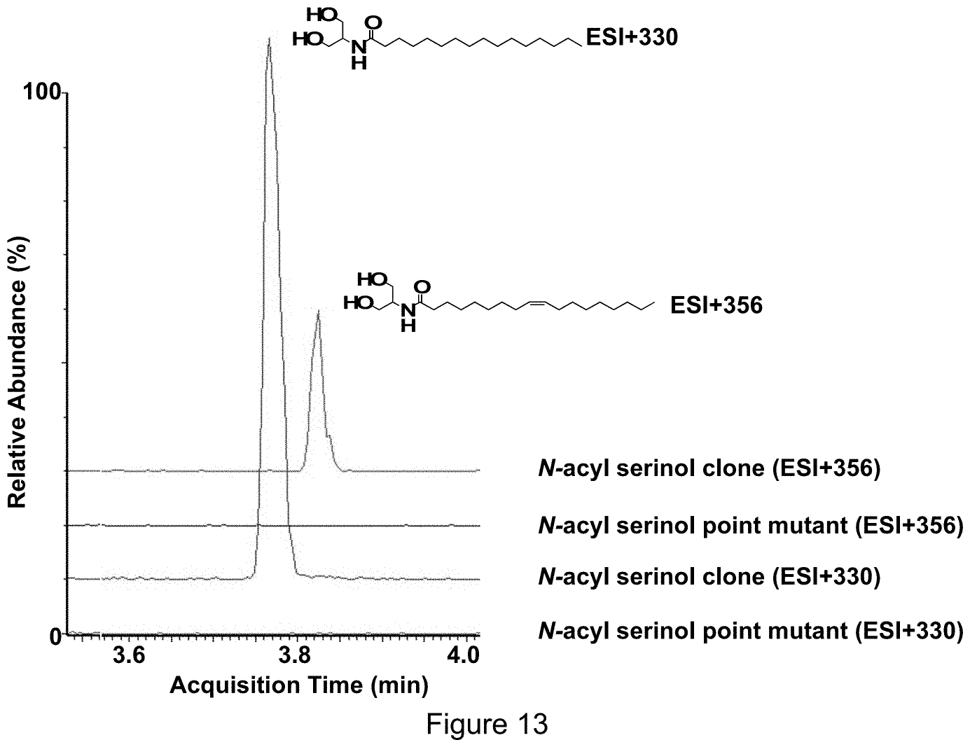

To further explore the metabolic phenotype induced by N-acyl serinols the OGTT experiment was repeated in an antibiotic treated mouse model. In this study mice colonized with E. coli expressing an active N-acyl serinol synthase were compared to mice colonized with E. coli expressing an NAS point mutant (FIG. 13, p.Glu91Ala) that no longer produced N-acyl serinols. In this model the glucose lowering effect of colonization with N-acyl serinol producing E. coli remained significant (FIG. 5F). In the antibiotic treated mice we measured GLP-1 and insulin concentrations after glucose gavage. Both hormones were significantly increased in the treatment group compared to the control group (FIG. 5G, FIG. 5H). In all mouse models the observed correlation between hm-NAS gene induction and increased glucose tolerance is similar in magnitude to several studies with small molecule GPR119 agonists including glyburide, an FDA approved therapeutic for diabetes.

### DISCUSSION

The characterization of human microbial N-acyl amides, together with other investigations of the human microbiota, suggests that host-microbial interactions may rely heavily on simple metabolites built from the same common lipids, sugars, and peptides that define many human signaling systems (e.g., neurotransmitters, bioactive lipids, glycans) rather than on the more complex small molecules commonly produced by many soil bacteria. This is not surprising, as the genomes of the bacterial taxa common to the human gastrointestinal tract (e.g., Bacteroidetes, Firmicutes and Proteobacteria) are actually quite poor in gene clusters that encode for the production of complex secondary metabolites (e.g., polyketides, nonribosomal peptides, terpenes) as compared to soil bacteria (e.g., Actinomycetes) (Donadio et al., 2007, Nat Prod Rep 24:1073-109). It appears that biosynthesis of endogenous mammalian signaling molecules as well as those produced by the human microbiota may rely on the modest manipulation of primary metabolites. As a result, the structural conservation between metabolites used in host-microbial interactions and endogenous mammalian signaling metabolites may be a common phenomenon in the human microbiome. Evolutionarily, the convergence of bacterial and human signaling systems through structurally related GPCR ligands is not unreasonable as GPCRs are thought to have developed in eukaryotes to allow for structurally simple signaling molecules to regulate increasingly complex cellular interactions (Vaudry, 2014, J Mol Endocrinol 52:E1-2; Lovejoy, 2014, J Mol Endocrinol 52:T43-60; Hla, 2005, Prostaglandins Other Lipid Mediat 77:197-209). The structural similarities between human microbiota-encoded long-chain N-acyl amides and endogenous GPCR-active lipids may be indicative of a broader structural and functional overlap among bacterial and human bioactive lipids including other GPCR-active N-acyl amides, eiconasoids (prostaglandins, leukotrienes) and sphingolipids. While not wishing to be bound to any particular theory, it is possible that structural similarities between microbiota-encoded N-acyl amides and endogenous GPCR-active lipids may be indicative of a broader structural and functional overlap among bacterial and human bioactive lipids including other GPCR-active N-acyl amides, eiconasoids (prostaglandins, leukotrienes) and sphingolipids. Sphingolipid based signaling molecules may also be common in the human microbiome as prevalent bacterial species are known to synthesize membrane sphingolipids.

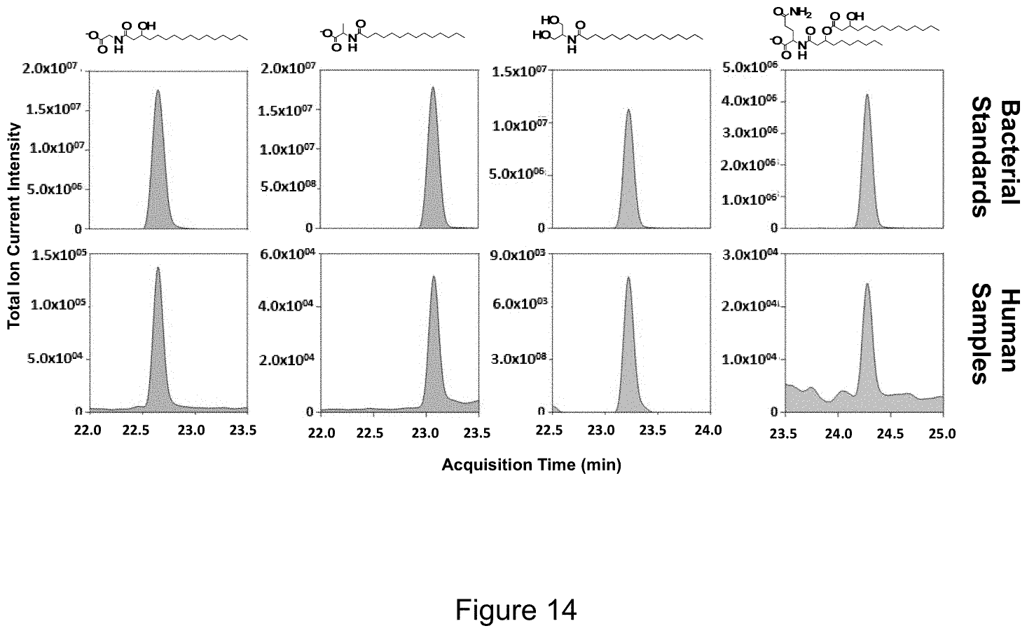

The GPCRs with which bacterial N-acyl amides were found to interact, GPR119, S1PR4, PTGER4 and PTGIR are all part of the same “lipid-like” GPCR gene family. The potential importance of this GPCR family to the regulation of host-microbial interactions is suggested by their localization to areas of gastrointestinal track enriched in bacteria that are predicted to synthesize GPCR ligands (FIG. 9). Lipid-like GPCRs have been shown to play roles in disease models that are correlated with changes in microbial ecology including colitis (S1PR4, PTGIR, PTGER4), obesity (GPR119), diabetes (GPR119), autoimmunity (G2A) and atherosclerosis (G2A, PTGIR). The fact that the expression of an N-acyl synthase gene in a gut colonizing bacterium is sufficient to alter host physiology suggests that the interaction between lipid-like GPCRs and their N-acyl amide ligands could be relevant to human physiology and warrants further study. By LCMS analysis we observed most of the microbiota encoded N-acyl amides reported here in human stool samples (FIG. 14). Further studies will be needed to better define the distribution and concentration of these metabolites throughout the gastrointestinal tract especially at the mucosa where the physiologic activity of these metabolites likely occurs. Interestingly, Gemella spp. predicted to encode N-acyl serinols are tightly associated with the small intestinal mucosa supporting this site as a potentially important location for N-acyl amide mediated interactions. As the mouse model system used here relies on induced expression of NAS genes it will also be important to understand how these genes are natively regulated.

Current strategies for treating diseases associated with the microbiome such as inflammatory bowel disease or diabetes are not believed to address the dysfunction of the host-microbial interactions that are likely part of the disease pathogenesis. Bacteria engineered to deliver bioactive small molecules produced by the human microbiota have the potential to help address diseases of the microbiome by modulating the native distribution and abundance of these metabolites. Regulation of GPCRs by microbiota-derived N-acyl amides is a particularly attractive therapeutic strategy for the treatment of human diseases as GPCRs have been extensively validated as therapeutic targets. As our mechanistic understanding of how human microbiota-encoded small molecules effect changes in host physiology grows, the potential for using “microbiome-biosynthetic-gene-therapy” to treat human disease by complementing small molecule deficiencies in native host-microbial interactions with microbiota derived biosynthetic genes should increase accordingly. The use of functional metagenomics to identify microbiota encoded effectors combined with bioinformatics and synthetic biology to expand effector molecule families provides a generalizable platform to help define the role microbiota-encoded small molecules play in host-microbial interactions.

The materials and methods are now described

Bioinformatics Analysis of Human N-acyl Synthase Genes

Protein sequences for members of the PFAM family 13444 Acetyltransferase (GNAT) domain (http://pfam.xfam.org/family/PF 13444) (n=689) were downloaded and corresponding gene sequences identified based on European Bioinformatics Institute (EBI) number. A multiple sequence alignment was performed using Clustal Omega (http://www.ebi.ac.uk/Tools/msa/clustalo/), generating a phylogenetic tree in Newick format with the “-guidetree-out” option. The 689 PFAM sequences were queried against the Human Microbiome Project (HMP) clustered gene index datasets and reference genome datasets with BLASTN (http://hmpdacc.org/HMGC/). The PFAM13444 sequences that aligned to a HMP gene [expectation (E) value <e⁻⁴⁰ and >70% identity] were identified and comprise the human N-acyl synthase (hm-NAS) gene dataset (143 hm-NAS genes). Reference genomes for 111/143 hm-NAS genes were identified (Table 4).

To determine the abundance of hm-NAS genes within microbiomes at specific human body sites, hm-NAS genes were queried against HMP whole metagenome shotgun sequencing data on a per body site basis (http://hmpdacc.org/HMASMI). Each hm-NAS gene was BLASTN searched against the non-redundant gene sets from the following body sites: buccal mucosa, posterior fornix, retroauricular crease (combined left and right), stool, supragingival plaque and tongue dorsum. These body sites were chosen because they contained sequence data from the largest number of unique patients (Human Microbiome Project Consortium, 2012, Nature 486:207-14). hm-NAS genes and highly similar genes in the HMP non redundant gene set (E-value <e⁻⁴⁰) were aligned to shotgun sequencing reads from each patient sample taken from different sites in the human microbiome. Aligned reads were normalized to hm-NAS gene length and sequencing depth of each dataset. The normalized count of the reads aligned to each hm-NAS gene or its highly similar gene from the HMP non redundant gene set were scaled [0-1] and color coded per body site, and added as concentric rings around the phylogenetic tree (FIG. 1A). To determine the variability and distribution of hm-NAS genes that correspond to specific N-acyl amide families 1-6 (FIG. 1) in the human microbiome normalized read counts for hm-NAS gene from each N-acyl amide family were plotted separately per body site as Reads per Kilobase of Gene Per Million Reads (RPKM) (FIG. 1C). The tree in FIG. 1 was plotted using graphlan (https://huttenhower.sph.harvard.edu/graphlan).

Analysis of Metatranscriptome Datasets

Two RNAseq datasets were identified with multiple patient samples taken from separate sites in the human microbiome (Franzosa et al., 2014, PNAS 3111:E2329-38; Peterson et al., 2014, Front Cell Infect Microbiol 4:108). One RNAseq dataset was part of the HMP (http://hmpdacc.org/RSEQ/) and generated from supragingival samples taken from twin pairs with and without dental caries. The second RNAseq dataset was generated from stool samples and compared different RNA extraction methods. Only samples labeled “whole” were used, which functioned as controls for the original study (Franzosa et al., 2014, PNAS 3111:E2329-38). Alignment of all hm-NAS genes to each dataset only identified hm-NAS genes from N-acyl amide family 1 and 2 in each of the RNAseq datasets (1 in stool, 2 in supragingival plaque). To explore whether hm-NAS gene expression might vary in patient samples two different analyses were performed. In the first analysis, a reference genomes was identified containing hm-NAS genes identical to those used in heterologous expression experiments for molecule families 1 and 2 (Bacteroides dorei for compound 1, Capnocytophaga ochracea for compound 2). RNAseq reads were aligned to all of the genes from each reference genome. Normalized RNAseq reads that aligned to each genome. For each genome the average per gene read density normalized for gene length was compared to the read density seen for the hm-NAS gene. The percentile of the normalized expression of each hm-NAS gene was then plotted (0 for not expressed, 1 for the most expressed) and compared between patient samples for each RNAseq dataset (FIG. 2A). In the second analysis the direct correlation between DNA and RNA abundance was determined for the stool metatranscriptome dataset for which DNA reads were also available.⁴¹ RNAseq and shotgun-sequenced DNA reads were aligned to the 15 hm-NAS genes from N-acyl amide family 1 that encoded for N-acyl glycines (Table 3). The reads were normalized (RPKM) and each hm-NAS gene from each patient sample was plotted as a single point with DNA and RNA read counts on the X and Y axis (FIG. 2B).

Heterologous Expression of PFAM13444 Genes in Escherichia coli The 43 hm-NAS genes examined by heterologous expression were codon optimized, appended with Ncol and Ndel sites at the N and C terminus respectively and synthesized by Gen9. Genes obtained from Gen9 were digested with Ndel and Ncol and ligated into the corresponding restriction sites in pET28c (Novagen). For heterologous purposes the resulting constructs were transformed into E. coli EC100 containing the T7 polymerase gene integrated into its genome (E. coli EC100:DE3). E. coli EC100:DE3 hm-NAS containing strains were inoculated into 10 ml of Luria-Bertani (LB) broth supplemented with kanamycin (50 μg/ml) and grown overnight (37° C. with shaking 200 rpm). One ml of overnight culture was used to inoculate 50 ml of LB supplemented with kanamycin (50 μg/ml) and isopropyl β-D-1-thiogalactopyranoside (IPTG) (25 μM). Cultures were incubated at 30° C. for 4 days with shaking (200 rpm). Each culture broth was extracted with an equal volume of ethyl acetate and the resulting crude extracts were dried in vacuo. Crude extracts were resuspended in 50 μL of methanol and analyzed by reversed phase HPLC-MS (Waters XBridge™, 4.6×150 mm) using a binary solvent system (A/B solvent of water/acetonitrile with 0.1% formic acid: 10% B isocratic for 5 minutes, gradient 10% to 100% B over 25 minutes). Clone specific metabolites encoded by each hm-NAS gene were identified by comparing experimental extracts extracts prepared from cultures of E. coli EC100:DE3 transformed with an empty pET28c vector.

N-acyl Amide Isolation and Structure Determination:

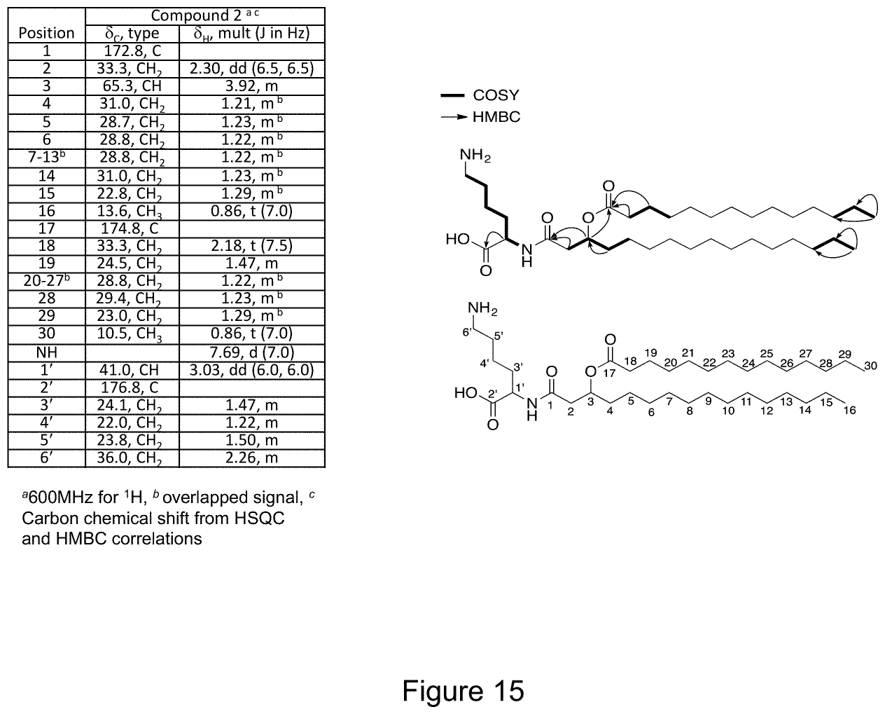

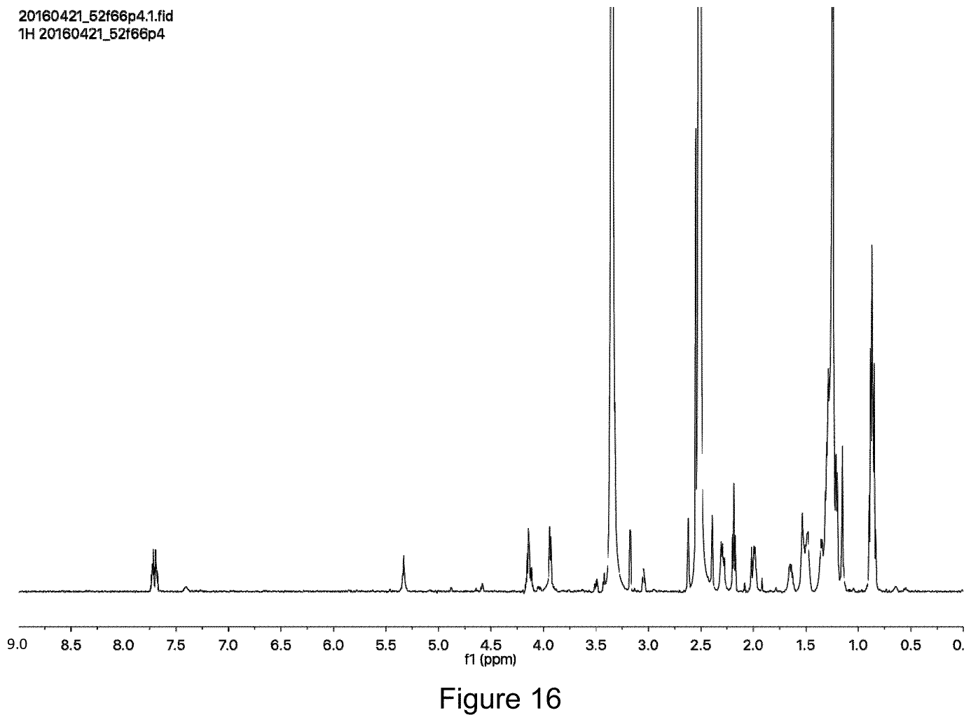

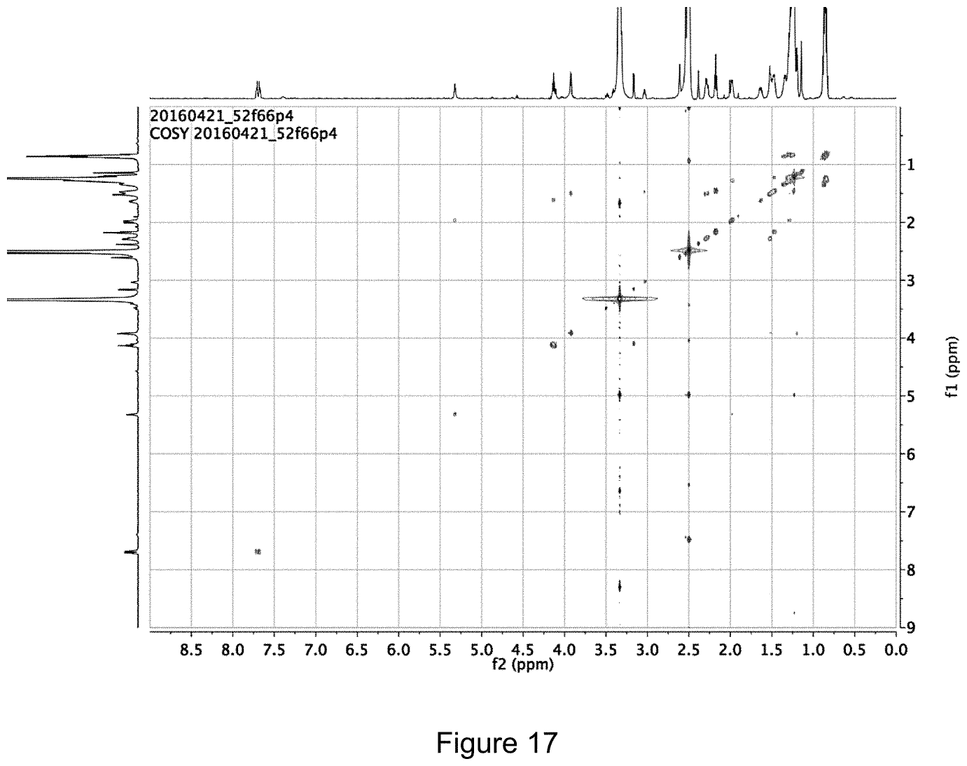

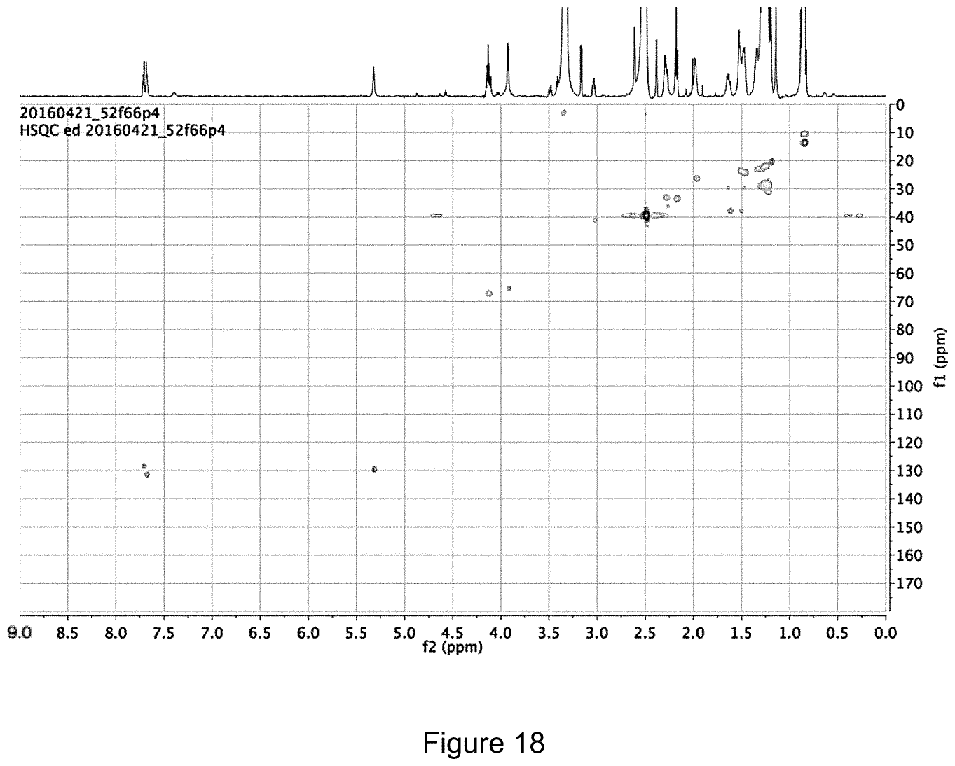

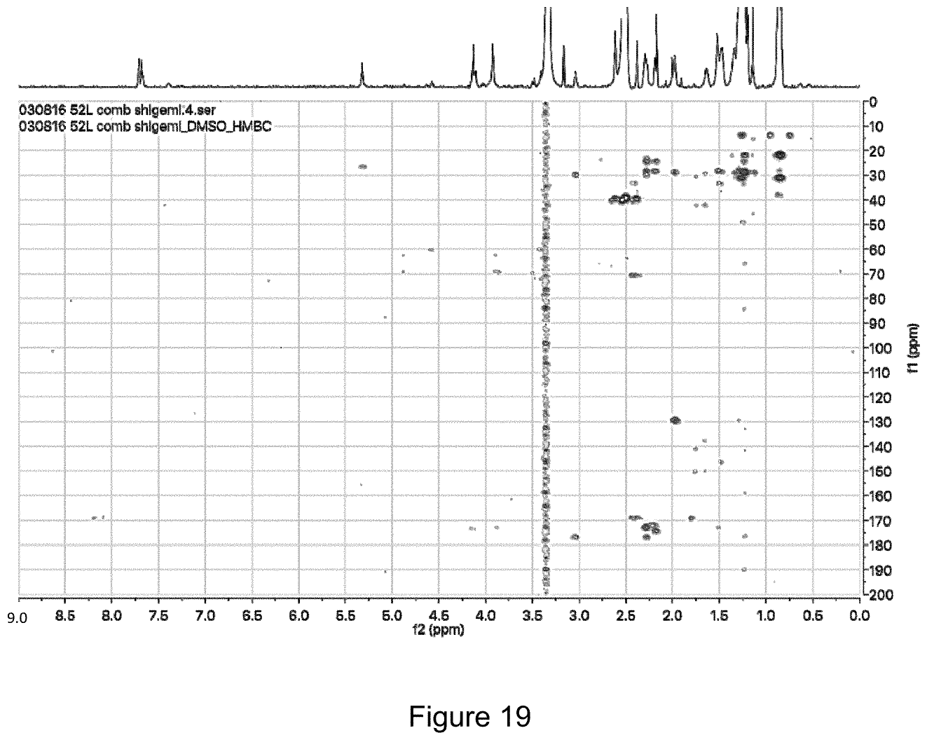

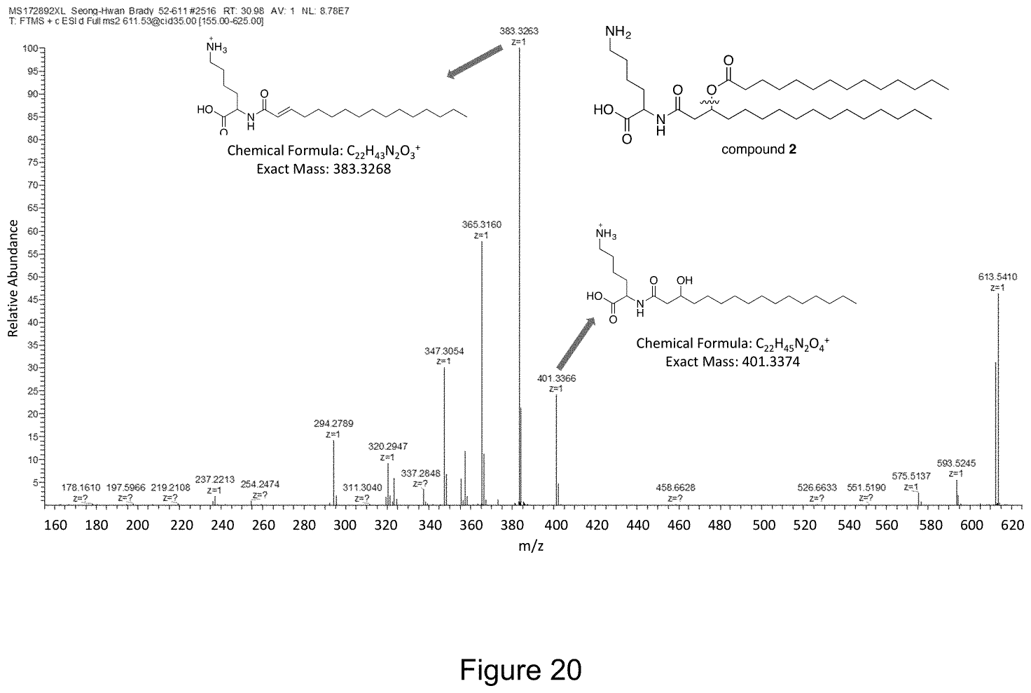

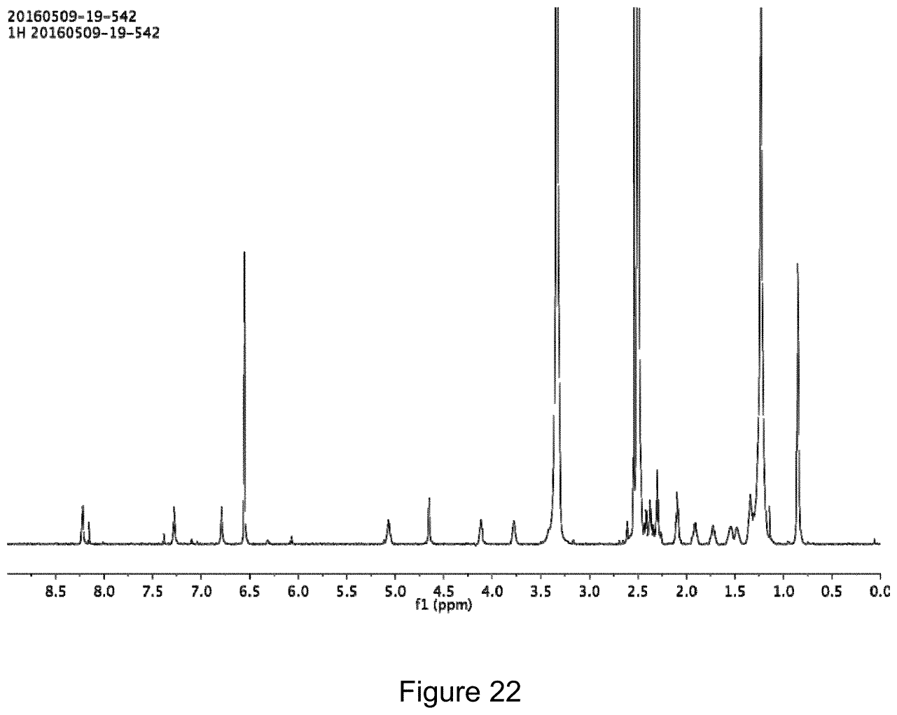

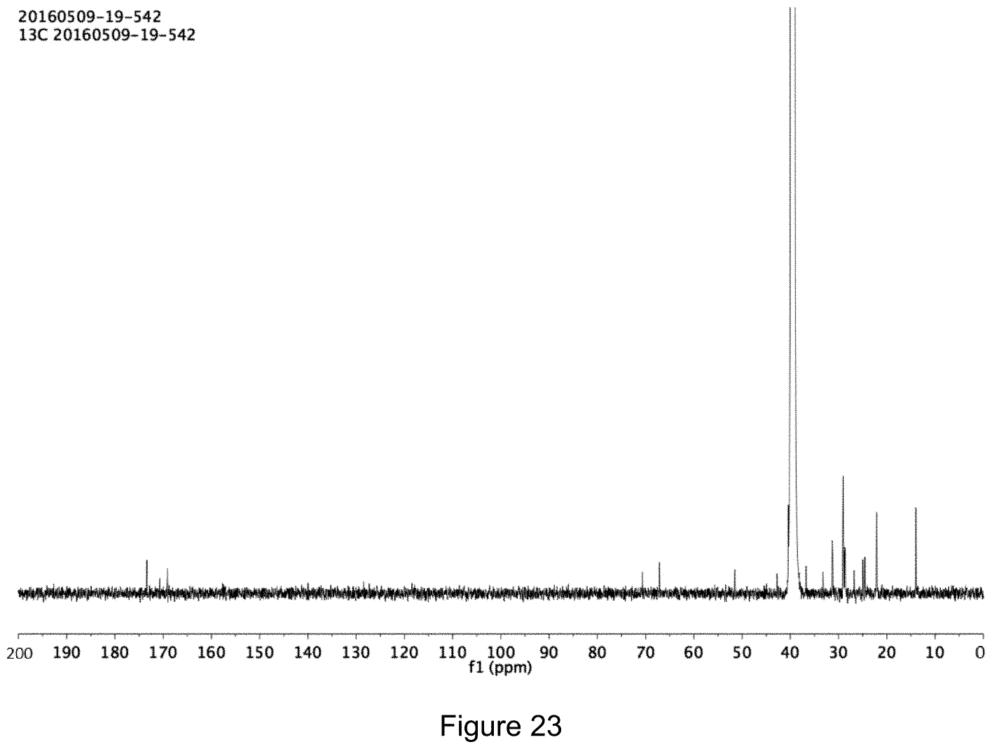

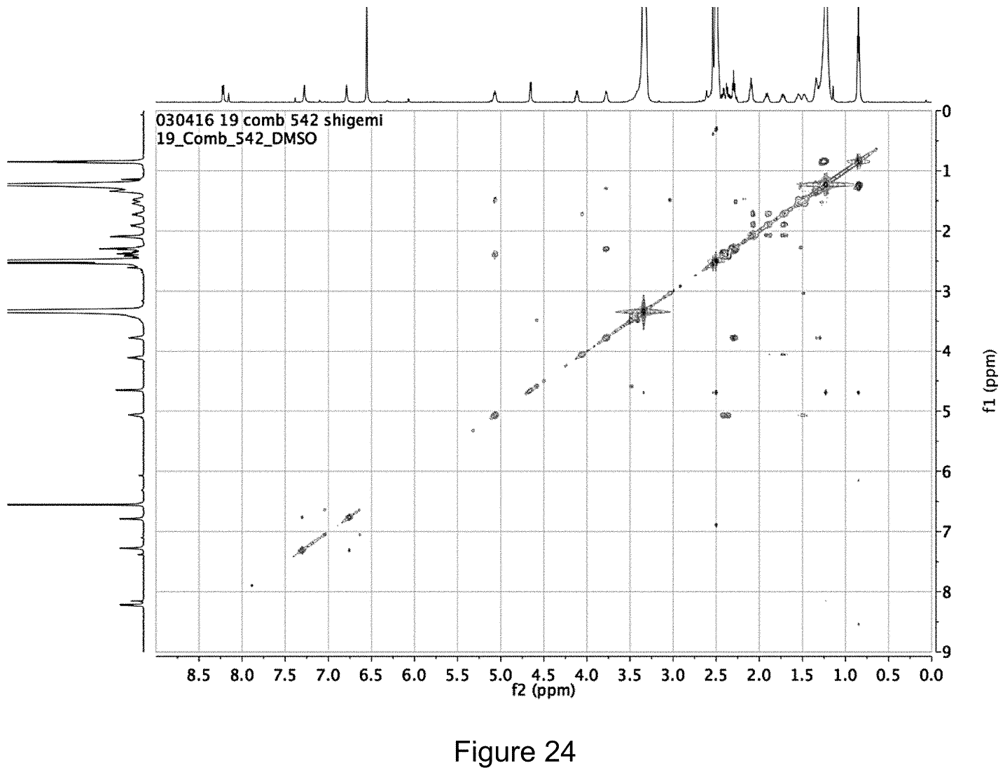

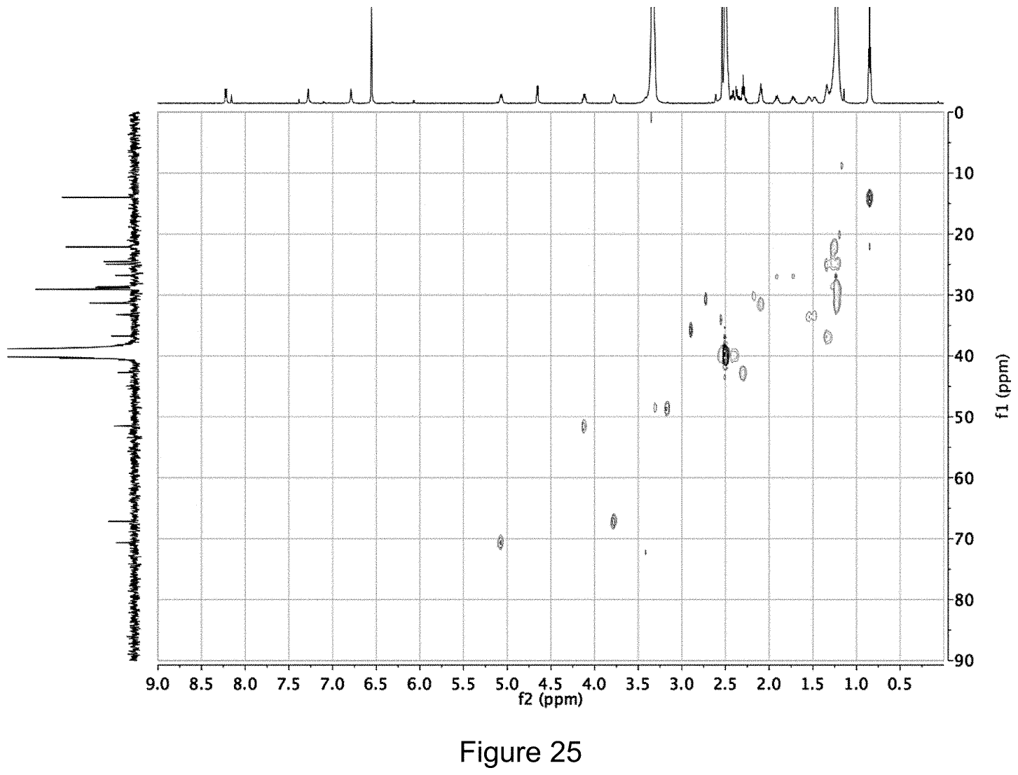

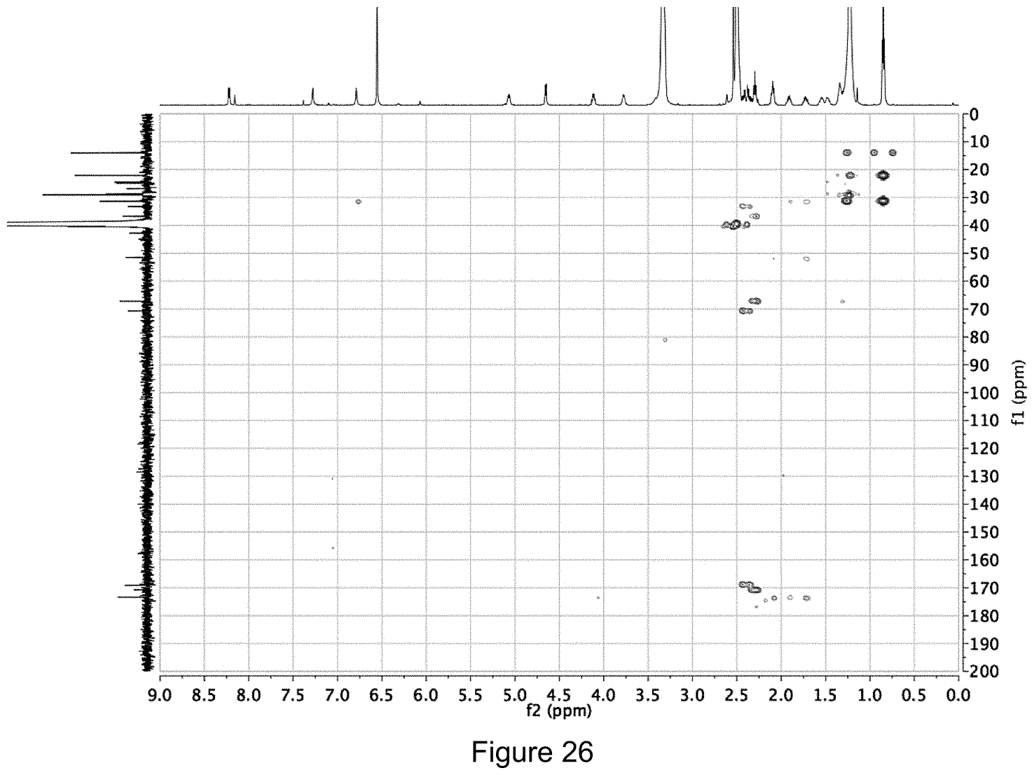

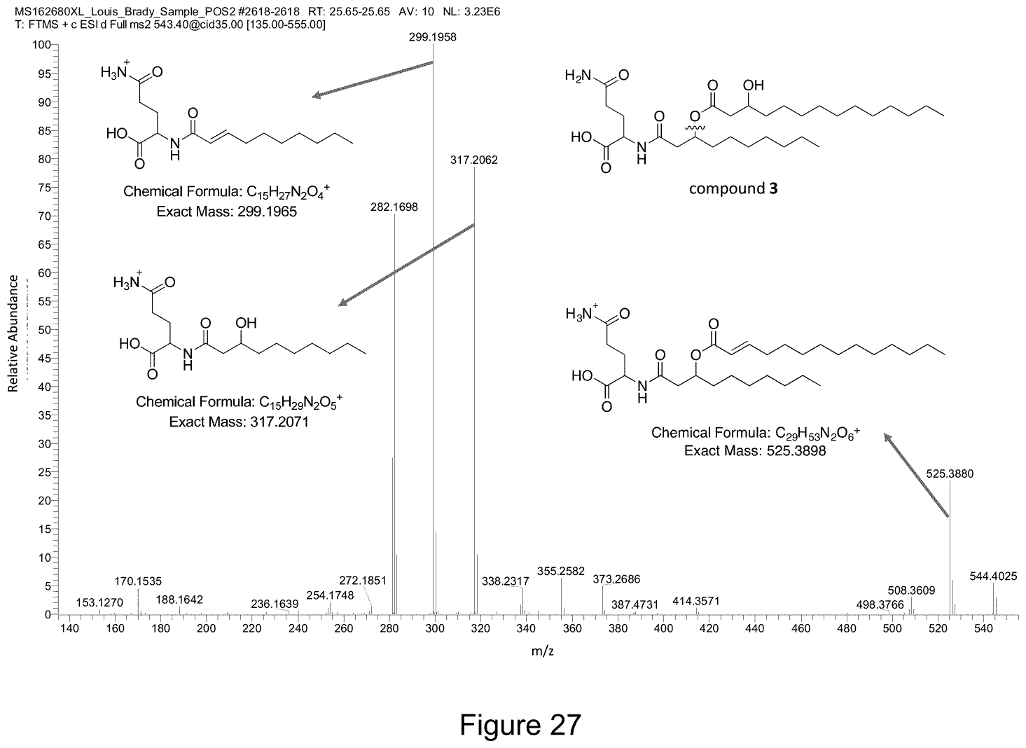

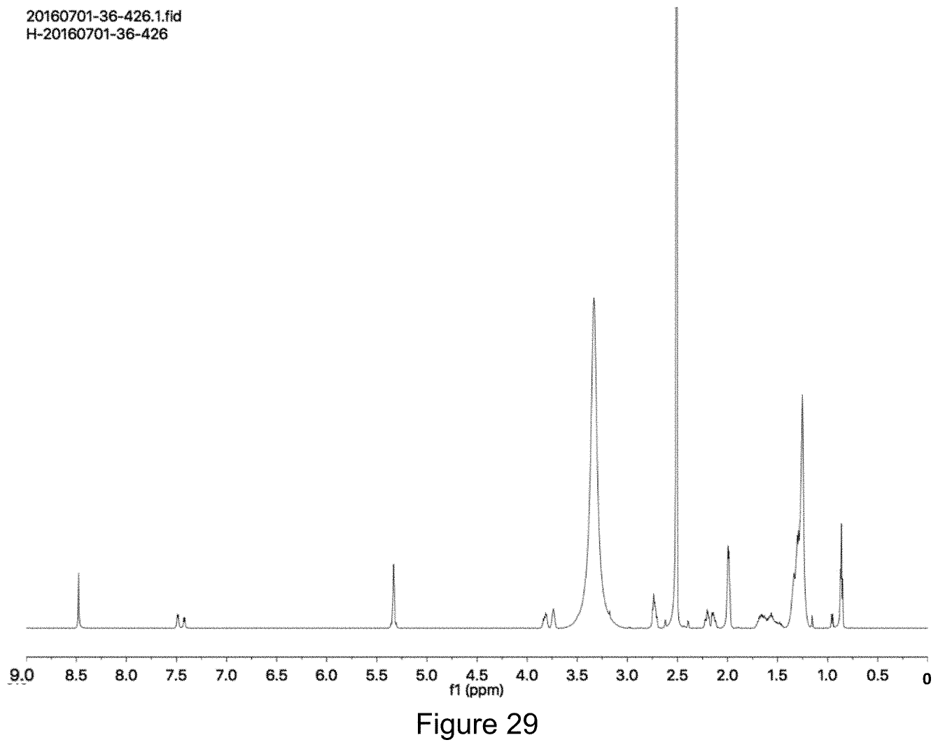

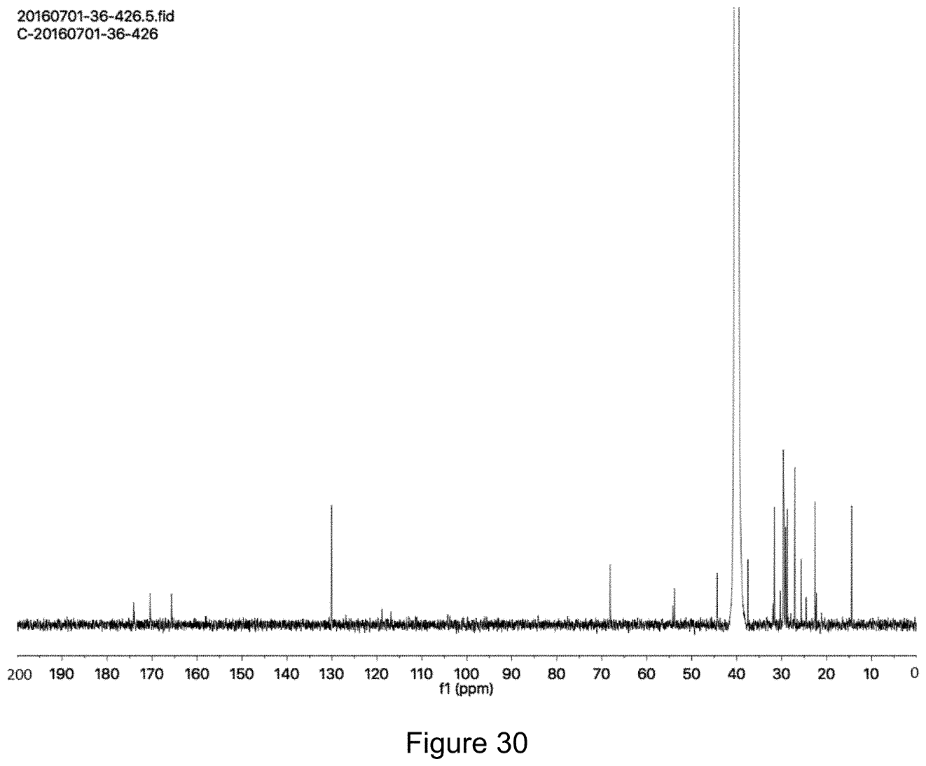

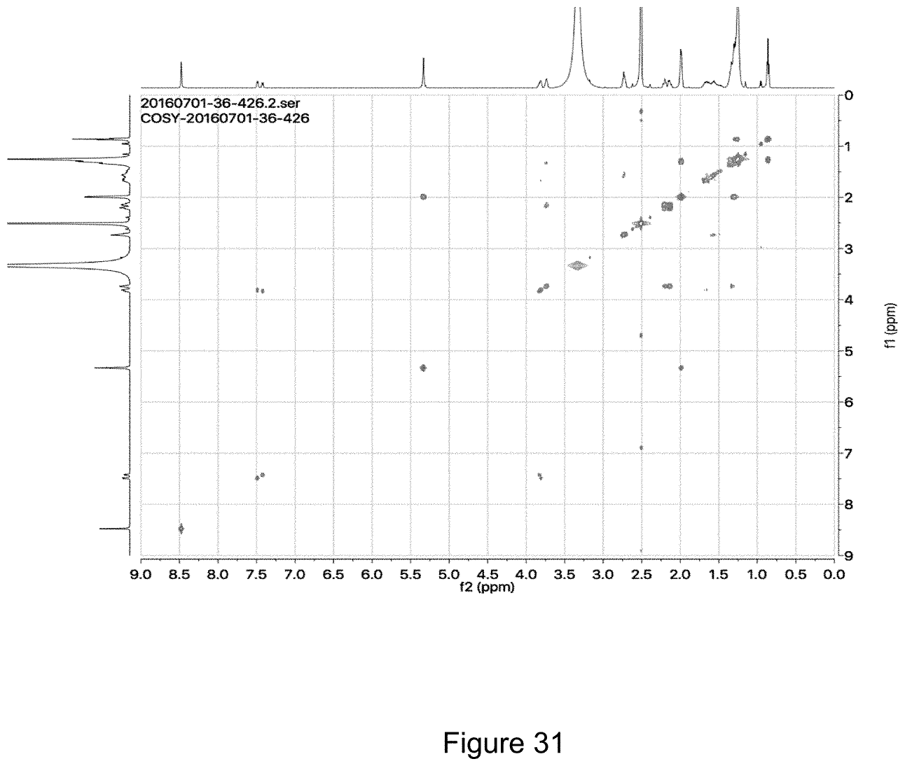

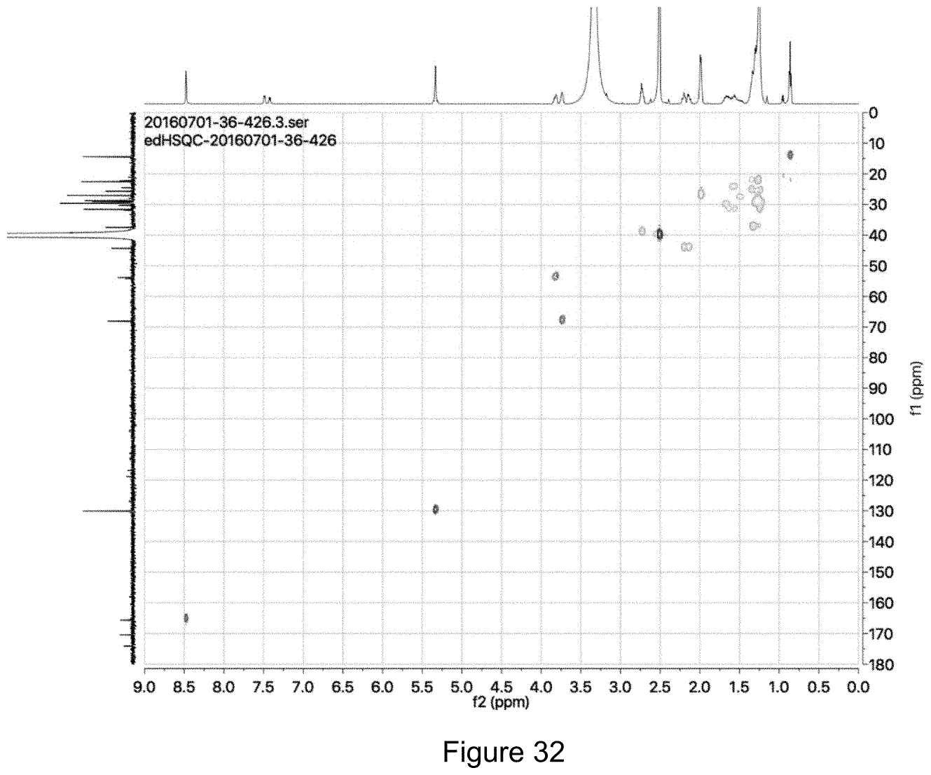

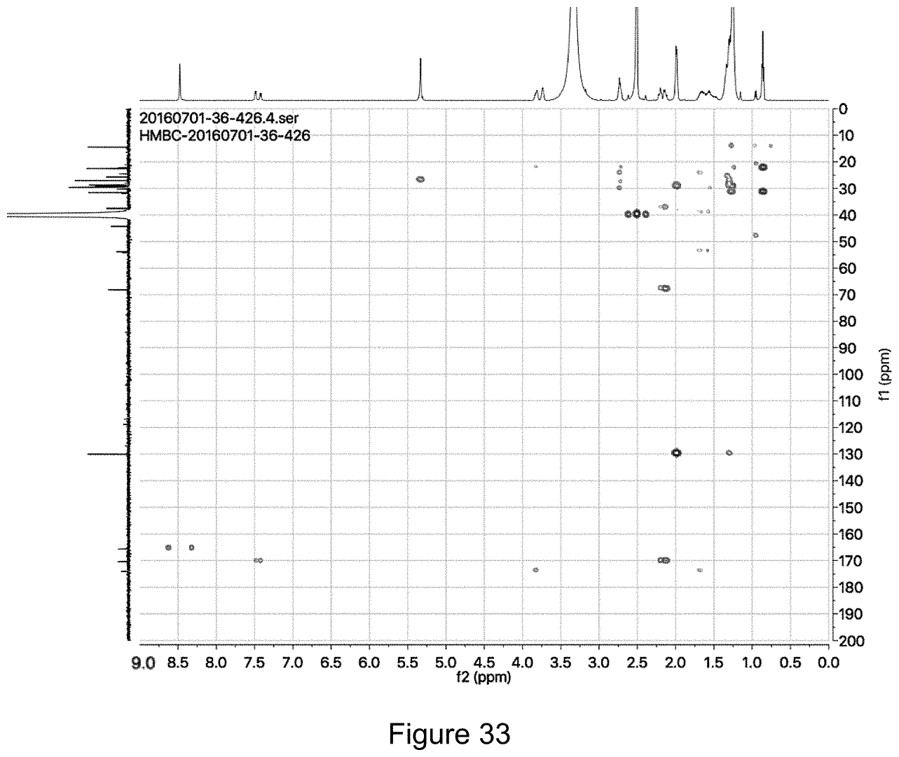

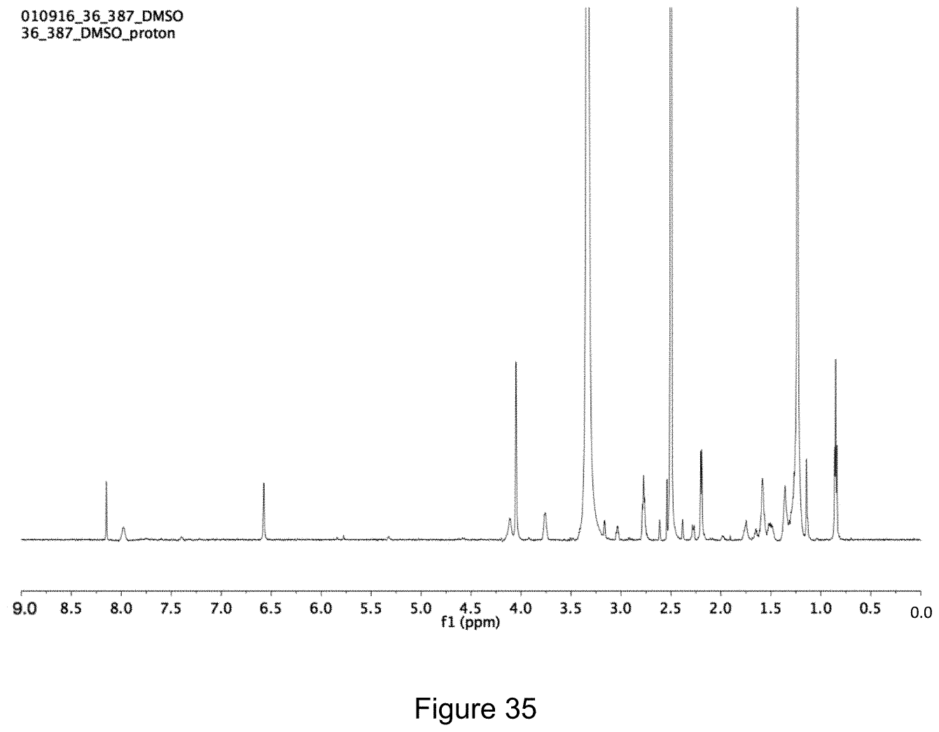

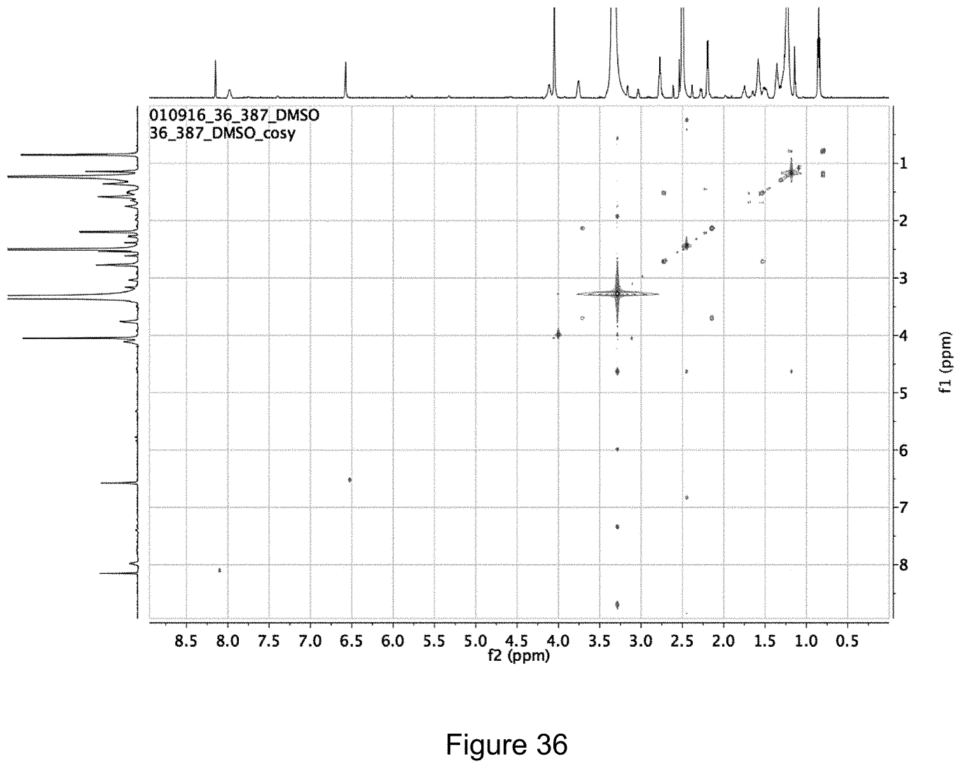

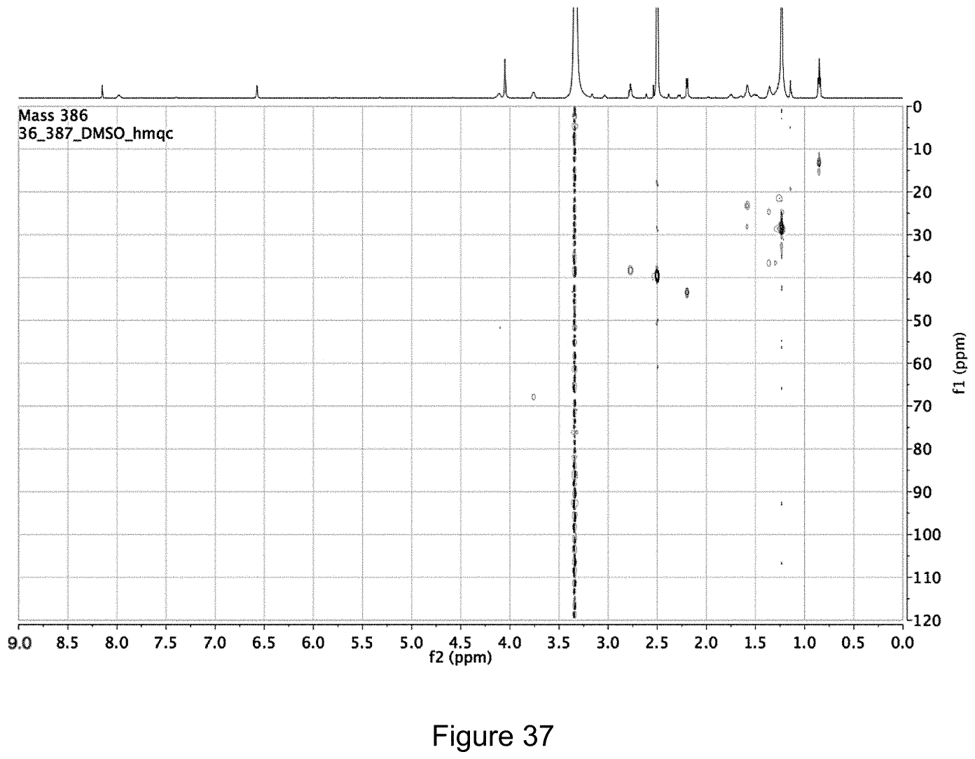

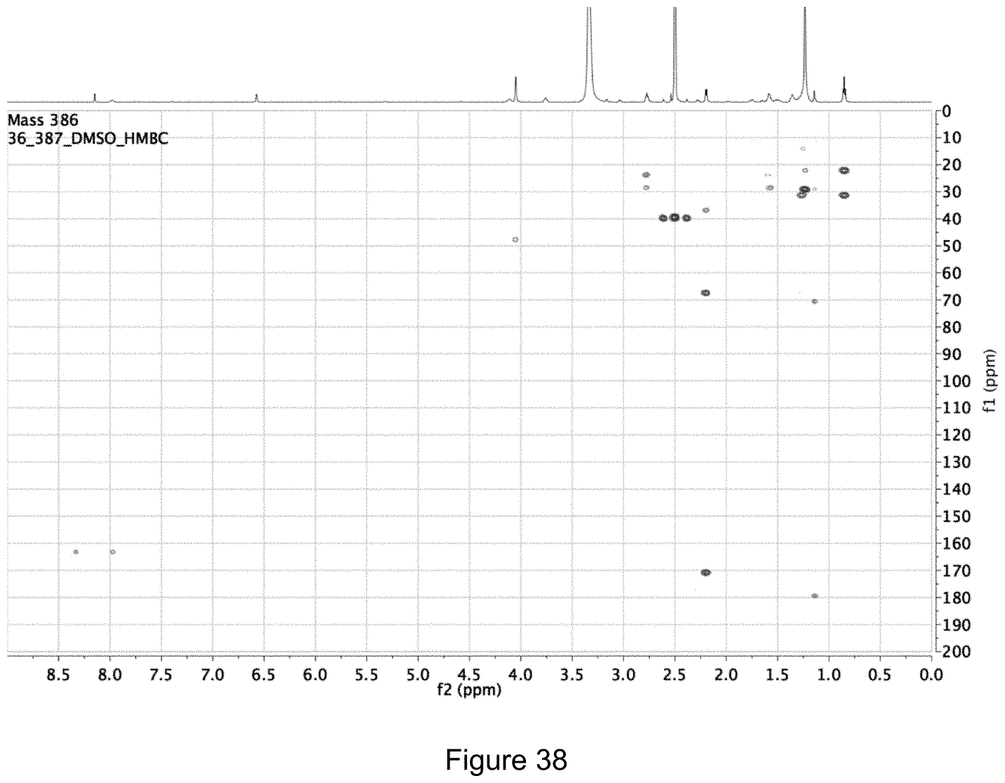

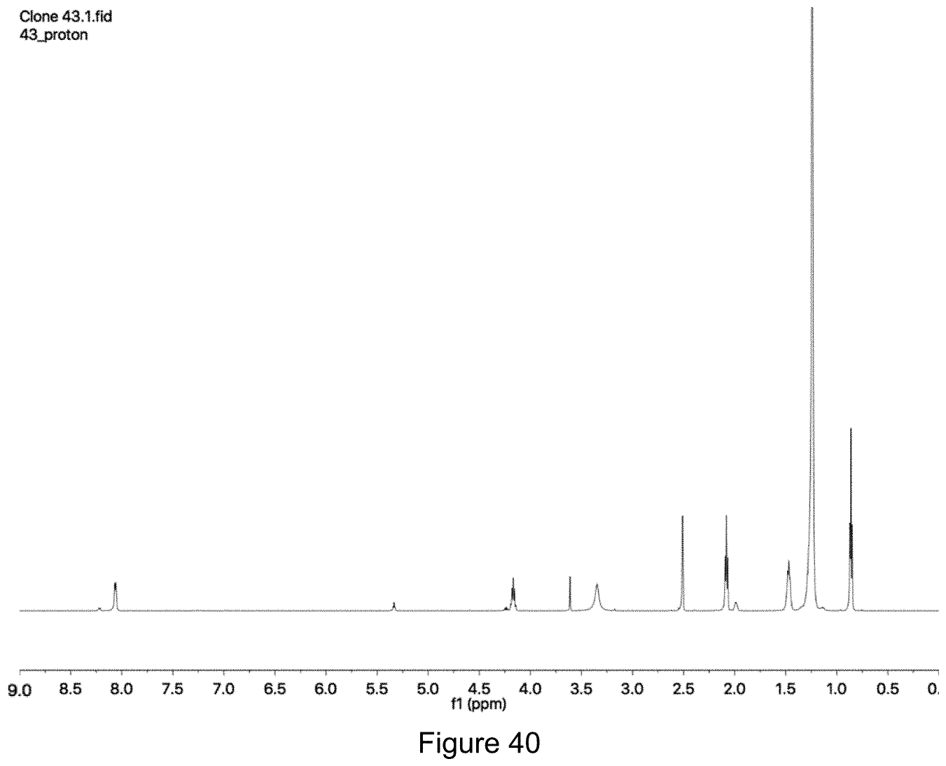

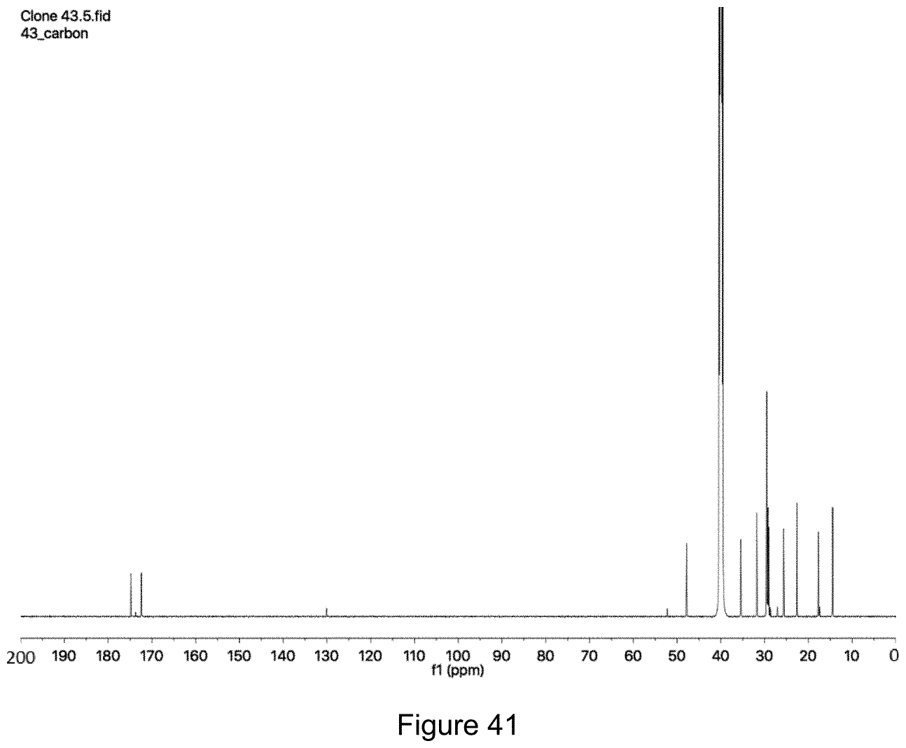

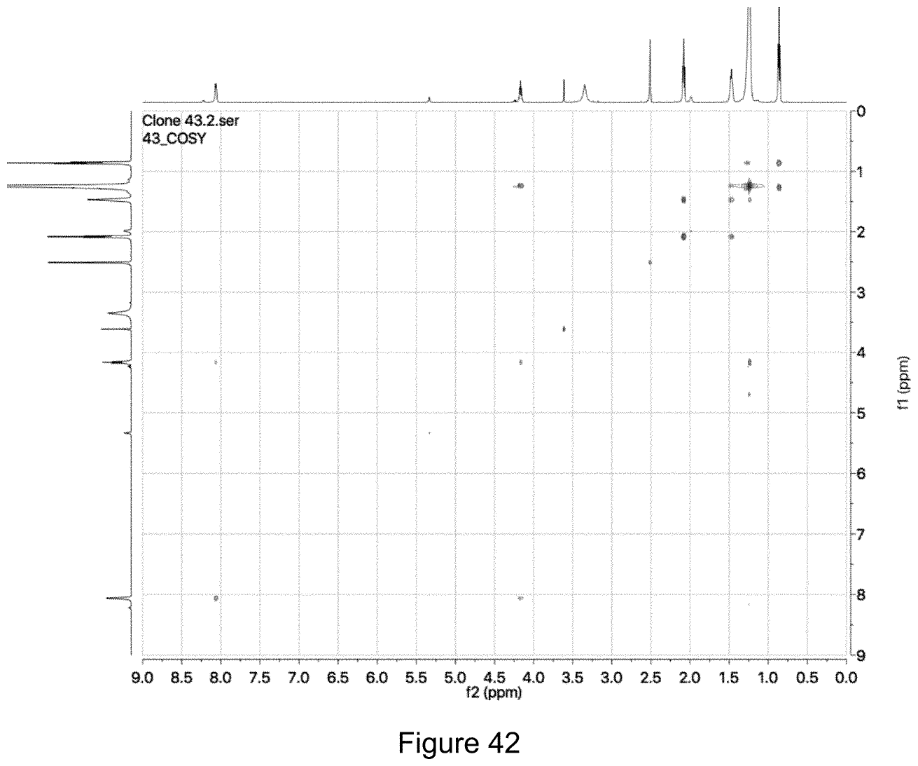

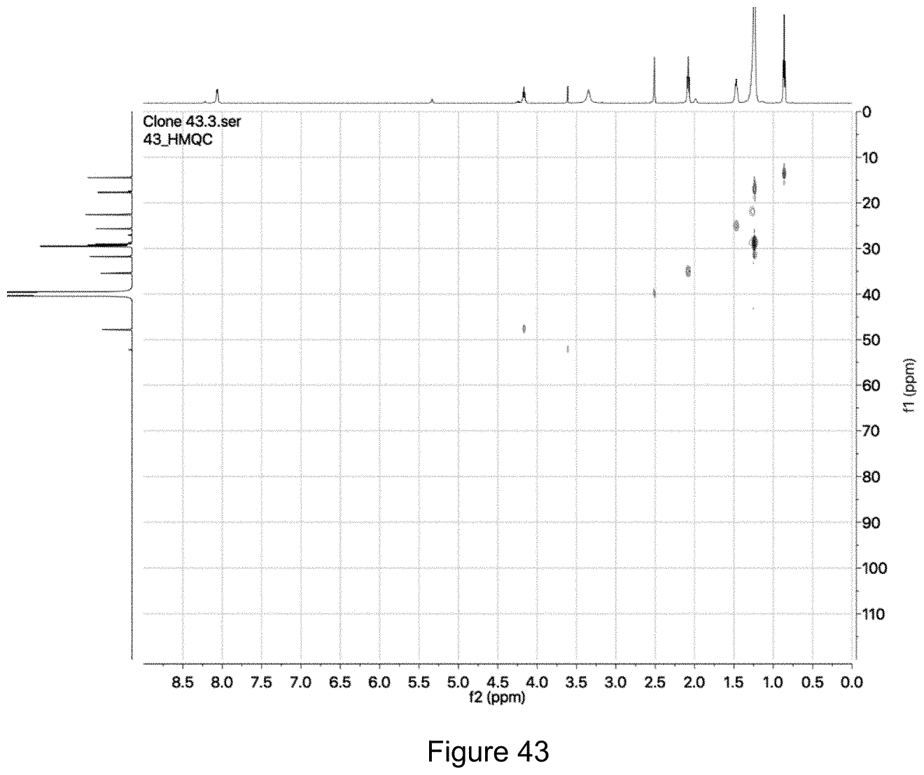

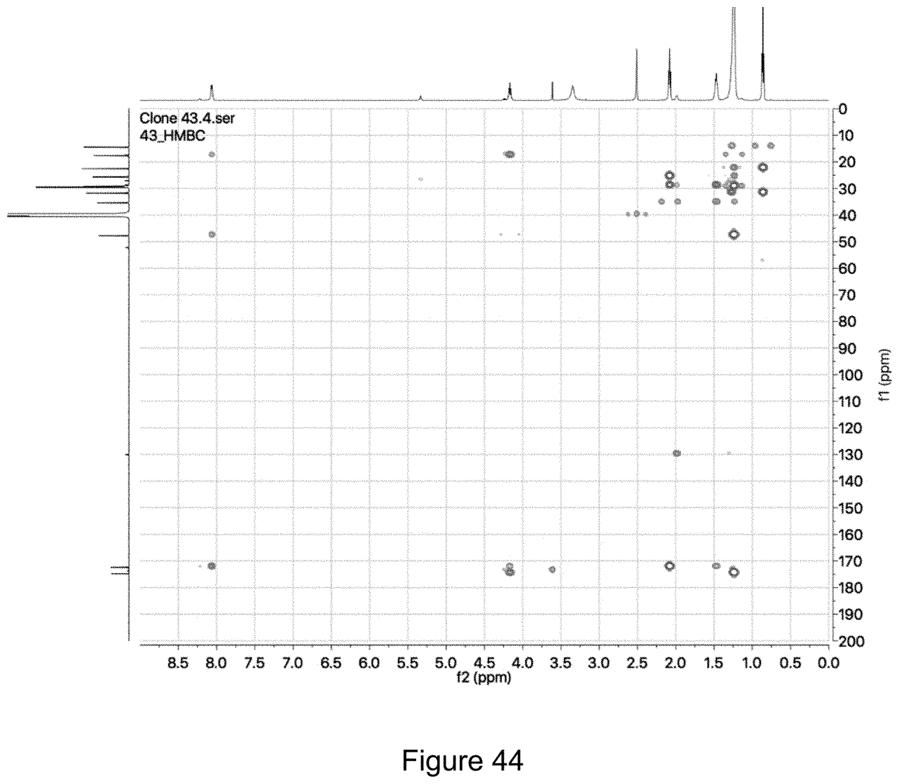

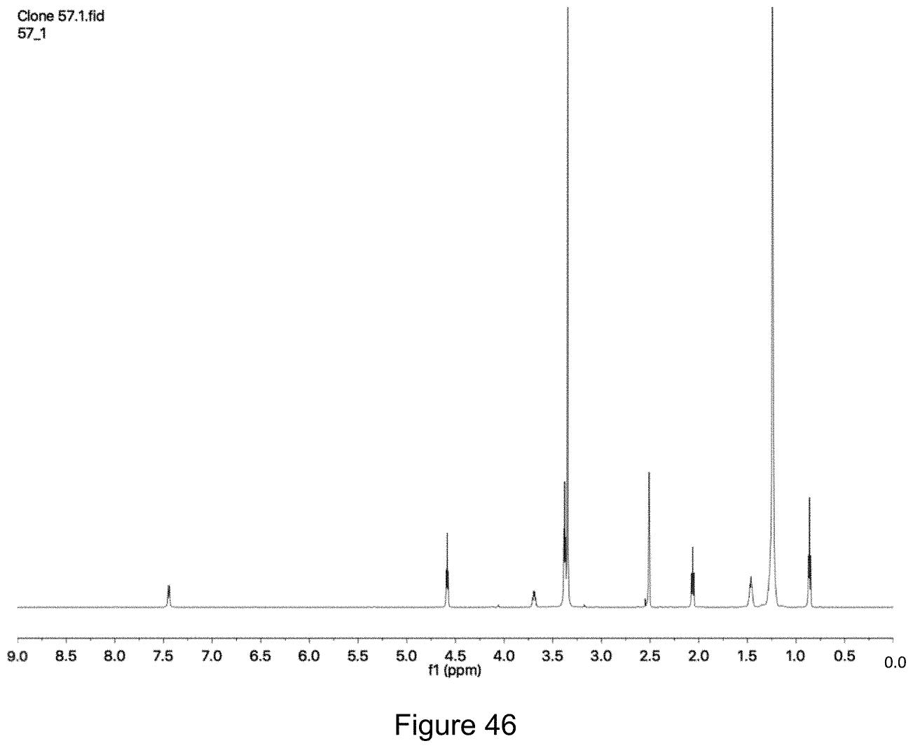

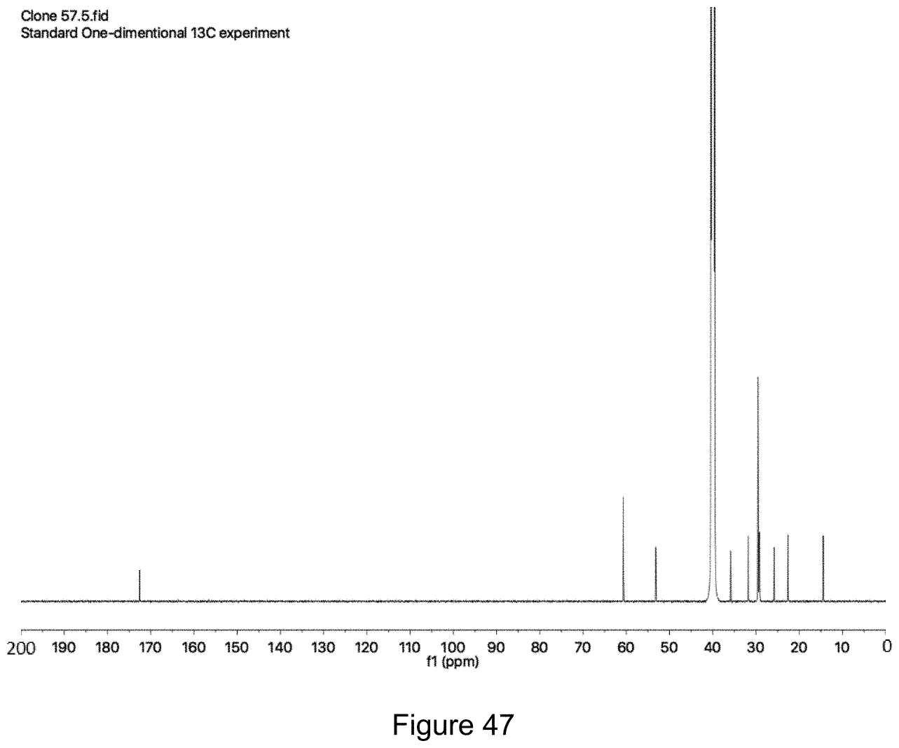

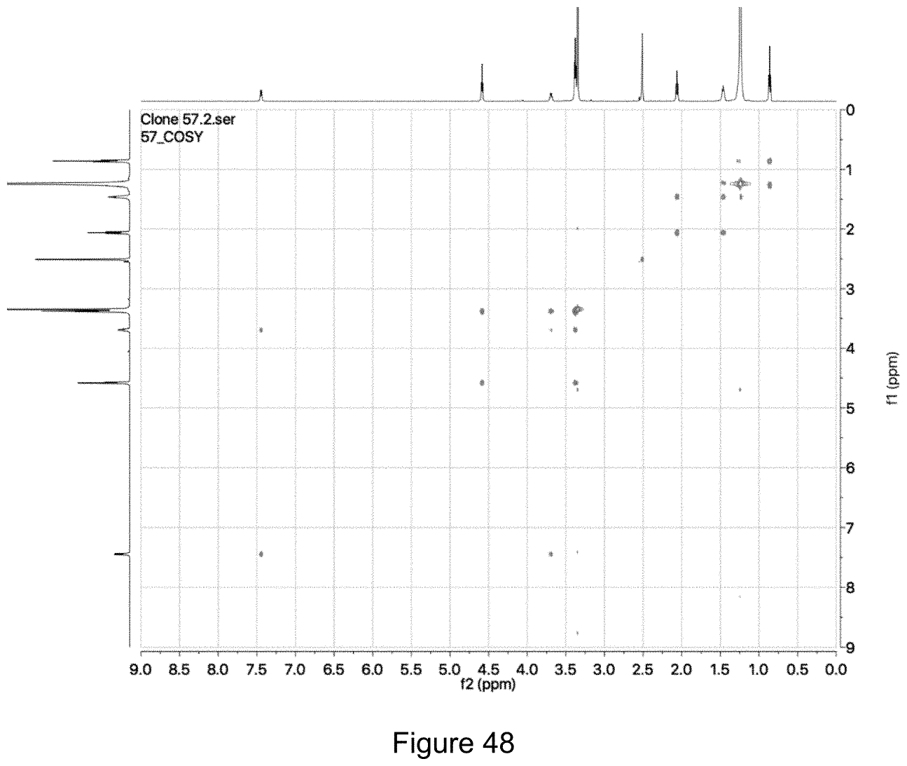

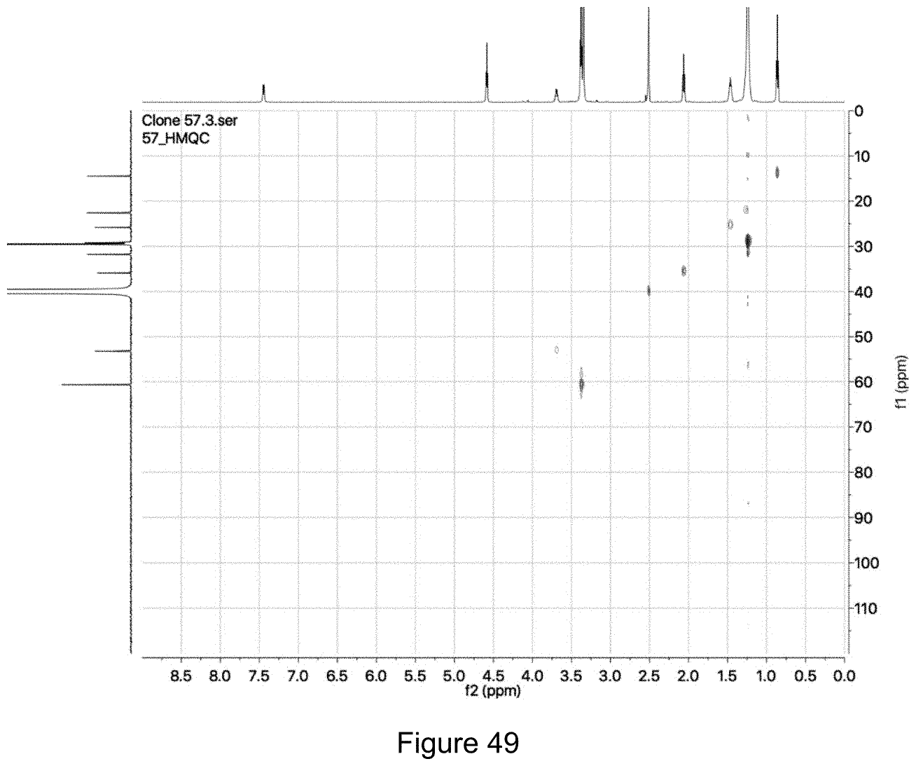

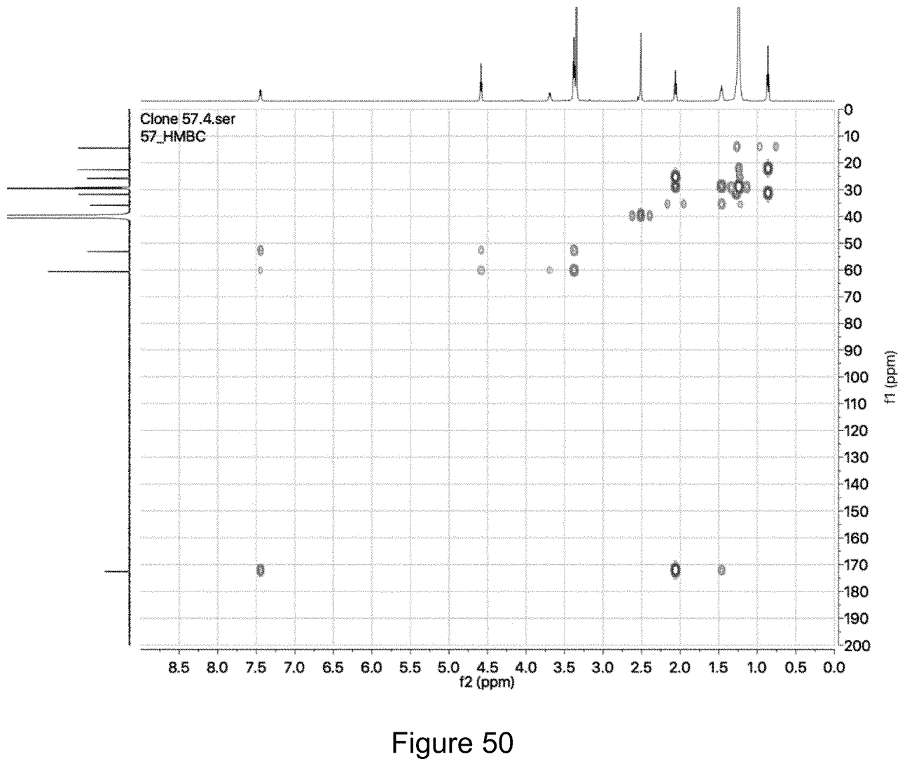

For each group of clones that, based on LCMS analysis, was predicted to produce a different N-acyl amide family one representative clone was chosen for use in molecule isolation studies. Each representative clone was grown as 1.5 L LB cultures in 2.7 L Fernach flasks (30° C., 200 RPM). After 4 days cultures were extracted 2 times with an equal volume of ethyl acetate. Dried ethyl acetate extracts were partitioned by reversed phase flash chromatography (Teledyne Isco, C₁₈ RediSep RF Gold™ 15 g) using the following mobile phase conditions: water:acetonitrile with 0.1% formic acid=10% acetonitrile isocratic for 5 minutes, gradient to 100% acetonitrile over 20 minutes (30 ml/minute). Fractions containing clone specific metabolites were pooled and semi preparative reversed phase HPLC was used to separate individual N-acyl amide molecules. The structures of compounds 2-6 were determined using combination of HRMS, ¹H, ¹³C, and 2D NMR (FIGS. 15-50). Compound 1 was described previously (Cohen et al., 2015, PNAS 112:E4825-34).

hm-NAS Origin Bacterial Species Culture Broth Analysis

Capnocytophaga ochracea F0287 (molecule 2), Klebsiella pneumoniae WGLW1-5 (molecule 3), Neisseria flavescens SKI 14 (molecule 4), and Gemella haemolysans M341 (molecule 6) were obtained from the Biodefense and Emerging Infections Research Resources Repository (BEI Resources) HMP catalogue. Molecule 1 was previously identified in culture broth extracts from cultures of Bacteroides vulgatus.³ Each chosen bacteria contains the identical hm-NAS gene that was heterologously expressed to produce the molecule 2, 3, 4 or 6. Strains were inoculated under sterile conditions into 2 L of LYBHI medium [brain-heart infusion medium supplemented with 0.5% yeast extract (Difco), 5 mg/L hemin (Sigma), 1 mg/ml cellobiose (Sigma), 1 mg/ml maltose (Sigma), 0.5 mg/ml cysteine (Sigma)] and grown anaerobically (C. ochracea) or aerobically (N. flavescens, G. haemolysans, K. pneumonia) for 7 days. Culture broths were extracted with an equal volume of ethyl acetate. To look for the presence of N-acyl amides these extracts were examined by HPLC-MS as was done in heterologous expression experiments. With the exception of family 3, the N-acyl metabolite that was heterologously expressed could be identified in the culture broth extracts from the bacteria that harbored that hm-NAS gene (FIG. 6).

GPCR Screen of N-acyl Amide Small Molecules

For each of the 6 N-acyl amide families (1-6) the analogue produced at the highest level in the heterologous expression experiments was assayed for GPCR activity. In the case of family 4 the major lysine analogue (N-3-hydroxyoleoyl lysine) was screened. Using β-arrestin cell-based assays at 10 uM ligand concentration, agonist and antagonist activity was assessed by DiscoveRx against 168 GPCRs with known ligands as well as 72 orphan GPCRs. The most potent interactions between N-acyl amides and GPCRs were validated by repeating the assay in duplicate and generating dose response curves. Synthetic N-acyl amides were assayed in the same fashion.

### Synthesis of Proteinogenic Amino Acid Containing N-acyl-palmitoyl Analogues

Wang resins with preloaded amino acids were purchased from Matrix Innovation. Coupling reagents (PyBOP and C1-HOBt) were purchased from P3 BioSystems. Palmitoyl chloride and all other reagents were purchased from Sigma-Aldrich. Dimethylformamide (DMF) was added to preloaded Wang resins (˜80 mg) and incubated for 30 minutes. Removal of N-Fmoc from swollen resins was accomplished by two rounds of piperidine treatment [20% solution in DMF (v/v), 3 ml] for 3 and 10 minutes, followed by several washes with DMF. Palmitoyl chloride (1 equivalent) in DMF was then added and the resin suspension was shaken for 2 hours at room temperature. The N-acylated amino acid product was cleaved from the resins by treatment with trifluoroacetic acid (TFA) supplemented with 2.5% (v/v) water and 2.5% (v/v) triisopropylsilane (TIPS). After evaporation of TFA the crude product was purified by automated reversed phase flash chromatography (Teledyne Isco system, C₁₈ RediSep RF Gold™ 15 g), binary solvent system: water and acetonitrile supplemented with 0.1% acetic acid. All final products were verified by MS (Table 5).

TABLE 5
Synthetic N-acyl amino acid MS data (observed m/z
in positive ion mode).

| Code | Amine moiety | MW | Obs m/z |
|---|---|---|---|
| A | Alanine | 327.3 | 328.35 |
| R | Arginine | 412.3 | 413.49 |
| N | Asparagine | 370.3 | 371.40 |
| D | Aspartatic acid | 371.3 | 372.40 |
| C | Cysteine | 359.2 | 360.37 |
| Q | Glutamine | 384.3 | 385.44 |
| E | Glutamic acid | 385.3 | 386.43 |
| G | Glycine | 313.3 | 314.34 |
| H | Histidine | 393.3 | 394.44 |
| I | Isoleucine | 369.3 | 369.23 |
| L | Leucine | 369.3 | 369.24 |
| K | Lysine | 384.3 | 385.47 |
| M | Methionine | 387.3 | 388.42 |
| F | Phenylalanine | 403.3 | 404.45 |
| P | Proline | 353.3 | 354.42 |
| S | Serine | 343.3 | 344.35 |
| T | Threonine | 357.3 | 358.42 |
| W | Tryptophan | 442.3 | 443.48 |
| Y | Tyrosine | 419.3 | 420.48 |
| V | Valine | 355.3 | 356.48 |

In-Vitro Study of GLP-1 Release from GLUTag Cells

Oleoyl ethanolamide and 2-oleoyl glycerol were purchased from Cayman Chemical Company and resuspended in DMSO to a concentration of 10 mM. N-oleoyl serinol was isolated and purified in the same manner as N-palmitoyl serinol described above and confirmed by ¹H NMR and HRMS. N-oleoyl serinol was resuspended at 10 mM concentration in DMSO. GLUTag cells were obtained from the Mangelsdorf Lab (University of Texas Southwestern) with permission from Daniel Drucker (Mount Sinai Hospital Toronto). GLUTag cells were grown in DMEM, low glucose, GlutaMAX (ThermoFisher) supplemented with 10% FBS and 1% Penicillin/Streptomycin. Once cells grew to 80% confluence they were harvested and plated 1:1 into 24 well culture plates in fresh culture media at 50,000 cells per well. After overnight growth in culture plates, cells were washed twice with Krebs buffer supplemented with 20 μL per ml of DPP4 inhibitor (Millipore). GLUTag cells were incubated for 30 minutes in supplemented Krebs buffer and compounds were added at 1 μM and 100 μM. Cells were incubated with compounds for 2 hours. Media was then collected, centrifuged at 500×g (4° C.) for 5 minutes and cell free supernatant was analyzed for GLP-1 level using the Active GLP-1 V2 kit (Mesoscale Discovery). Experiments were performed in duplicate and data from both experiments were pooled for FIG. 5C (N=6 for OEA, N-oleoyl serinol and DMSO and N=4 for 2-OG).

Colonization of Germ-Free Mice with N-acyl Serinols Producing E. coli

C57BL/6 mice were maintained in sterile isolators with autoclaved food and water. 8-week-old mice were used for all experiments. For colonization studies 5 ml of an overnight culture (LB with 50 μg/ml kanamycin) of E. coli transformed with pET28c:hm-NAS N-acyl serinol synthase (treatment group) or E. coli transformed with the empty pET28c vector (control group) was centrifuged at 500×g for 2 minutes, the supernatant was decanted and the cells were resuspended in 2 ml of sterile PBS. Mice were gavaged with 100 μL of bacterial culture immediately upon export from sterile isolators. After colonization mice were housed in specific-pathogen-free conditions and fed with autoclaved water and food. Water was supplemented with 35 μg/ml kanamycin and 25 mM IPTG (Mimee et al., 2015, Cell Syst 1:62-71). Fecal pellets from mice were analyzed each week for 3 weeks to confirm colonization by the appropriate bacteria and to check for contamination by plating on LB agar with and without kanamycin 50 μg/ml. Plasmids were isolated and restriction mapped from these colonies to confirm the presence of the correct hm-NAS gene insert or lack thereof. Ethyl acetate extracts from broth cultures were also examined as previously discussed to confirm the production of N-acyl serinols in the treatment group. In the first experiment 7 mice were studied [3 (1M, 2F) in the treatment group and 4 (2M, 2F) in the control group]. The replicate experiment also consisted of 7 mice (all female, 3 in the treatment group and 4 in the control group). Mice were all individually caged and at the end of each week food consumption and weight were measured. The animal experiments were not randomized and the investigators were not blinded to the allocation during experiments and outcome assessment. No statistical methods were used to predetermine sample size.

Generation of Active Site pET28c:hm-NAS N-acyl Serinol Synthase Mutant

Conserved active site residues in bacterial NASs were identified in previous biochemical and X-ray crystalography studies.⁴² To create a catalytically inactive N-acyl serinol synthase we changed a key glutamic acid residue (Glu91) to alanine. The point mutation was created by PCR using pET28c:hm-NAS N-acyl serinol synthase vector as template and the following primers F-GTTCTGTGCGATACGTCTCC (SEQ ID NO: 1) and R-GCCTTTCACAGGCAGATATTC (SEQ ID NO:2). The position of point mutation is underlined in the F primer. The resulting PCR reaction was digested with DpnI to remove any remaining methylated vector. The PCR product was then phosphorylated, column purified and blunt end ligated (End-It, Epicentre). The vector was transformed into EC100:DE3 cells and the point mutation was confirmed by Sanger sequencing. When transformed into E. coli, the resultant E94A mutant construct did not confer the production of any detectable N-acyl serinols. Cultures were grown under the same conditions as the original N-acyl serinol synthase producing clone (FIG. 13).

Oral Glucose Tolerance Test

One week post colonization mice were fasted overnight (16 hours) and then administered a 2 g/kg oral glucose tolerance test (40% glucose solution). Blood glucose was measured by tail bleed at time 0 prior to the glucose gavage, then 15, 30, 60, 90 and 120 minutes post gavage. Glucose was measured by tail bleed (Breeze 2 Bayer). After one week the IPTG was removed from the mouse drinking water and mice were allowed to equilibrate for an additional 2 weeks (Mimee et al., 2015, Cell Syst 1:62-71). Three weeks post colonization the oral glucose tolerance test was repeated. Blood glucose levels at each time point during the glucose tolerance tests were compared between groups using a Students T-test with significance threshold of p<0.05.

Insulin and GLP-1 Measurement

Mice were given an OGTT as previously described. At 15 minutes, blood was collected by submandibular bleed and immediately mixed with 10 μL of 0.5M EDTA and 5 μL of DPPIV inhibitor (Millipore, DPP4-010) per 500 μL of blood. Treated blood was spun at 2,000×g for 15 minutes at 4° C. Plasma was collected and immediately placed at −80° C. Insulin was measured using the Crystal Chem Ultra Sensitive Mouse ELISA kit and active GLP-1 was measured using the Mesoscale Discovery Active GLP-1 V2 kit. Samples were analyzed for insulin in triplicate and GLP-1 in duplicate. Insulin was measured from mice in one experiment (N=6 mice in each group). GLP-1 was measured from mice in two independent experiments (N=9 mice in control, N=10 mice in treatment). All mice were male and of the same size distribution.

### N-acyl Serinol Metabolite Measurement in Mouse Cecal Samples and Human Stool

After withholding IPTG for two weeks, mice from the first experimental set were re-exposed to IPTG in the drinking water for 1 week to induce hm-NAS gene expression and N-acyl serinol production. Mice were sacrificed and cecal samples taken. Fresh cecal stool from two control mice and two treated mice was resuspended in 5 ml of sterile PBS and extracted 1:1 with ethyl acetate. Crude extracts were dried in vacuo and resuspended in methanol normalized by crude extract weight. Each extract was then analyzed by reversed phase liquid chromatography coupled to a 6550 Q-TOF mass spectrometer. Peak identities were confirmed by accurate mass, and also by comparison of chromatographic retention time and MS/MS spectra to those of the purified N-palmitoyl serinol standard. In both mice in the treatment group N-palmitoyl serinol could be detected in the cecal samples whereas N-palmitoyl serinol was detected in neither of the mice in the control group (FIG. 11). Stool samples were collected from human subjects prior to bone marrow transplant. Fresh stool samples were processed in the same manner as the mouse cecal samples described above.

Compound Isolation and NMR Structure Determination Overview

For each compound family, dried ethyl acetate extracts were partitioned by reversed phase flash column chromatography (Teledyne Isco, C₁₈ RediSep RF Gold™15 g) using the following mobile phase conditions: solvent A:B (water:acetonitrile with 0.1% formic acid) 10% B isocratic for 5 min, gradient to 100% B over 20 minutes (30 ml/min). Fractions containing clone specific metabolites as identified by LCMS were pooled and semi-preparative reversed phase HPLC was used to separate individual N-acyl amide molecules (Waters XBridge™ C18, 10 mm×250 mm: 4.8 ml/min: solvent A:B, water:acetonitrile with 0.1% TFA). Chromatographic details for each metabolite analyzed by NMR.

Molecule 2: retention time 8.5 min, gradient 85% B to 100% B over 20 min

Molecule 3: retention time 13 min, isocratic 70% B

Molecule 4a: retention time 17 min, isocratic 40% B for 5 min, gradient from 40% to 72% B over 15 min

Molecule 4b: retention time 16 min, isocratic 40% B for 5 min, gradient from 40% to 72% B over 15 min

Molecule 5: retention time 9.5 min, isocratic 60% B for 5 min, gradient from 60% to 100% B over 10 min

Molecule 6: retention time 17 min, isocratic 50% B for 5 min, gradient from 50% to 100% B over 15 min

Each E. coli strain transformed with a single hm-NAS gene produced a family of related N-acyl amides. With the exception of compound family 4 (compounds 4a and 4b), ¹H NMR and MS analysis indicated that cultures produced metabolites with the same amine head group but different acyl substituents. Based on MS data acyl substituents were predicted to be fully saturated or mono-unsaturated and differ only slightly in length. The most common acyl chains incorporated by hm-NASs are from 14-18 carbons in length. These can be modified by β-hydroxylation or a single unsaturation. In the case of family 4 ¹H NMR data suggested two different amine head groups. The major N-acyl amide produced by weight for each family was selected for in depth structural analysis. In the case of family 4, the major N-acyl amide by weight for each head group was structurally characterized (compounds 4a and 4b).

Family 2—Compound 2

For family 2 complete separation individual N-acyl amides from each other was not achieved. The material we structurally characterized contained a mixture of two N-acyl amides (one major and one minor metabolite) that differed by 26 units (m/z: [M+H]⁺ 611, 637). The HRMS predicted molecular formula of the dominant compound in the mixture was C₃₆H₇₀N₂O₅ (m/z: [M+H]⁺Calcd C₃₆H₇₁N₂O₅ 611.5363; found 611.5385). The COSY spectrum defined 5 spin systems. The 5-carbon-NH COSY spin system together with an HMBC correlation between H-1′(δ_{H} 3.03) and C-2′ (δ_{C} 176.8) supported the presence of an N-acylated lysine substructure. The 4-carbon COSY spin system together with an HMBC correlation from H-2 to the C-1 carbonyl indicated the presence of a C-3 oxidized fatty acid. The presence of two separate acyl chains was suggested by the presence of two terminal methyl triplets in the ¹H NMR spectrum. The appearance of the mass for N-hydroxypalmitoyl lysine in the HR MS/MS fragmentation analysis allowed us to define the structure of one acyl substituent as [C16:3-OH]. The length of the second acyl group was predicted based on the predicted molecular formula [C14]. Based on this analysis the final structure of compound 2 was determined to be 3-(myristoyloxy)palmitoyl lysine. Based on the small olefinic proton signal (δ_{H} 5.32) in the ¹H NMR spectrum and MS/MS fragmentation analysis the minor compound in the mixture (m/z: [M+H]⁺ 637) was predicted to contain a double bond and longer acyl substituents (FIGS. 15-20).

Family 3—Compound 3

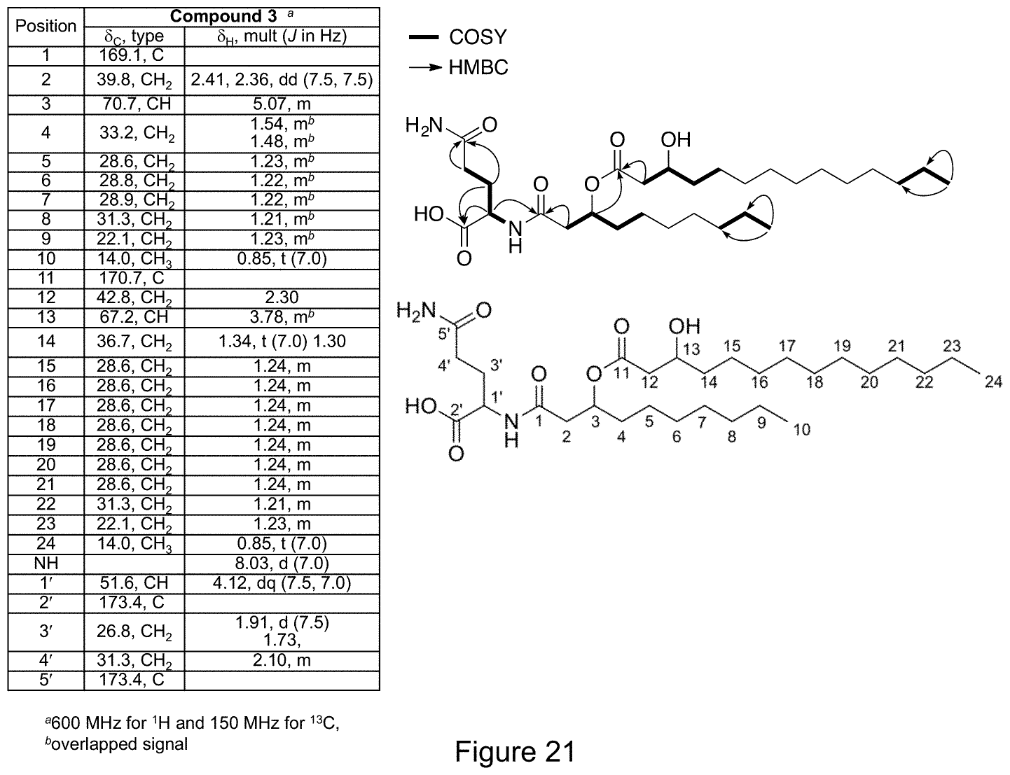

The molecular formula predicted by HRMS for compound 3 was C₂₉H₅₄N₂O₇ (m/z: [M+H]⁺Calcd C₂₉H₅₅N₂O₇ 543.4009, found 543.4009). The ¹H NMR of compound 3 exhibited two oxygenated methines, a group of highly overlapped aliphatic methylene proton signals (δ_{H} 1.24-1.21) and two terminal methyl triplets. The ¹³C NMR of compound 3 exhibited four carbonyl carbons, two oxygen bearing carbons, an aliphatic methine carbon, two methyl carbons, ten distinguished aliphatic methylene carbons and additional overlapping aliphatic methylene carbons (δ_{C} 28.8-28.6). COSY correlations defined five spin systems. Starting from the 3-carbon-NH COSY spin system, HMBC correlations from H-1′ (δ_{H} 4.12) and H₂-3′ (δ_{H} 1.91 and 1.73) to C-2′ (δ_{C} 173.4) and H-4′ (δ_{H} 2.10) and H₂-3′ (δ_{H} 1.91 and 1.73) to C-5′ (δ_{C} 173.4) defined the structure of glutamine. An HMBC correlation from H-3 (δ_{H} 5.07) to C-11 (δ_{C} 170.7) was used to connect the glutamine through an amide bond to a C-3 oxidized fatty acid. HMBC correlations from H-1′ (δ_{H} 4.12) and H₂-12 to C-11 allowed us to connect a second fatty acid to this substructure through an ester bond. The exact nature of each fatty acid was defined by HRESI-MS/MS fragmentation analysis. Based on this analysis the N-acyl fatty acid chain was predicted to contain 10 carbons and the acyl fatty chain was predicted to contain 14 carbons. Thus, the structure of 3 was determined to be 3-[(3-OH-myristoyl)oxy]decanoyl glutamine (FIGS. 21-27).

Family 4—Compound 4a

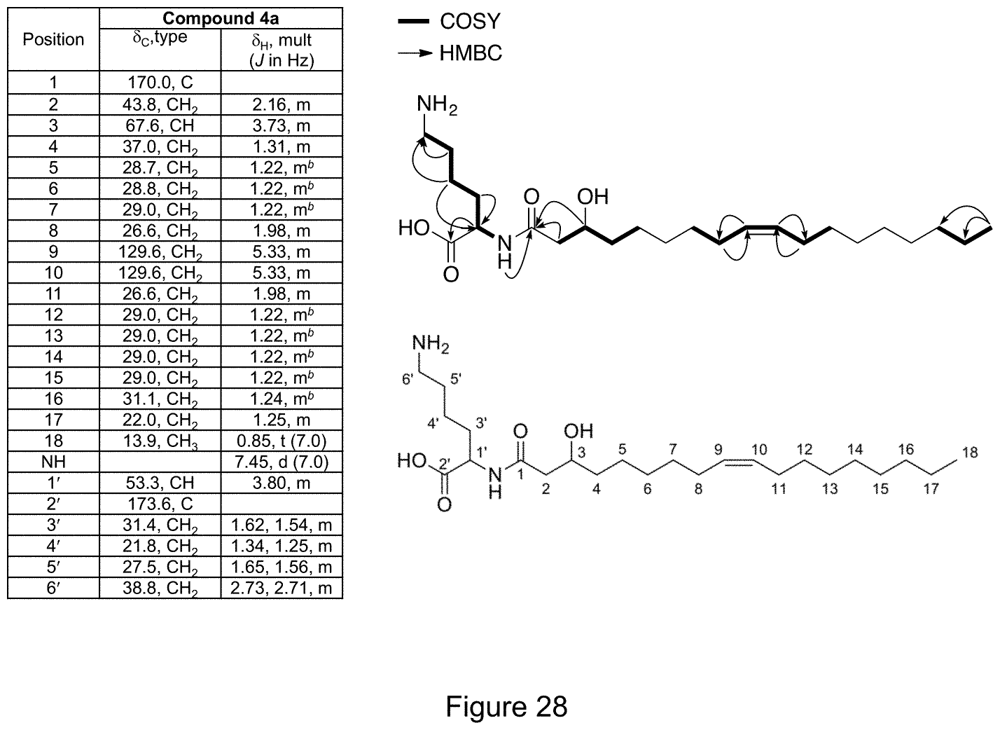

The major metabolite in family 4 (4a) was predicted by HRMS to have the following molecular formula: C₂₄H₄₆N₂O₄ (m/z: [M+H]⁺Calcd C₂₄H₄₇N₂O₄ 427.3536, found 427.3531). Through analysis of ¹H and edited HSQC spectra two olefinic protons, an oxymethine proton, a deshielded aliphathic methine proton, a terminal methyl triplet proton and a group of overlapping aliphatic methylene (δ_{H} 1.24˜1.22) were revealed. From the COSY spectrum four spin systems were established. The 5 carbon spin system with COSY correlations from H-1′ to H₂-6′ along with empirical carbon and proton chemical shift data and HMBC correlations from H-1′ (δ_{H} 3.80) to C-2′ (δ_{C} 173.6) led to the construction of lysine. HMBC correlations from H₂-2 to C-1 and from NH to C-1 of HMBC indicated that the lysine as connected to a C-3 hydroxylated fatty acid moiety through an amide bond. Based on the predicted molecular formula for compound 4a the fatty acid side chain was determined to be [C18:1]. The position of the double bond is predicted based on the position that is most frequently seen in E. coli lipids and has been seen in other N-acyl amino acids heterologously produced in E. coli.¹ Thus, the structure of 4a was determined to be N-3-OH-oleoyl lysine (FIGS. 28-33).

Family 4—Compound 4b

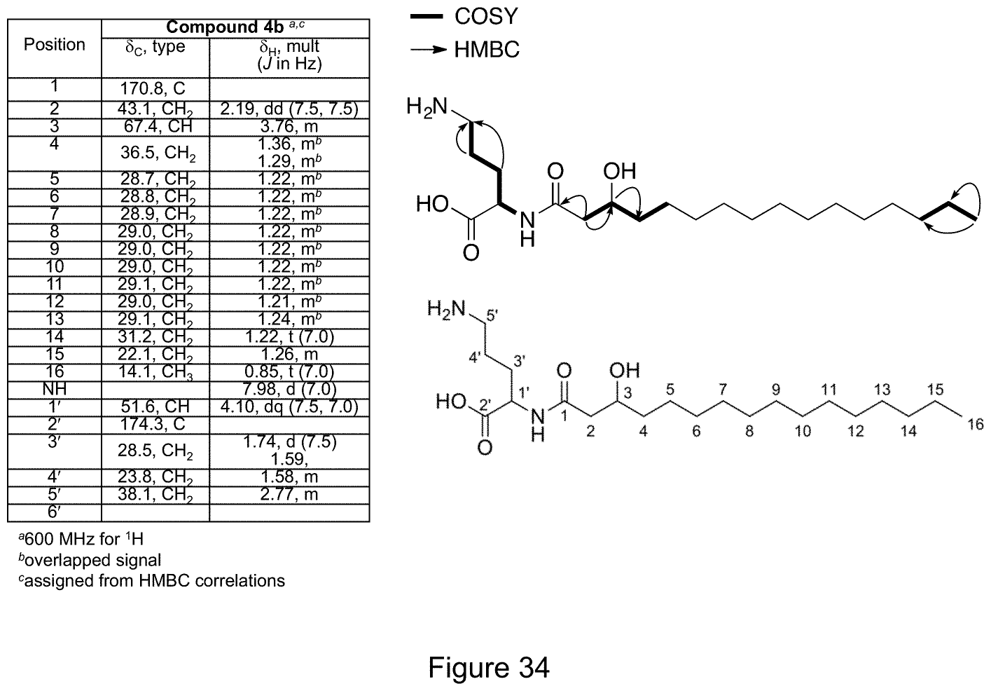

The second major N-acyl amide from family 4 by weight had an HRMS predicted molecular formula of C₂₁H₄₂N₂O₄ (compound 4b: m/z: [M+H]⁺Calcd C₂₁H₄₃N₂O₄ 387.3223, found 387.3226). NMR spectra for 4b were nearly identical to those collected for 4a with the exception of 1) the disappearance of the olefinic protons in the ¹H NMR and 2) the replacement of the 5-carbon-NH COSY spin system with a 4-carbon-NH COSY spin system. Based on these differences and the HRMS predicted molecular formula for 4b the structure of 4b was determined to be N-3-OH-palmitoyl ornithine (FIG. 34-38).

Family 5—Compound 5

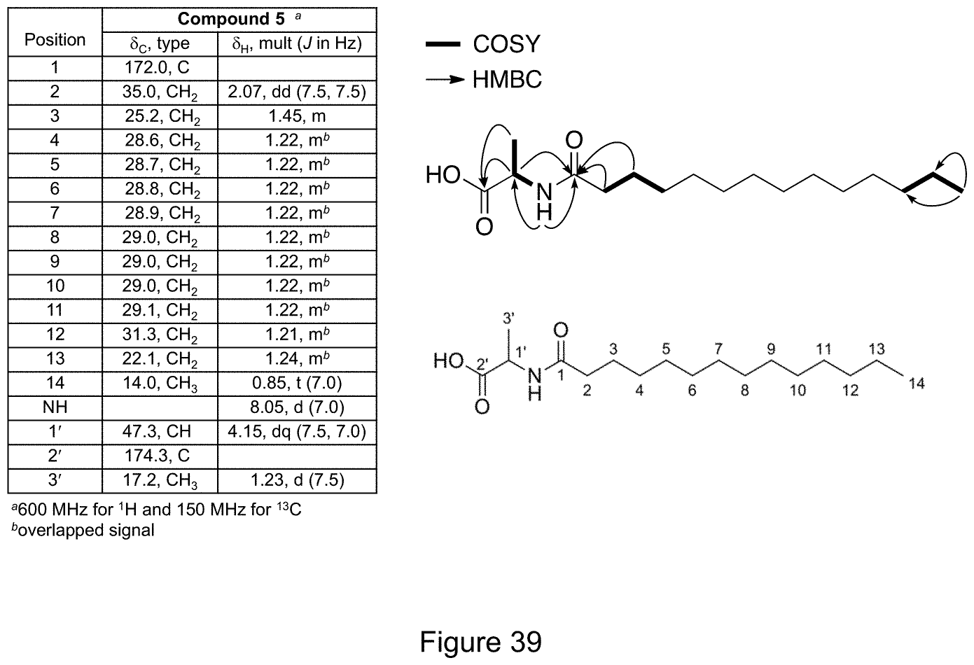

The HRMS predicted molecular formula for compound 5 was C₁₇H₃₃NO₃ (m/z: [M+Na]⁺Calcd C₁₇H₃₃NO₃Na 322.2358, found 322.2356). The ¹H NMR spectrum of 5 exhibited chemical shifts characteristic of a saturated fatty acid, a deshielded methine, 2 methyls (doublet and triplet) and a deshielded NH. The ¹³C NMR spectrum of 5 exhibited two clear carbonyl carbons and one predicted N-substituted carbon. Three spin systems were observed in the COSY spectrum. HMBC correlations from the methyl doublet of a 2-carbon COSY spin system to the carbonyl carbon (C-2′) indicated the presence of an alanine in 5. Empirical ¹³C NMR chemical shift data and HMBC correlations from H₂-2 to C-1 and the NH proton to C-1 and C-1′ indicated that the fully saturated fatty acid was connected to the alanine through an amide bond. Based on the HRMS predicted molecular formula for 5 the length of a fatty acid was determined to be C14. Thus, the structure of 5 was determined to be N-myristoyl alanine (FIGS. 39-44).

Family 6—Compound 6

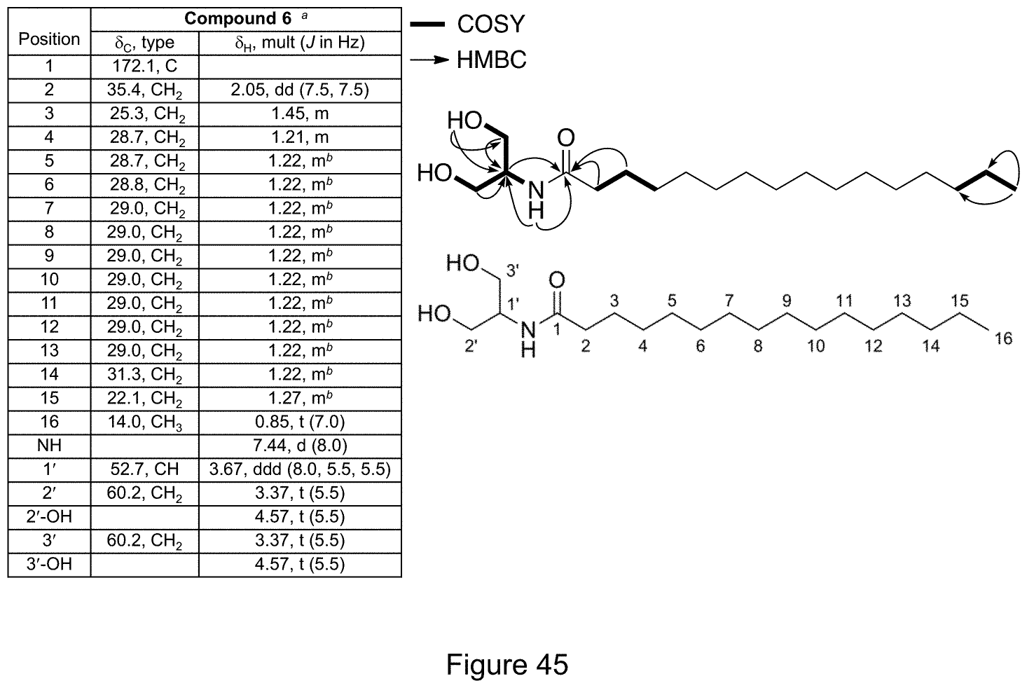

The HRMS predicted molecular formula for compound 6 was C₁₉H₃₉NO₃ (m/z: [M+H]⁺Calcd C₁₉H₄₀NO₃ 330.3008, found 330.3014). Analysis of the ¹H NMR spectrum indicated the presence of a saturated fatty acid moiety [e.g., overlapping aliphatic methylene proton signals (δ_{H} 1.23˜1.21) and a terminal methyl triplet proton triplet (δ_{H} 0.85)]. In the ¹³C and HMQC NMR spectra of 6 we observed one carbonyl carbon, two equivalent oxymethylene carbons (C-2′ and C-3′) and a deshielded aliphatic methine carbon. Analysis of the COSY spectrum identified three spin systems. Extensive COSY and HHMC (see figure below) correlations established a serinol substructure. Based on HMBC correlations from NH to C-1 and C-1′ and weak long-range HMBC correlations from H-1′ to C-1 the serinol was connected to the fatty acid through an amide bond. The length of a fatty acid was determined as C16 based on the predicted molecular formula. Thus, the structure of 6 was determined to be N-palmitoyl serinol (FIGS. 45-50).

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A genetically engineered cell, wherein the cell expresses a human microbial N-acyl synthase (hm-NAS) gene.

2. The cell of claim 1, wherein the cell is a non-pathogenic bacterial cell.

3. The cell of claim 1, wherein the cell is capable of producing a N-acyl amide.

4. The cell of claim 1, wherein the hm-NAS gene is selected from a hm-NAS gene of table 1 or table 2.

5. The cell of claim 4, wherein the hm-NAS gene is N-acyl serinol synthase.

6. A probiotic composition comprising the cell of claim 1.

7. The probiotic composition of claim 6, wherein the composition further comprises a prebiotic.

8. A method for modulating a G protein-coupled receptor (GPCR) activity in a subject, the method comprising administering to the subject an effective amount of a composition comprising at least one selected from the group consisting of a genetically engineered cell, an hm-NAS gene, and a N-acyl amide, wherein the engineered cell expresses a human microbial N-acyl synthase (hm-NAS) gene.

9. The method of claim 8, wherein the hm-NAS gene is selected from a hm-NAS gene of table 1 or table 2.

10. The method of claim 8, wherein the N-acyl amide is represented by Formula (1): 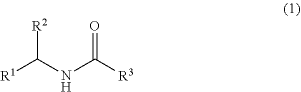
wherein R¹ is selected from the group consisting of carboxylate and CH₂OH;
R² is selected from the group consisting of H, (C₃-C₄)alkyl-NH₃⁺, (C₃-C₄)alkyl-NH₂, C₂ alkyl-C(═O)NH₂, CH₂OH, and methyl; and
R³ is selected from the group consisting of (C₉-C₁₈)alkyl, (C₉-C₁₈)alkenyl, wherein the (C₉-C₁₈)alkyl and (C₉-C₁₈)alkenyl are optionally substituted.

12. The method of claim 8, wherein the GPCR is enriched in the gastrointestinal mucosa.

13. The method of claim 8, wherein the GPCR is selected from the group consisting of ADCYAP1R1, ADORA3, ADRA1B, ADRA2A, ADRA2B, ADRA2C, ADRB1, ADRB2, AGTR1, AGTRL1, AVPR1A, AVPR1B, AVPR2, BAI1, BAI2, BAI3, BDKRB1, BDKRB2, BRS3, C3AR1, C5AR1, C5L2, CALCR, CALCRL-RAMP1, CALCRL-RAMP2, CALCRL-RAMP3, CALCR-RAMP2, CALCR-RAMP3, CCKAR, CCKBR, CCR1, CCR10, CCR2, CCR3, CCR4, CCR5, CCR6, CCR7, CCR8, CCR9, CCRL2, CHRM1, CHRM2, CHRM3, CHRM4, CHRM5, CMKLR1, CNR1, CNR2, CRHR1, CRHR2, CRTH2, CX3CR1, CXCR1, CXCR2, CXCR3, CXCR4, CXCR5, CXCR6, CXCR7, DARC, DRD1, DRD2L, DRD2S, DRD3, DRD4, DRD5, EBI2, EDG1, EDG3, EDG4, EDG5, EDG6, EDG7, EDNRA, EDNRB, F2R, F2RL1, F2RL3, FFAR1, FPR1, FPRL1, FSHR, G2A, GALR1, GALR2, GCGR, GHSR, GHSR1B, GIPR, GLP1R, GLP2R, GPR1, GPR101, GPR103, GPR107, GPR109A, GPR109B, GPR119, GPR12, GPR120, GPR123, GPR132, GPR135, GPR137, GPR139, GPR141, GPR142, GPR143, GPR146, GPR148, GPR149, GPR15, GPR150, GPR151, GPR152, GPR157, GPR161, GPR162, GPR17, GPR171, GPR173, GPR176, GPR18, GPR182, GPR20, GPR23, GPR25, GPR26, GPR27, GPR3, GPR30, GPR31, GPR32, GPR35, GPR37, GPR37L1, GPR39, GPR4, GPR45, GPR50, GPR52, GPR55, GPR6, GPR61, GPR65, GPR75, GPR78, GPR79, GPR83, GPR84, GPR85, GPR88, GPR91, GPR92, GPR97, GRPR, HCRTR1, HCRTR2, HRH1, HRH2, HRH3, HRH4, HTR1A, HTR1B, HTR1E, HTR1F, HTR2A, HTR2C, HTR5A, KISS1R, LGR4, LGR5, LGR6, LHCGR, LTB4R, MC1R, MC3R, MC4R, MC5R, MCHR1, MCHR2, MLNR, MRGPRD, MRGPRE, MRGPRF, MRGPRX1, MRGPRX2, MRGPRX4, MTNR1A, NMBR, NMU1R, NPBWR1, NPBWR2, NPFFR1, NPSR1B, NPY1R, NPY2R, NTSR1, OPN5, OPRD1, OPRK1, OPRL1, OPRM1, OXER1, OXGR1, OXTR, P2RY1, P2RY11, P2RY12, P2RY2, P2RY4, P2RY6, P2RY8, PPYR1, PRLHR, PROKR1, PROKR2, PTAFR, PTGER2, PTGER3, PTGER4, PTGFR, PTGIR, PTHR1, PTHR2, RXFP3, SCTR, SPR4, SSTR1, SSTR2, SSTR3, SSTR5, TAAR5, TACR1, TACR2, TACR3, TBXA2R, TRHR, TSHR(L), UTR2, VIPR1, and VIPR2.

14. The method of claim 13, wherein the GPCR is selected from the group consisting of GPR119, SPR4, G2A, PTGIR, and PTGER4.

15. The method of claim 8, wherein the GPCR activity is reduced.

16. The method of claim 8, wherein the GPCR activity is increased.

17. A method for treating a disease or disorder in a subject, the method comprising administering to a subject a therapeutically effective amount of a composition comprising at least one selected from the group consisting of a genetically engineered cell, an hm-NAS gene, and a N-acyl amide, wherein the cell expresses a human microbial N-acyl synthase (hm-NAS) gene.

18. The method of claim 17, wherein the hm-NAS gene is selected from a hm-NAS gene of table 1 or table 2.

19. The method of claim 17, wherein the a N-acyl amide is represented by Formula (1): 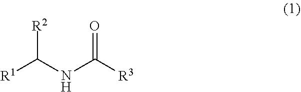
wherein R¹ is selected from the group consisting of carboxylate and CH₂OH;
R² is selected from the group consisting of H, (C₃-C₄)alkyl-NH₃⁺, (C₃-C₄)alkyl-NH₂, C₂ alkyl-C(═O)NH₂, CH₂OH, and methyl; and
R³ is selected from the group consisting of (C₉-C₁₈)alkyl, (C₉-C₁₈)alkenyl, wherein the (C₉-C₁₈)alkyl and (C₉-C₁₈)alkenyl are optionally substituted.

21. The method of claim 17, wherein the disease or disorder is selected from the group consisting of diabetes, obesity, colitis, autoimmune disorder, atherosclerosis, gastrophoresis, cirrhosis, non-alcoholic fatty liver disease, non-alcoholic steatohepatitis, and osteopenia.

22. The method of claim 17, wherein the disease or disorder is associated with abnormal gastric emptying, appetite, or glucose homeostasis.

23. The method of claim 17, wherein the subject is a mammal.

24. The method of claim 17, wherein the subject is a human.

25. A gene therapy vector, comprising a nucleic acid expression cassette, wherein the nucleic acid expression cassette comprises a sequence of a hm-NAS gene or a sequence having at least 90% homology to a hm-NAS gene.

26. The gene therapy vector of claim 23, wherein the hm-NAS gene is selected from a hm-NAS gene of table 1 or table 2.

27. The gene therapy vector of claim 23, wherein the gene therapy vector is selected from the group consisting of a lentiviral vector, a retroviral vector and an adenoviral vector.

28. A composition comprising an N-acyl amide, wherein the N-acyl amide is represented by Formula (1): 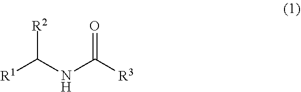
wherein R¹ is selected from the group consisting of carboxylate and CH₂OH;
R² is selected from the group consisting of H, (C₃-C₄)alkyl-NH₃⁺, (C₃-C₄)alkyl-NH₂, C₂ alkyl-C(═O)NH₂, CH₂OH, and methyl; and
R³ is selected from the group consisting of (C₉-C₁₈)alkyl, (C₉-C₁₈)alkenyl, wherein the (C₉-C₁₈)alkyl and (C₉-C₁₈)alkenyl are optionally substituted.

29. The composition of claim 26, wherein Formula (1) is represented by one of Formulae (2)-(6): 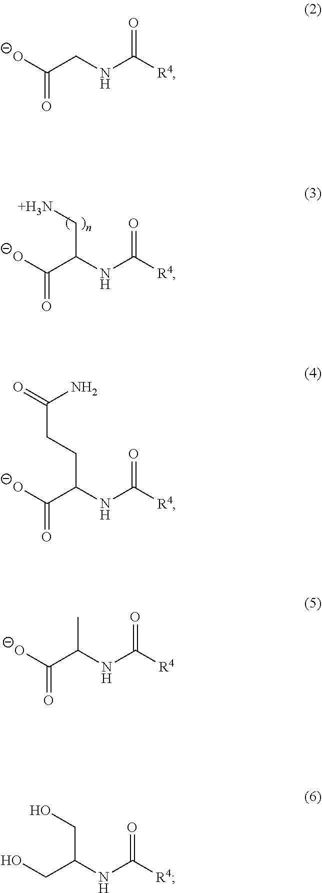
wherein R⁴ is selected from the group consisting of (C₉-C₁₈)alkyl, (C₉-C₁₈)alkenyl, wherein the (C₉-C₁₈)alkyl and (C₉-C₁₈)alkenyl are optionally substituted; and
n is 3 or 4.

30. The composition of claim 27, wherein Formulae (2)-(6) are represented by Formulae (7)-(11): 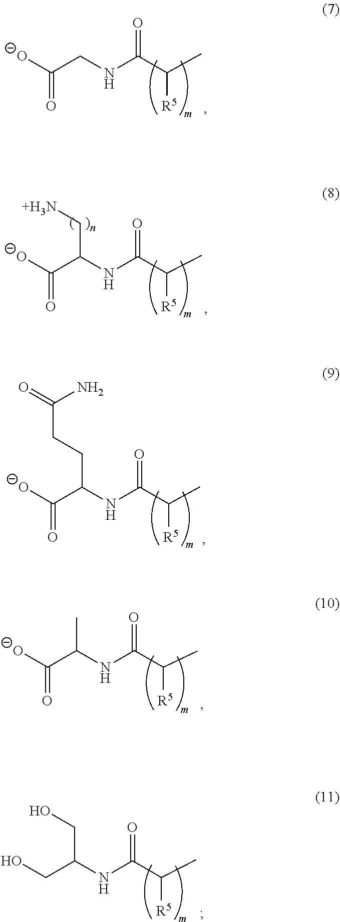
wherein each occurrence of R⁵ is independently selected from the group consisting of H and —OH;
and m is an integer from 8 to 17.

31. The composition of claim 27, wherein Formulae (2)-(6) are represented by Formulae (12)-(16): 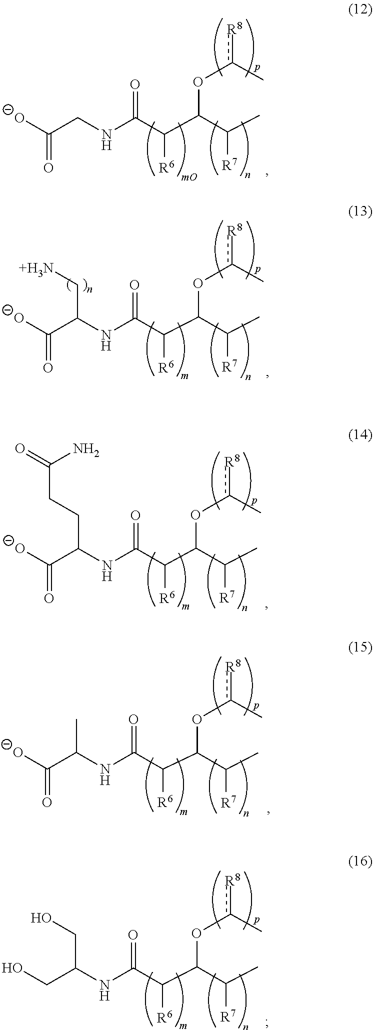
wherein each occurrence of R⁶, R⁷, and R⁸ is independently selected from the group consisting of H, —OH, and (═O);
m is an integer from 1 to 5;
n is an integer from 2 to 15;
p is an integer from 8 to 18; and
q is an integer from 3 to 4.

32. The composition of claim 26, wherein the N-acyl amide is selected from the group consisting of

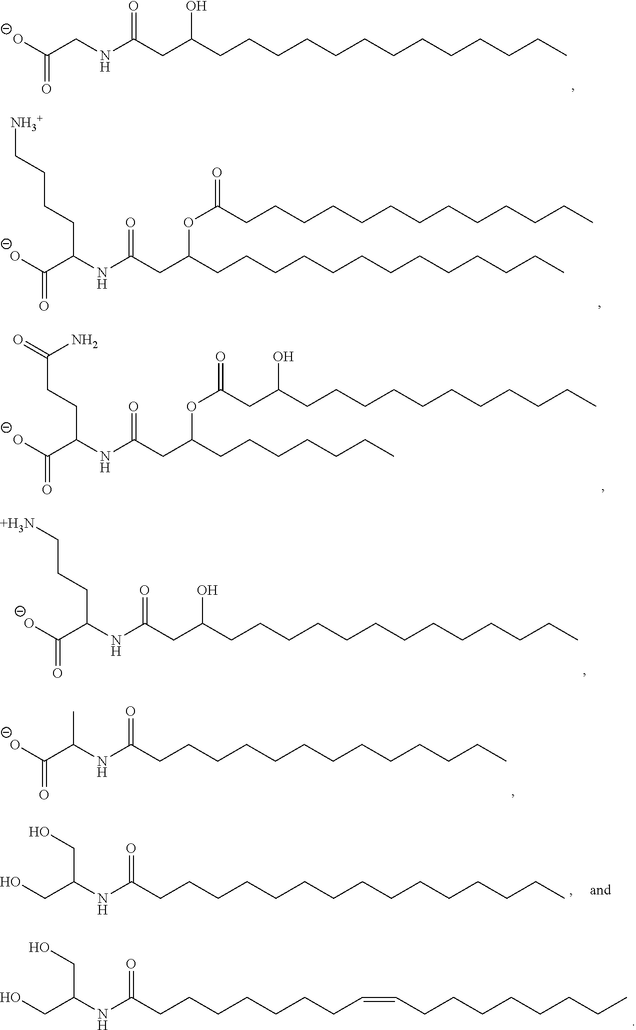

33. The composition of claim 26, wherein the composition further comprises a pharmaceutically acceptable carrier.

34. The composition of claim 31, wherein the composition is formulated as a probiotic.
